(19) United States
(12) Patent Application Publication
(10) Pub. No.: US 2022/0257556 A1
(43) Pub. Date: Aug. 18, 2022

(54) DOSING REGIMENS FOR ORAL COMPLEMENT FACTOR D INHIBITORS

(71) Applicant: BioCryst Pharmaceuticals, Inc., Durham, NC (US)

(72) Inventors: Yarlagadda S. Babu, Birmingham, AL (US); William P. Sheridan, Durham, NC (US)

(21) Appl. No.: 17/630,692

(22) PCT Filed: Jul. 29, 2020
(86) PCT No.: PCT/US20/44037

§ 371 Date: Jan. 27, 2022

Related U.S. Application Data
(60) Provisional application No. 63/020,239, filed on May 5, 2020.
(60) Provisional application No. 62/926,175, filed on Oct. 25, 2019.
(60) Provisional application No. 62/881,225, filed on Jul. 31, 2019.

(51) Int. Cl. A61K 31/343; A61K 9/00; A61K 31/443; A61K 31/4436; A61K 31/4725; A61K 31/5377; A61K 31/4178; A61K 31/423; A61K 31/53; A61K 31/416; A61K 31/381; A61K 31/4525; A61K 31/4355; A61K 31/519; A61K 31/4025; A61P 7/06; A61P 37/06

(52) U.S. Cl. CPC A61K 31/343; A61P 37/06; A61K 31/443; A61K 31/4436; A61K 31/4725; A61K 31/5377; A61K 31/4178; A61K 31/423; A61K 31/53; A61K 31/416; A61K 31/381; A61K 31/4525; A61K 31/4355; A61K 31/519; A61K 31/4025; A61P 7/06; A61K 9/0053

(57) ABSTRACT

Disclosed are compounds and pharmaceutically acceptable salts and prodrugs thereof, which are inhibitors of the complement system. Also provided are oral dosage forms comprising such a compound, salt, or prodrug. Also disclosed are methods of using the compounds, salts, and prodrugs, and oral dosage forms thereof, in the treatment or prevention of a disease or condition characterized by aberrant complement system activity (e.g., paroxysmal nocturnal hemoglobinuria).

### RELATED APPLICATIONS

This application claims the benefit of priority to U.S. Provisional Patent Application No. 62/881,225, filed Jul. 31, 2019; U.S. Provisional Patent Application No. 62/926,175, filed Oct. 25, 2019; and U.S. Provisional Patent Application No. 63/020,239, filed May 5, 2020.

### BACKGROUND

The complement system is a branch of an organism's immune system that enhances the ability of antibodies and phagocytic cells to destroy and remove foreign particles (e.g., pathogens) from the organism. The complement system comprises a set of plasma proteins that act together to attack extracellular forms of pathogens and induce a series of inflammatory responses to help fight infection. Complement activation can occur through several pathways. For example, complement activation can occur spontaneously in response to certain pathogens or by antibody binding to a pathogen. When complement proteins are activated a cascade is triggered by which one complement protein induces the activation of the next protein in the sequence. The activation of a small number of complement proteins at the start of the pathway is hugely amplified by each successive enzymatic reaction, resulting in the rapid generation of a disproportionately large complement response. (Marrides, S. Pharmacological Reviews 1998, Vol. 50, pages 59-88). In healthy organisms there are regulatory mechanisms to prevent uncontrolled complement activation.

When activated, complement proteins can bind to a pathogen, opsonizing them for engulfment by phagocytes bearing receptors for complement. Then, small fragments of some complement proteins act as chemoattractants to recruit more phagocytes to the site of complement activation, and also to activate these phagocytes. Next, the complement proteins create holes or pores in the invading organisms, leading to their destruction. While complement plays an important role in protecting the body from foreign organisms, it can also destroy healthy cells and tissue. The inappropriate activation of complement is implicated in a long list of disease pathologies (Morgan, B. Eur J Clin Invest 1994, Vol. 24, pages 219-228) affecting the immune, renal, cardiovascular, and neurological systems. Accordingly, there exists a need to develop further complement inhibitors, which have therapeutic potential in the treatment of numerous disorders.

### SUMMARY

In certain aspects, the invention provides an oral dosage form comprising a compound selected from:

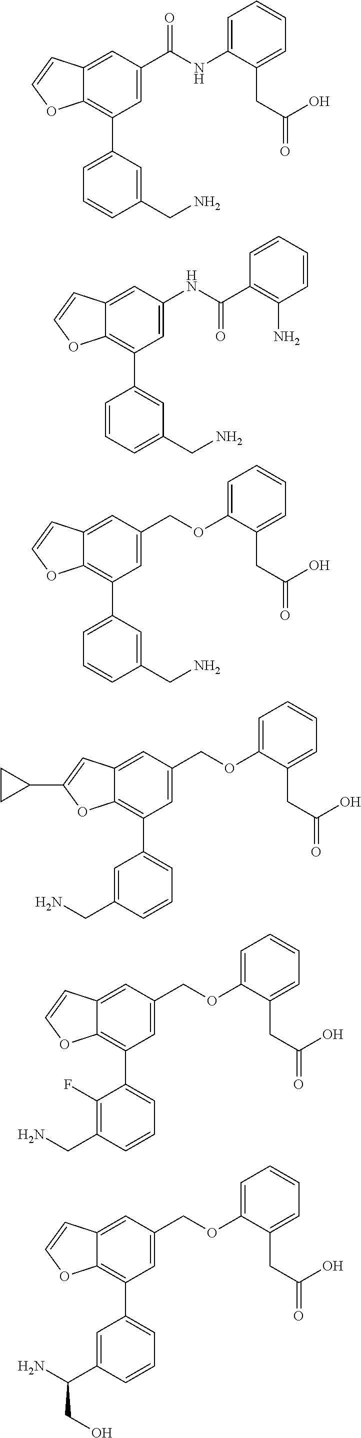

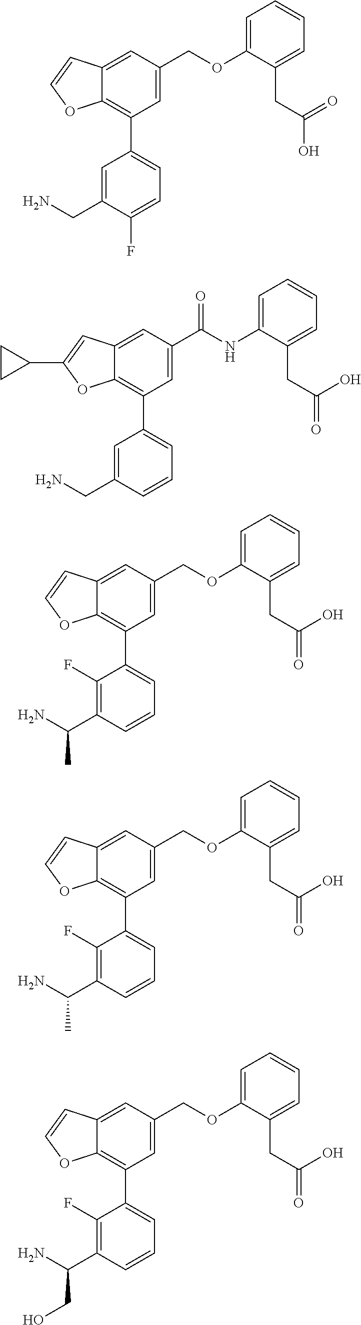 [SMILES: NCc1cc(-c2cc(COc3ccccc3CC(=O)O)cc3ccoc23)ccc1F ; NCc1cccc(-c2cc(C(=O)Nc3ccccc3CC(=O)O)cc3cc(C4CC4)oc23)c1 ; C[C@@H](N)c1cccc(-c2cc(COc3ccccc3CC(=O)O)cc3ccoc23)c1F ; C[C@H](N)c1cccc(-c2cc(COc3ccccc3CC(=O)O)cc3ccoc23)c1F ; N[C@H](CO)c1cccc(-c2cc(COc3ccccc3CC(=O)O)cc3ccoc23)c1F]

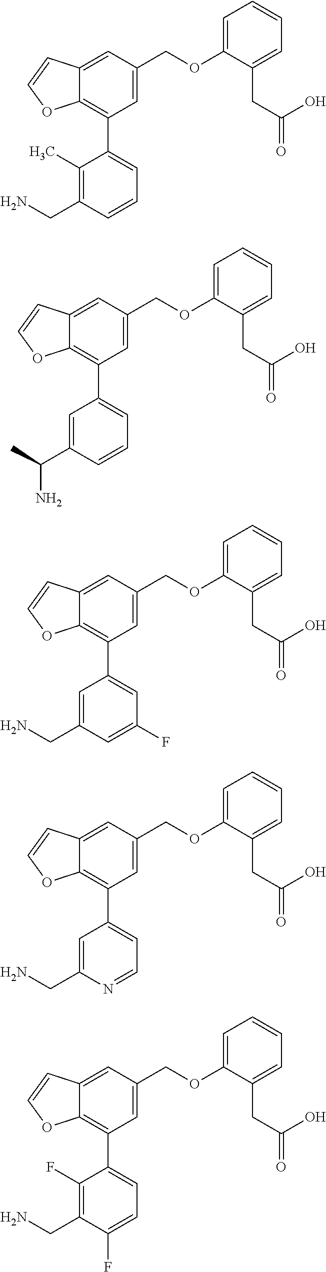 [SMILES: Cc1c(CN)cccc1-c1cc(COc2ccccc2CC(=O)O)cc2ccoc12 ; C[C@H](N)c1cccc(-c2cc(COc3ccccc3CC(=O)O)cc3ccoc23)c1 ; NCc1cc(F)cc(-c2cc(COc3ccccc3CC(=O)O)cc3ccoc23)c1 ; NCc1cc(-c2cc(COc3ccccc3CC(=O)O)cc3ccoc23)ccn1 ; NCc1c(F)ccc(-c2cc(COc3ccccc3CC(=O)O)cc3ccoc23)c1F]

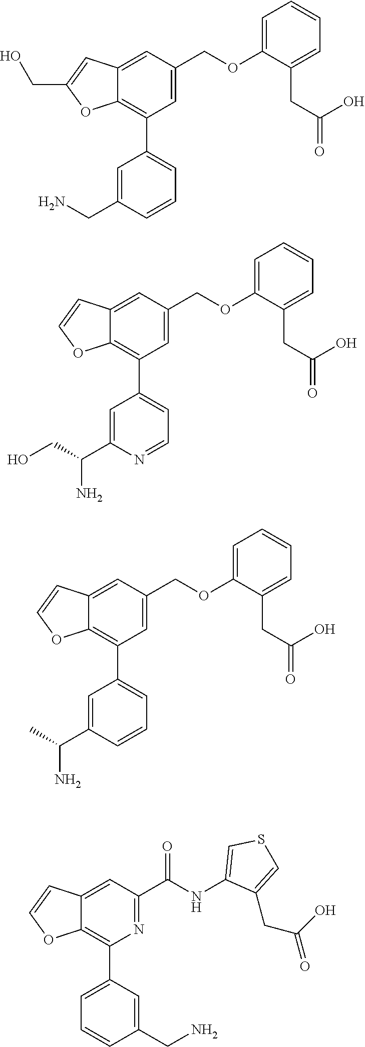 [SMILES: NCc1cccc(-c2cc(COc3ccccc3CC(=O)O)cc3cc(CO)oc23)c1 ; N[C@H](CO)c1cc(-c2cc(COc3ccccc3CC(=O)O)cc3ccoc23)ccn1 ; C[C@@H](N)c1cccc(-c2cc(COc3ccccc3CC(=O)O)cc3ccoc23)c1 ; NCc1cccc(-c2nc(C(=O)Nc3cscc3CC(=O)O)cc3ccoc23)c1]

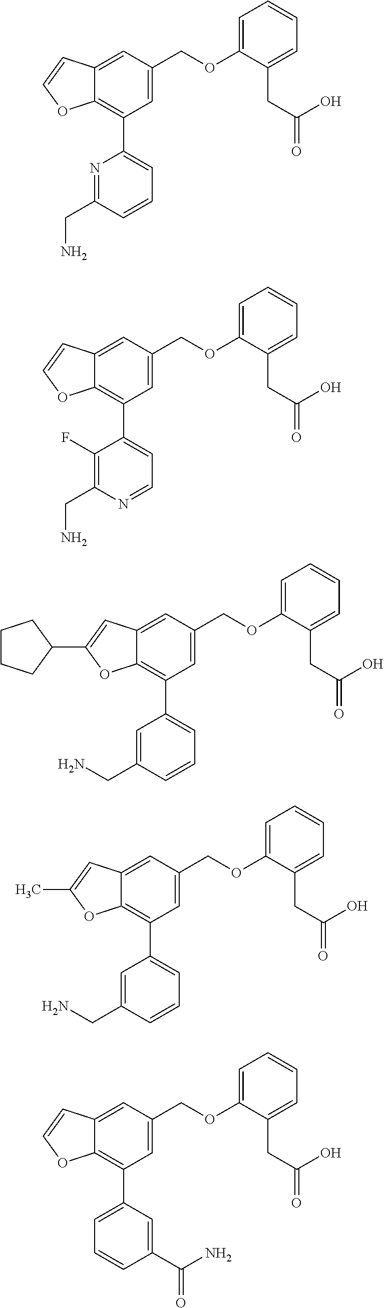 [SMILES: NCc1cccc(-c2cc(COc3ccccc3CC(=O)O)cc3ccoc23)n1 ; NCc1nccc(-c2cc(COc3ccccc3CC(=O)O)cc3ccoc23)c1F ; NCc1cccc(-c2cc(COc3ccccc3CC(=O)O)cc3cc(C4CCCC4)oc23)c1 ; Cc1cc2cc(COc3ccccc3CC(=O)O)cc(-c3cccc(CN)c3)c2o1 ; NC(=O)c1cccc(-c2cc(COc3ccccc3CC(=O)O)cc3ccoc23)c1]

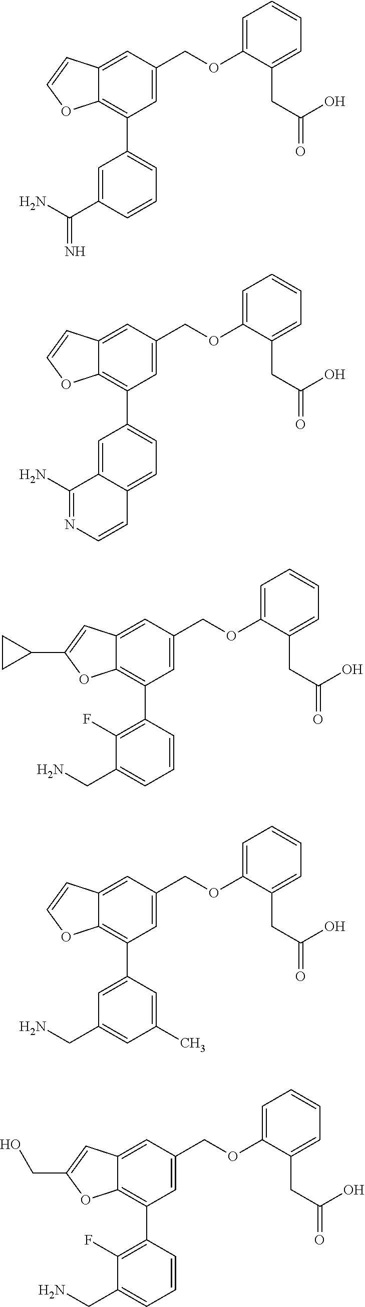 [SMILES: N=C(N)c1cccc(-c2cc(COc3ccccc3CC(=O)O)cc3ccoc23)c1 ; Nc1nccc2ccc(-c3cc(COc4ccccc4CC(=O)O)cc4ccoc34)cc12 ; NCc1cccc(-c2cc(COc3ccccc3CC(=O)O)cc3cc(C4CC4)oc23)c1F ; Cc1cc(CN)cc(-c2cc(COc3ccccc3CC(=O)O)cc3ccoc23)c1 ; NCc1cccc(-c2cc(COc3ccccc3CC(=O)O)cc3cc(CO)oc23)c1F]

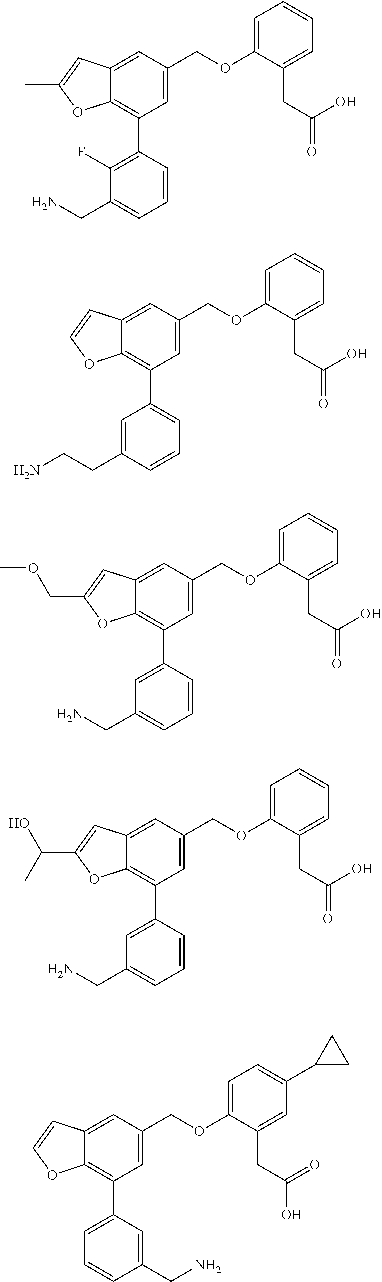 [SMILES: Cc1cc2cc(COc3ccccc3CC(=O)O)cc(-c3cccc(CN)c3F)c2o1 ; NCCc1cccc(-c2cc(COc3ccccc3CC(=O)O)cc3ccoc23)c1 ; COCc1cc2cc(COc3ccccc3CC(=O)O)cc(-c3cccc(CN)c3)c2o1 ; CC(O)c1cc2cc(COc3ccccc3CC(=O)O)cc(-c3cccc(CN)c3)c2o1 ; NCc1cccc(-c2cc(COc3ccc(C4CC4)cc3CC(=O)O)cc3ccoc23)c1]

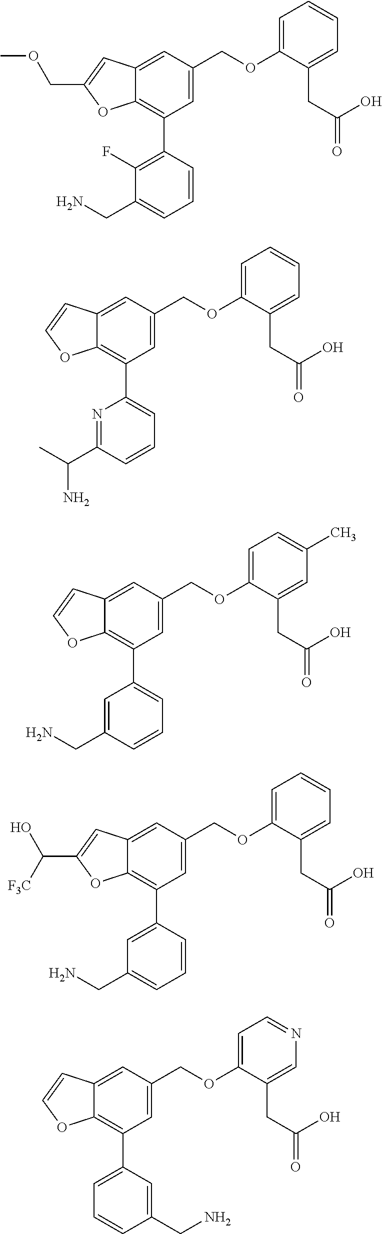 [SMILES: COCc1cc2cc(COc3ccccc3CC(=O)O)cc(-c3cccc(CN)c3F)c2o1 ; CC(N)c1cccc(-c2cc(COc3ccccc3CC(=O)O)cc3ccoc23)n1 ; Cc1ccc(OCc2cc(-c3cccc(CN)c3)c3occc3c2)c(CC(=O)O)c1 ; NCc1cccc(-c2cc(COc3ccccc3CC(=O)O)cc3cc(C(O)C(F)(F)F)oc23)c1 ; NCc1cccc(-c2cc(COc3ccncc3CC(=O)O)cc3ccoc23)c1]

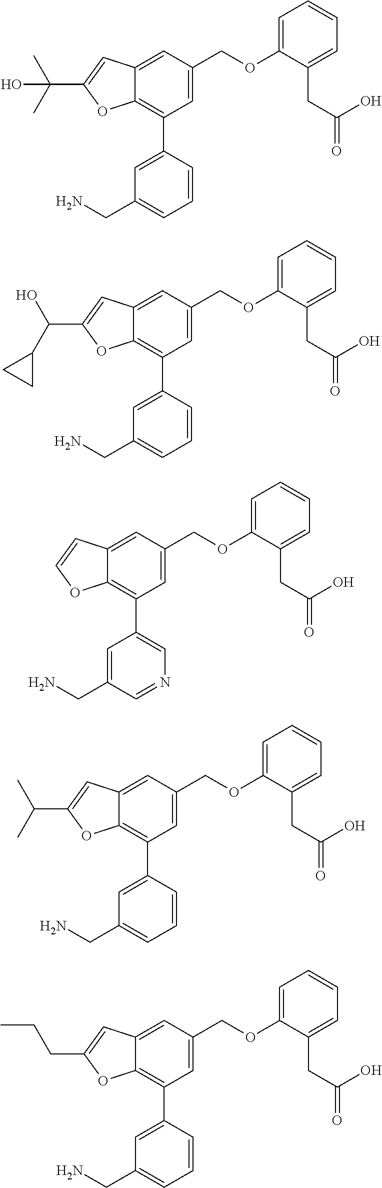 [SMILES: CC(C)(O)c1cc2cc(COc3ccccc3CC(=O)O)cc(-c3cccc(CN)c3)c2o1 ; NCc1cccc(-c2cc(COc3ccccc3CC(=O)O)cc3cc(C(O)C4CC4)oc23)c1 ; NCc1cncc(-c2cc(COc3ccccc3CC(=O)O)cc3ccoc23)c1 ; CC(C)c1cc2cc(COc3ccccc3CC(=O)O)cc(-c3cccc(CN)c3)c2o1 ; CCCc1cc2cc(COc3ccccc3CC(=O)O)cc(-c3cccc(CN)c3)c2o1]

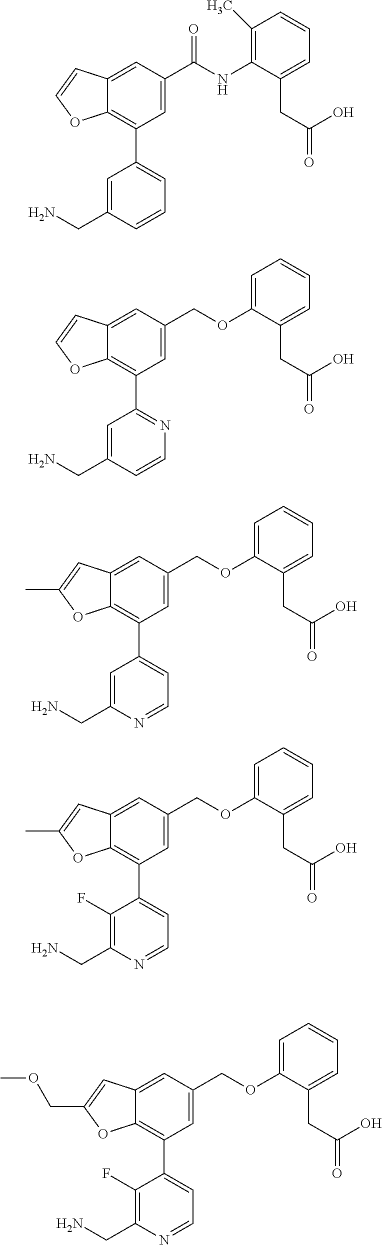 [SMILES: Cc1cccc(CC(=O)O)c1NC(=O)c1cc(-c2cccc(CN)c2)c2occc2c1 ; NCc1ccnc(-c2cc(COc3ccccc3CC(=O)O)cc3ccoc23)c1 ; Cc1cc2cc(COc3ccccc3CC(=O)O)cc(-c3ccnc(CN)c3)c2o1 ; Cc1cc2cc(COc3ccccc3CC(=O)O)cc(-c3ccnc(CN)c3F)c2o1 ; COCc1cc2cc(COc3ccccc3CC(=O)O)cc(-c3ccnc(CN)c3F)c2o1]

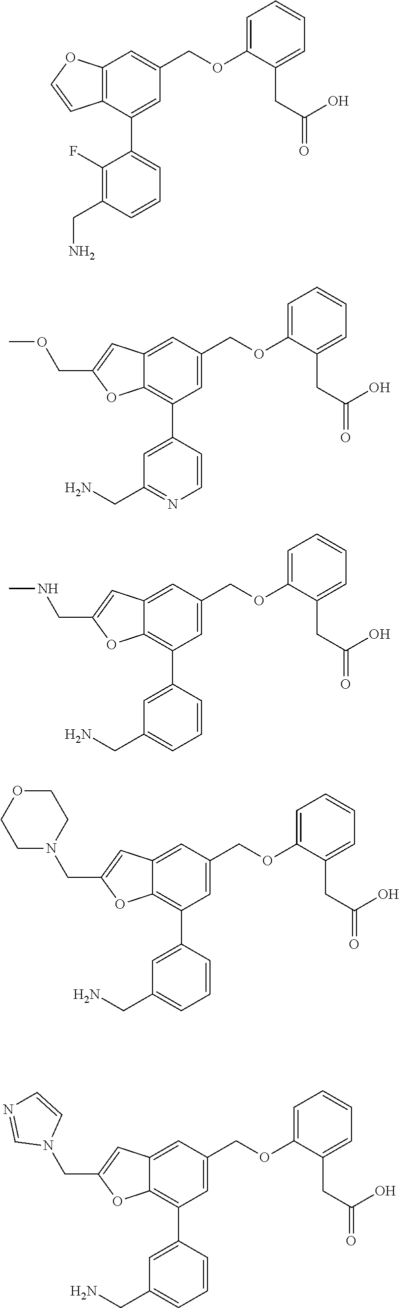 [SMILES: NCc1cccc(-c2cc(COc3ccccc3CC(=O)O)cc3occc23)c1F ; COCc1cc2cc(COc3ccccc3CC(=O)O)cc(-c3ccnc(CN)c3)c2o1 ; CNCc1cc2cc(COc3ccccc3CC(=O)O)cc(-c3cccc(CN)c3)c2o1 ; NCc1cccc(-c2cc(COc3ccccc3CC(=O)O)cc3cc(CN4CCOCC4)oc23)c1 ; NCc1cccc(-c2cc(COc3ccccc3CC(=O)O)cc3cc(Cn4ccnc4)oc23)c1]

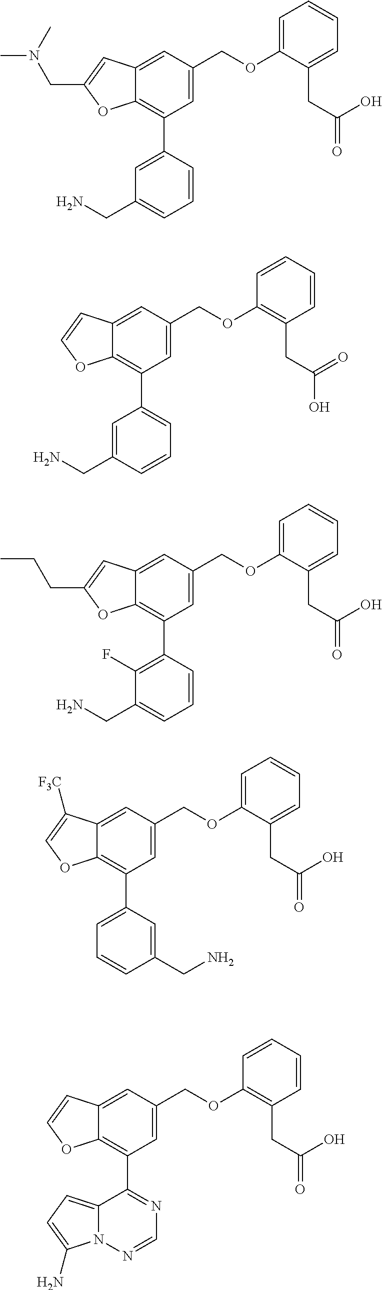 [SMILES: CN(C)Cc1cc2cc(COc3ccccc3CC(=O)O)cc(-c3cccc(CN)c3)c2o1 ; NCc1cccc(-c2cc(COc3ccccc3CC(=O)O)cc3ccoc23)c1 ; CCCc1cc2cc(COc3ccccc3CC(=O)O)cc(-c3cccc(CN)c3F)c2o1 ; NCc1cccc(-c2cc(COc3ccccc3CC(=O)O)cc3c(C(F)(F)F)coc23)c1 ; Nc1ccc2c(-c3cc(COc4ccccc4CC(=O)O)cc4ccoc34)ncnn12]

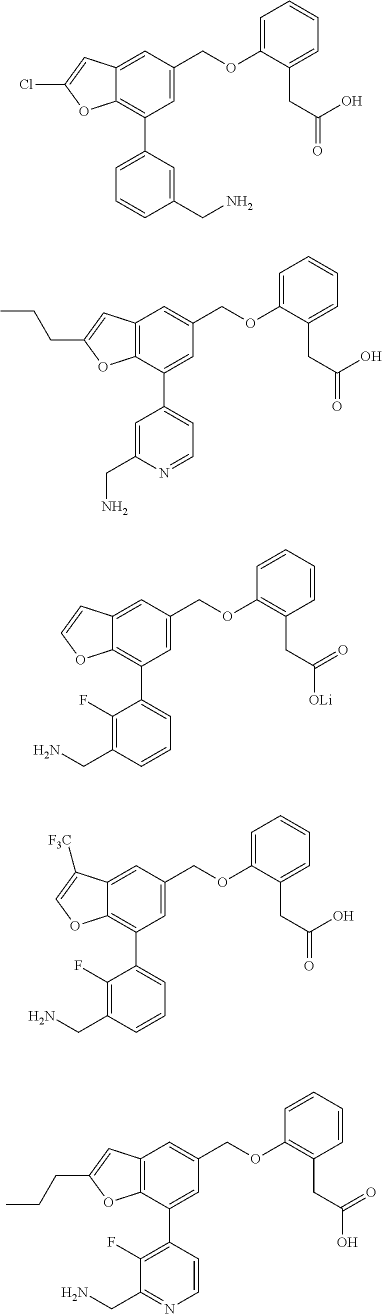

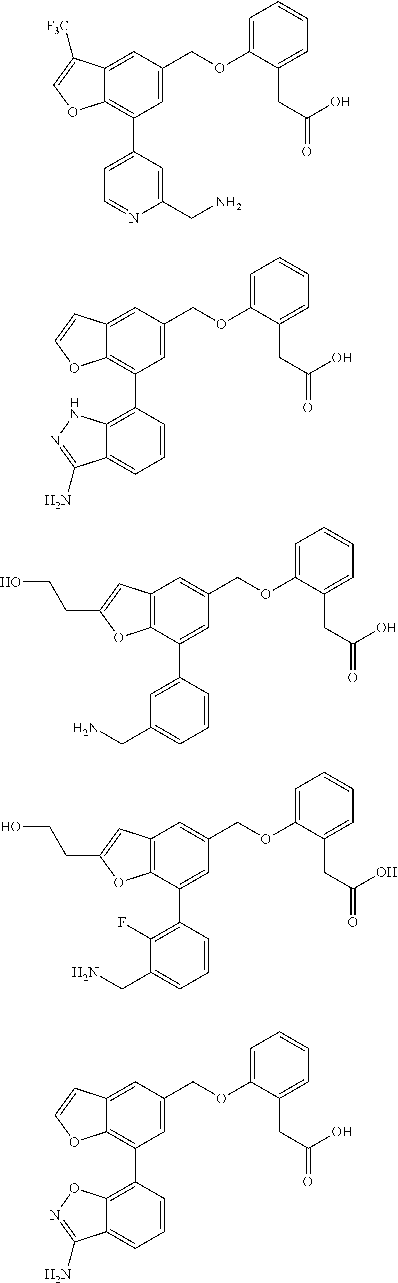 [SMILES: NCc1cc(-c2cc(COc3ccccc3CC(=O)O)cc3c(C(F)(F)F)coc23)ccn1 ; Nc1n[nH]c2c(-c3cc(COc4ccccc4CC(=O)O)cc4ccoc34)cccc12 ; NCc1cccc(-c2cc(COc3ccccc3CC(=O)O)cc3cc(CCO)oc23)c1 ; NCc1cccc(-c2cc(COc3ccccc3CC(=O)O)cc3cc(CCO)oc23)c1F ; Nc1noc2c(-c3cc(COc4ccccc4CC(=O)O)cc4ccoc34)cccc12]

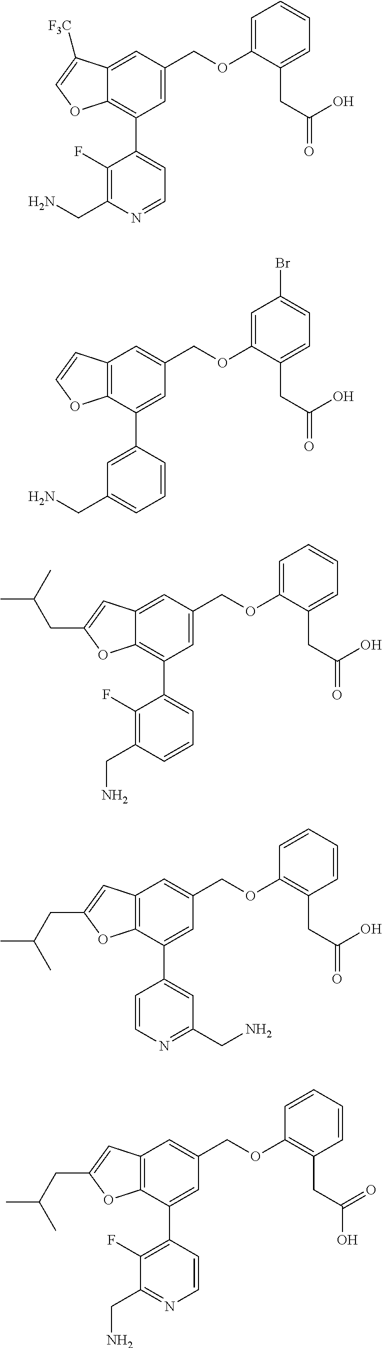 [SMILES: NCc1nccc(-c2cc(COc3ccccc3CC(=O)O)cc3c(C(F)(F)F)coc23)c1F ; NCc1cccc(-c2cc(COc3cc(Br)ccc3CC(=O)O)cc3ccoc23)c1 ; CC(C)Cc1cc2cc(COc3ccccc3CC(=O)O)cc(-c3cccc(CN)c3F)c2o1 ; CC(C)Cc1cc2cc(COc3ccccc3CC(=O)O)cc(-c3ccnc(CN)c3)c2o1 ; CC(C)Cc1cc2cc(COc3ccccc3CC(=O)O)cc(-c3ccnc(CN)c3F)c2o1]

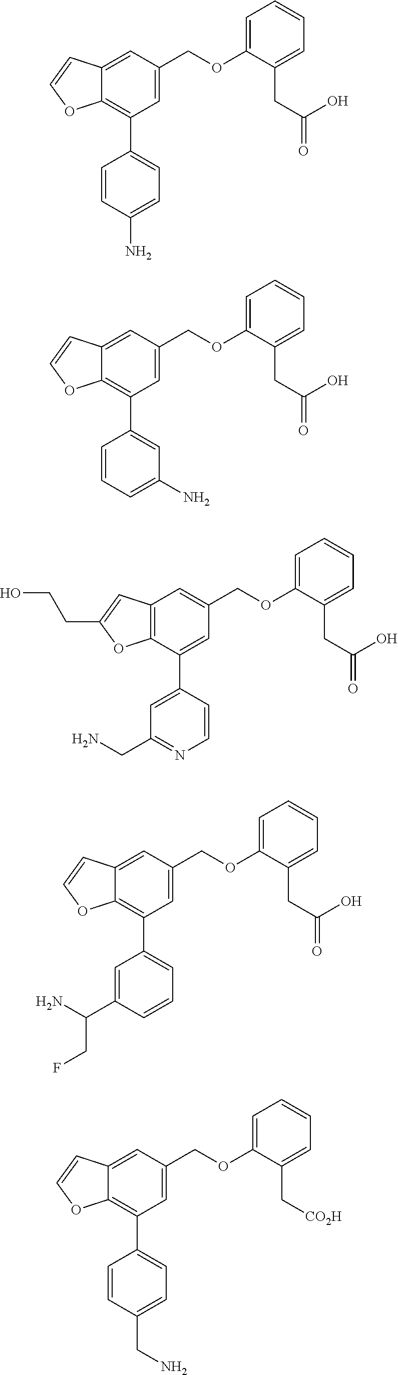 [SMILES: Nc1ccc(-c2cc(COc3ccccc3CC(=O)O)cc3ccoc23)cc1 ; Nc1cccc(-c2cc(COc3ccccc3CC(=O)O)cc3ccoc23)c1 ; NCc1cc(-c2cc(COc3ccccc3CC(=O)O)cc3cc(CCO)oc23)ccn1 ; NC(CF)c1cccc(-c2cc(COc3ccccc3CC(=O)O)cc3ccoc23)c1 ; NCc1ccc(-c2cc(COc3ccccc3CC(=O)O)cc3ccoc23)cc1]

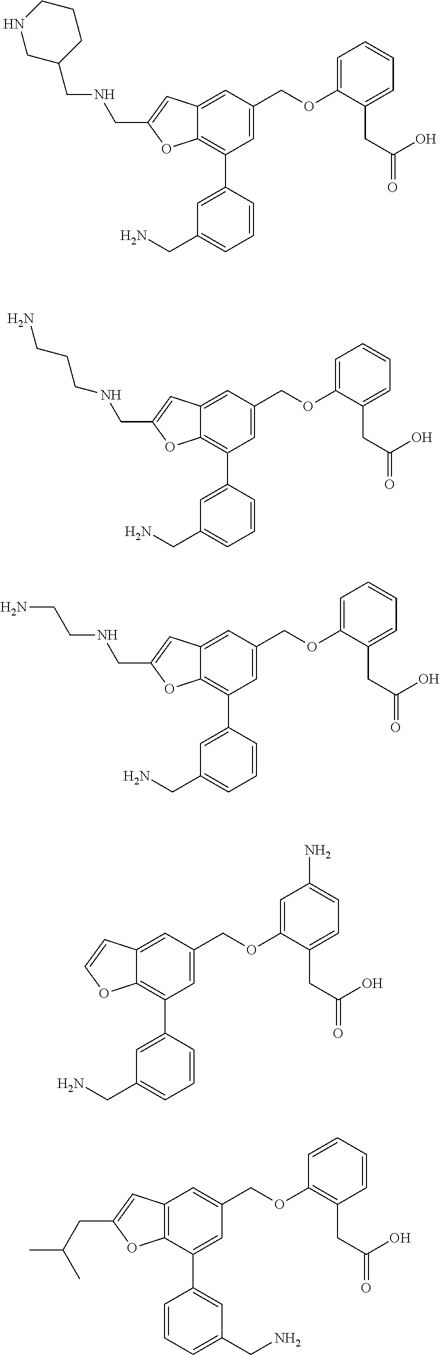 [SMILES: NCc1cccc(-c2cc(COc3ccccc3CC(=O)O)cc3cc(CNCC4CCCNC4)oc23)c1 ; NCCCNCc1cc2cc(COc3ccccc3CC(=O)O)cc(-c3cccc(CN)c3)c2o1 ; NCCNCc1cc2cc(COc3ccccc3CC(=O)O)cc(-c3cccc(CN)c3)c2o1 ; NCc1cccc(-c2cc(COc3cc(N)ccc3CC(=O)O)cc3ccoc23)c1 ; CC(C)Cc1cc2cc(COc3ccccc3CC(=O)O)cc(-c3cccc(CN)c3)c2o1]

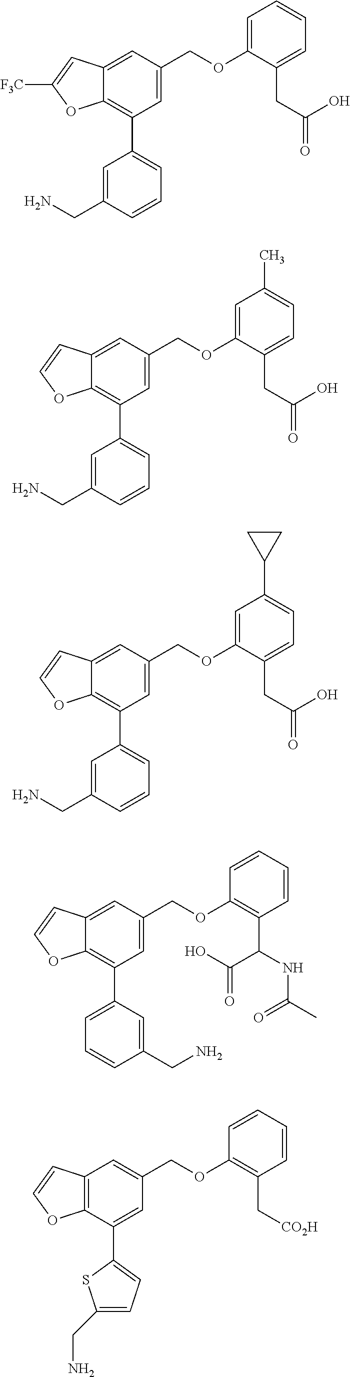 [SMILES: NCc1cccc(-c2cc(COc3ccccc3CC(=O)O)cc3cc(C(F)(F)F)oc23)c1 ; Cc1ccc(CC(=O)O)c(OCc2cc(-c3cccc(CN)c3)c3occc3c2)c1 ; NCc1cccc(-c2cc(COc3cc(C4CC4)ccc3CC(=O)O)cc3ccoc23)c1 ; CC(=O)NC(C(=O)O)c1ccccc1OCc1cc(-c2cccc(CN)c2)c2occc2c1 ; NCc1ccc(-c2cc(COc3ccccc3CC(=O)O)cc3ccoc23)s1]

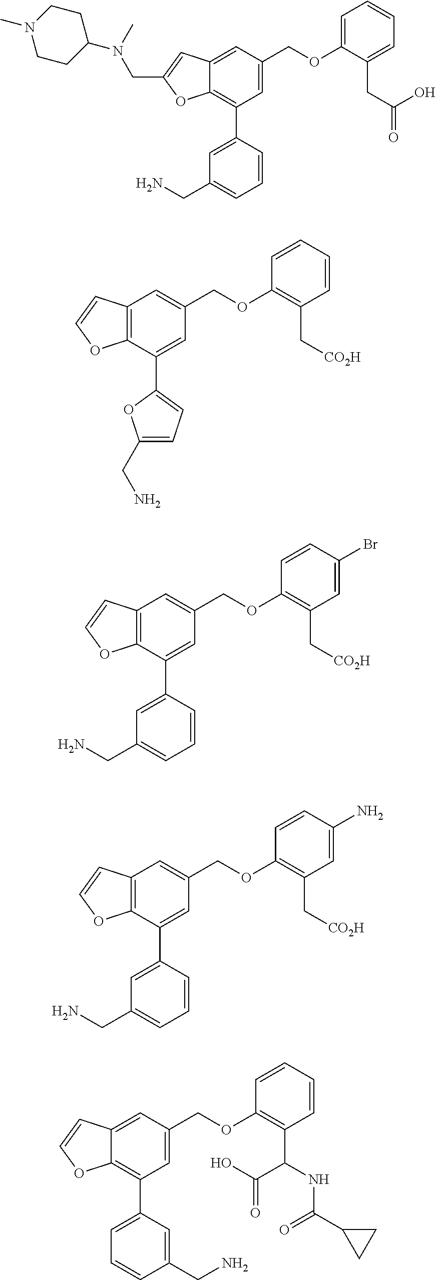 [SMILES: CN1CCC(N(C)Cc2cc3cc(COc4ccccc4CC(=O)O)cc(-c4cccc(CN)c4)c3o2)CC1 ; NCc1ccc(-c2cc(COc3ccccc3CC(=O)O)cc3ccoc23)o1 ; NCc1cccc(-c2cc(COc3ccc(Br)cc3CC(=O)O)cc3ccoc23)c1 ; NCc1cccc(-c2cc(COc3ccc(N)cc3CC(=O)O)cc3ccoc23)c1 ; NCc1cccc(-c2cc(COc3ccccc3C(NC(=O)C3CC3)C(=O)O)cc3ccoc23)c1]

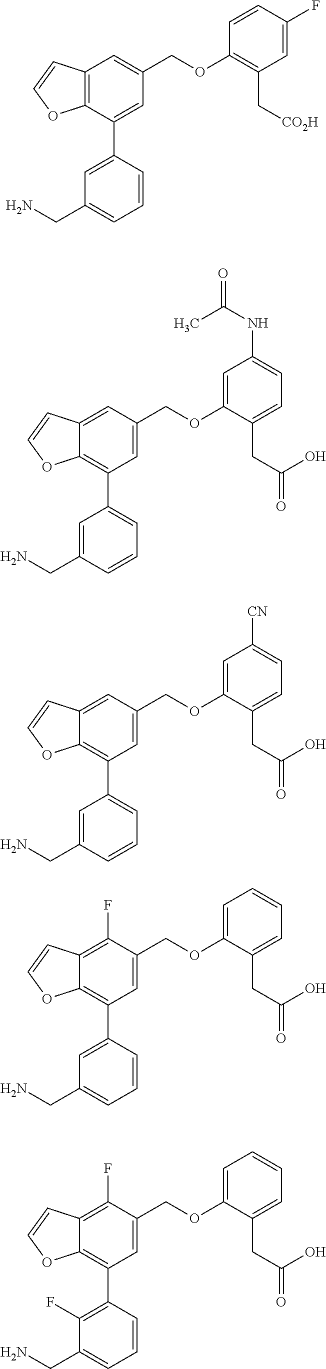

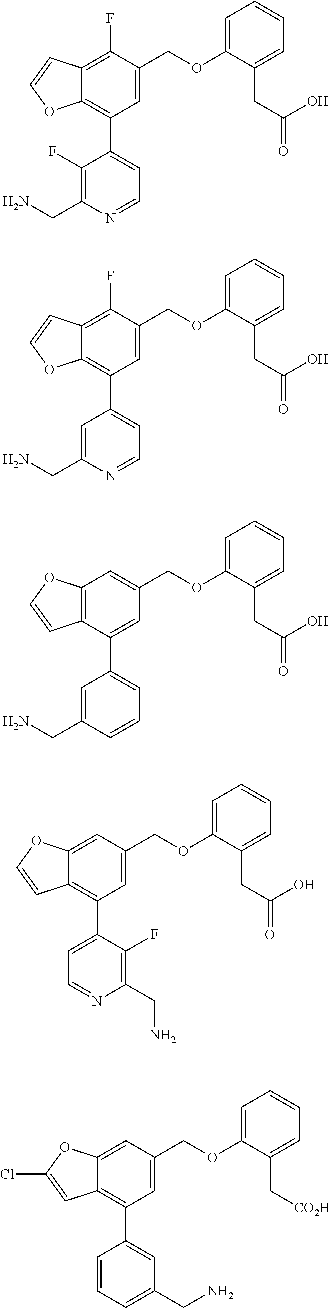 [SMILES: NCc1nccc(-c2cc(COc3ccccc3CC(=O)O)c(F)c3ccoc23)c1F ; NCc1cc(-c2cc(COc3ccccc3CC(=O)O)c(F)c3ccoc23)ccn1 ; NCc1cccc(-c2cc(COc3ccccc3CC(=O)O)cc3occc23)c1 ; NCc1nccc(-c2cc(COc3ccccc3CC(=O)O)cc3occc23)c1F ; NCc1cccc(-c2cc(COc3ccccc3CC(=O)O)cc3oc(Cl)cc23)c1]

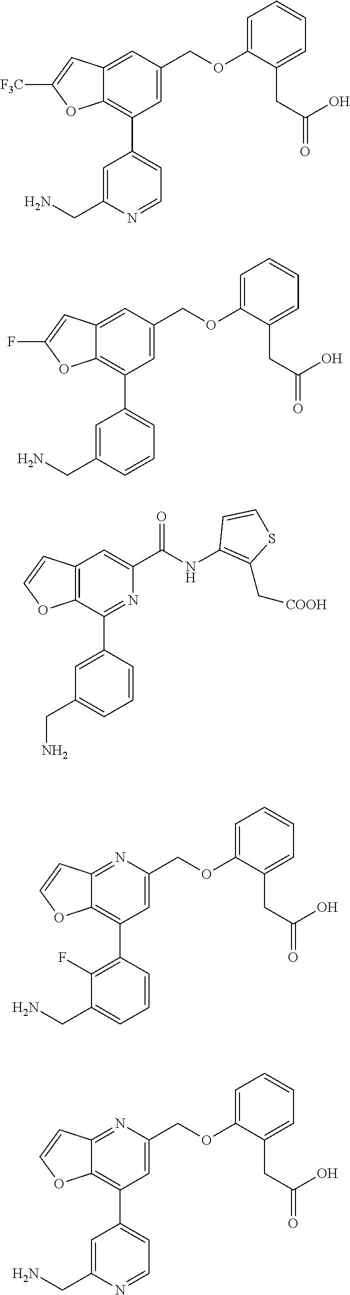 [SMILES: NCc1cc(-c2cc(COc3ccccc3CC(=O)O)cc3cc(C(F)(F)F)oc23)ccn1 ; NCc1cccc(-c2cc(COc3ccccc3CC(=O)O)cc3cc(F)oc23)c1 ; NCc1cccc(-c2nc(C(=O)Nc3ccsc3CC(=O)O)cc3ccoc23)c1 ; NCc1cccc(-c2cc(COc3ccccc3CC(=O)O)nc3ccoc23)c1F ; NCc1cc(-c2cc(COc3ccccc3CC(=O)O)nc3ccoc23)ccn1]

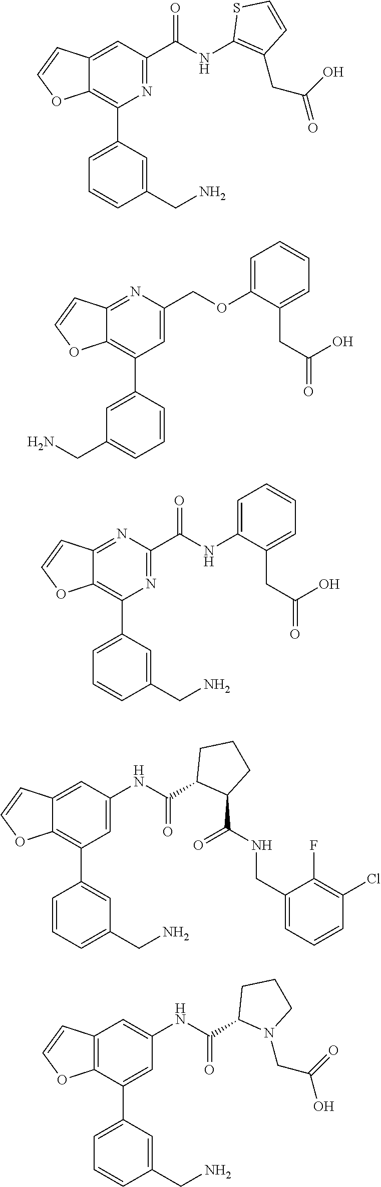 [SMILES: NCc1cccc(-c2nc(C(=O)Nc3sccc3CC(=O)O)cc3ccoc23)c1 ; NCc1cccc(-c2cc(COc3ccccc3CC(=O)O)nc3ccoc23)c1 ; NCc1cccc(-c2nc(C(=O)Nc3ccccc3CC(=O)O)nc3ccoc23)c1 ; NCc1cccc(-c2cc(NC(=O)[C@@H]3CCC[C@H]3C(=O)NCc3cccc(Cl)c3F)cc3ccoc23)c1 ; NCc1cccc(-c2cc(NC(=O)[C@@H]3CCCN3CC(=O)O)cc3ccoc23)c1]

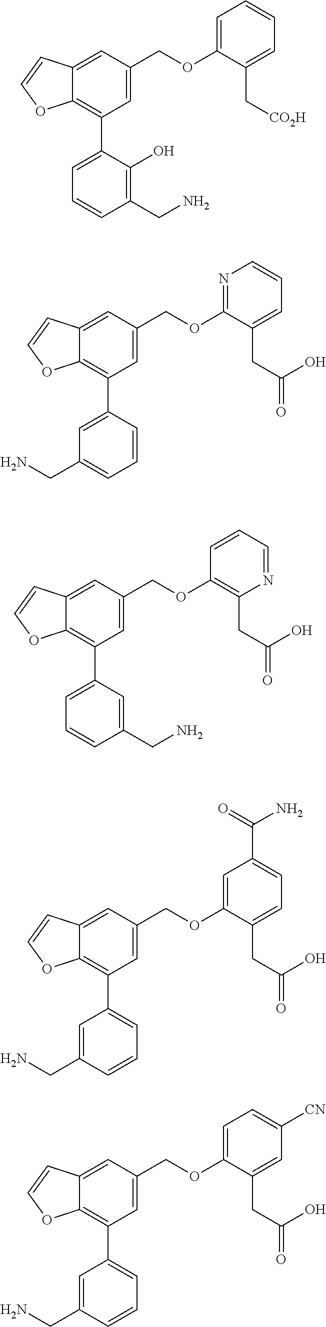 [SMILES: NCc1cccc(-c2cc(COc3ccccc3CC(=O)O)cc3ccoc23)c1O ; NCc1cccc(-c2cc(COc3ncccc3CC(=O)O)cc3ccoc23)c1 ; NCc1cccc(-c2cc(COc3cccnc3CC(=O)O)cc3ccoc23)c1 ; NCc1cccc(-c2cc(COc3cc(C(N)=O)ccc3CC(=O)O)cc3ccoc23)c1 ; N#Cc1ccc(OCc2cc(-c3cccc(CN)c3)c3occc3c2)c(CC(=O)O)c1]

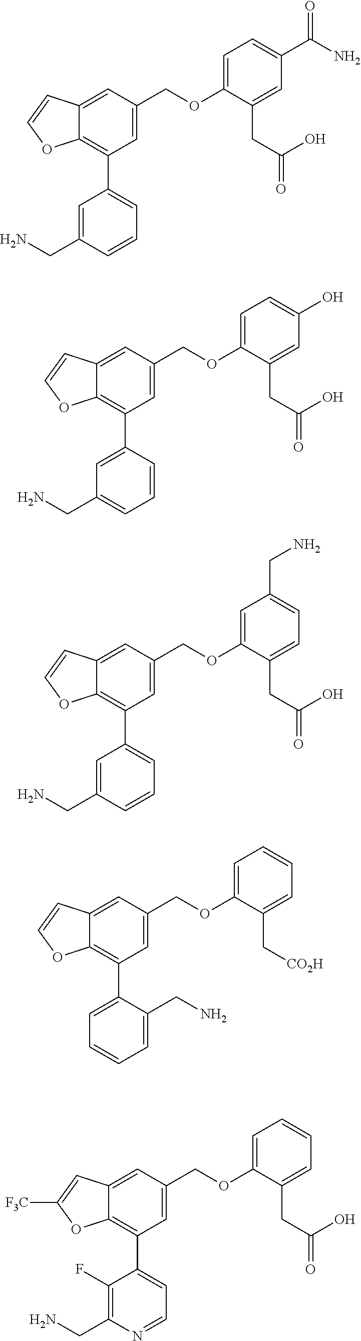 [SMILES: NCc1cccc(-c2cc(COc3ccc(C(N)=O)cc3CC(=O)O)cc3ccoc23)c1 ; NCc1cccc(-c2cc(COc3ccc(O)cc3CC(=O)O)cc3ccoc23)c1 ; NCc1cccc(-c2cc(COc3cc(CN)ccc3CC(=O)O)cc3ccoc23)c1 ; NCc1ccccc1-c1cc(COc2ccccc2CC(=O)O)cc2ccoc12 ; NCc1nccc(-c2cc(COc3ccccc3CC(=O)O)cc3cc(C(F)(F)F)oc23)c1F]

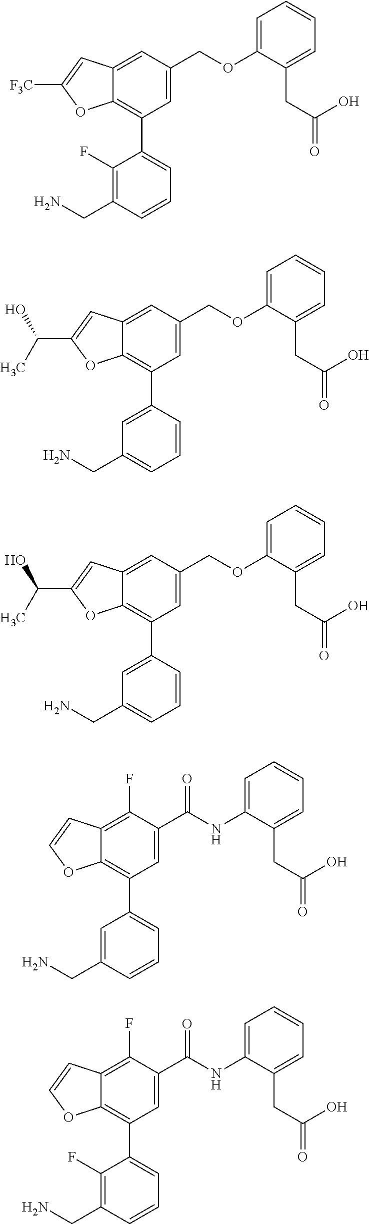

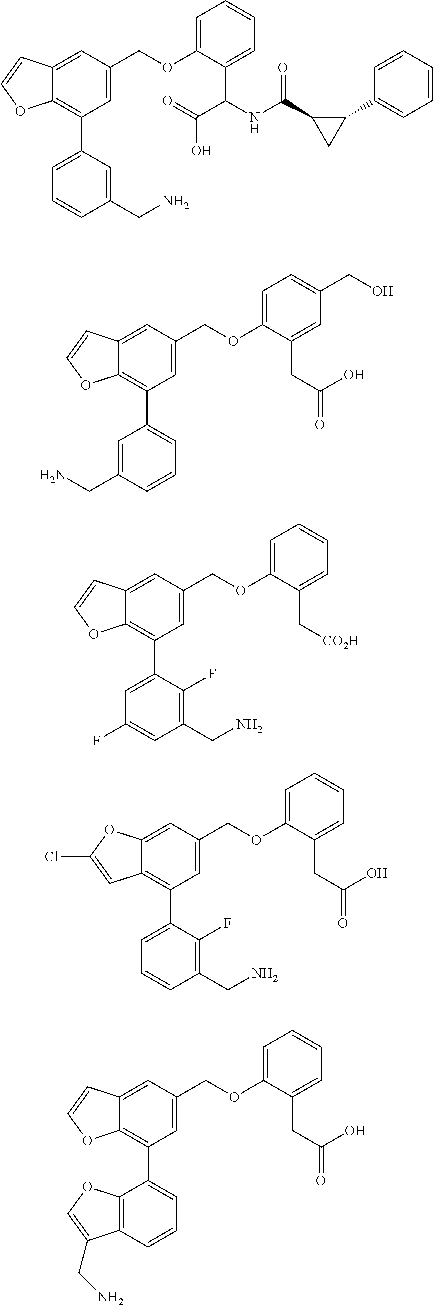 [SMILES: NCc1cccc(-c2cc(COc3ccccc3C(NC(=O)[C@@H]3C[C@H]3c3ccccc3)C(=O)O)cc3ccoc23)c1 ; NCc1cccc(-c2cc(COc3ccc(CO)cc3CC(=O)O)cc3ccoc23)c1 ; NCc1cc(F)cc(-c2cc(COc3ccccc3CC(=O)O)cc3ccoc23)c1F ; NCc1cccc(-c2cc(COc3ccccc3CC(=O)O)cc3oc(Cl)cc23)c1F ; NCc1coc2c(-c3cc(COc4ccccc4CC(=O)O)cc4ccoc34)cccc12]

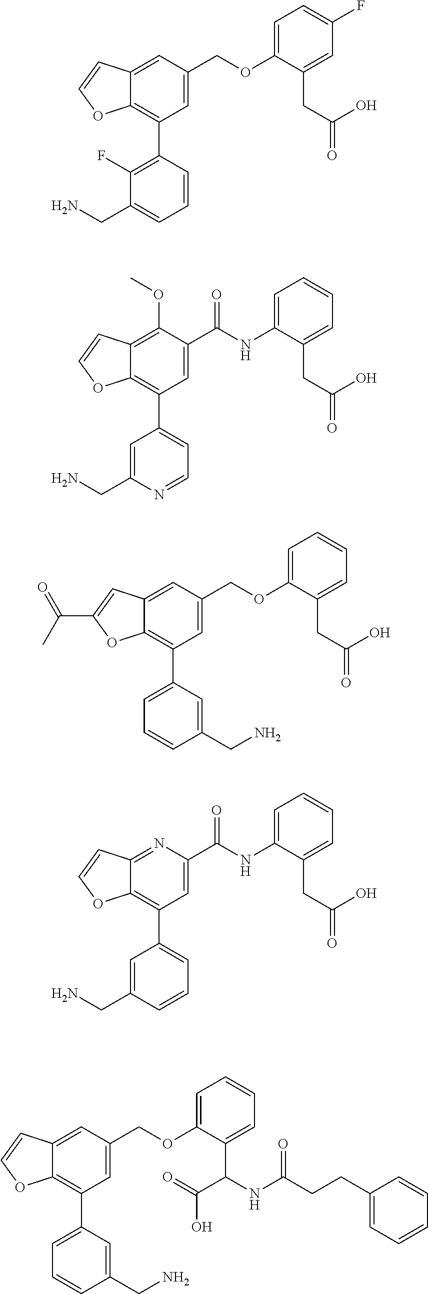 [SMILES: NCc1cccc(-c2cc(COc3ccc(F)cc3CC(=O)O)cc3ccoc23)c1F ; COc1c(C(=O)Nc2ccccc2CC(=O)O)cc(-c2ccnc(CN)c2)c2occc12 ; CC(=O)c1cc2cc(COc3ccccc3CC(=O)O)cc(-c3cccc(CN)c3)c2o1 ; NCc1cccc(-c2cc(C(=O)Nc3ccccc3CC(=O)O)nc3ccoc23)c1 ; NCc1cccc(-c2cc(COc3ccccc3C(NC(=O)CCc3ccccc3)C(=O)O)cc3ccoc23)c1]

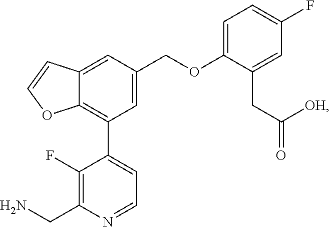 [SMILES: NCc1nccc(-c2cc(COc3ccc(F)cc3CC(=O)O)cc3ccoc23)c1F]

or a pharmaceutically acceptable salt or prodrug thereof; and a pharmaceutically acceptable carrier. In certain embodiments, the oral dosage form is a capsule.

In further aspects, the invention provides methods of treating or preventing a disease or condition characterized by aberrant complement system activity, comprising orally administering to a patient in need thereof a therapeutically effective amount of a compound disclosed herein, or a pharmaceutically acceptable salt or prodrug thereof. In certain embodiments, the disease or condition characterized by aberrant complement system activity is an immunological disorder. In certain embodiments, the disease or condition characterized by aberrant complement system activity is a disease of the central nervous system. In certain embodiments, the disease or condition characterized by aberrant complement system activity is a neurodegenerative disease or neurological disease. In certain embodiments, the disease or condition characterized by aberrant complement system activity is a renal disease. In certain embodiments, the disease or condition characterized by aberrant complement system activity is a cardiovascular disease. In certain embodiments, the disease or condition characterized by aberrant complement system activity is selected from the group consisting of paroxysmal nocturnal hemoglobinuria (PNH), atypical hemolytic uremic syndrome, organ transplant rejection, myasthenia gravis, neuromyelitis optica, membranoproliferative glomerulonephritis, dense-deposit disease, cold agglutinin disease, and catastrophic antiphospholipid syndrome. In certain embodiments, the disease or condition characterized by aberrant complement system activity is PNH.

### BRIEF DESCRIPTION OF THE DRAWINGS

FIG. 1 shows that a compound of the invention exhibits dose proportionality in plasma concentration-time curves following oral administration of single escalating doses from 10 mg to 1200 mg in Part 1 in healthy subjects (arithmetic mean±SD).

FIG. 2 shows that exposure (Cₘₐₓ) to a compound of the invention across the single ascending oral dose cohorts of Part 1 was approximately linear and dose-proportional.

FIG. 3 shows that exposure [AUC₍₀₋₂₄₎] to a compound of the invention across the single ascending oral dose cohorts of Part 1 was approximately linear and dose-proportional.

FIG. 4 shows dose-dependent inhibition of AP Wieslab activity was detected following oral administration of single ascending dose from 10 mg to 1200 mg of a compound of the invention in healthy subjects in Part 1. A >90% suppression of AP activity that was sustained for at least 12 hours post-dose was observed with a single 100 mg or higher oral dose of a compound of the invention.

FIG. 5 shows dose-dependent inhibition of AP hemolysis was detected following oral administration of single ascending doses from 10 mg to 1200 mg of a compound of the invention in healthy subjects in Part 1. A >90% suppression of AP activity that was sustained for at least 12 hours post-dose was observed with a single 100 mg or higher oral dose of a compound of the invention.

FIG. 6 shows steady state PK and PD (AP Wieslab) profiles 12 hours post-dose on Day 3 (Cohorts 4 and 5) or Day 7 (Cohorts 1 and 2) following oral administration of multiple ascending doses from 100 mg/day to 800 mg/day of a compound of the invention in healthy subjects in Part 2. Dose-dependent exposure and dose-dependent inhibition of AP Wieslab activity were observed. A >90% inhibition of AP activity was observed for all multiple ascending doses for at least 12 hours following administration of the final dose.

FIG. 7A shows that exposure (Cₘₐₓ) to a compound of the invention across the multiple ascending oral dose cohorts of Part 2 (Cohorts 1, 2, 4, and 5) was approximately linear and dose-proportional.

FIG. 7B shows that exposure (AUCₜₐᵤ) to a compound of the invention across the multiple ascending oral dose cohorts of Part 2 (Cohorts 1, 2, 4, and 5) was approximately linear and dose-proportional.

FIG. 8A shows the individual (n=10) PD profiles of a compound of the invention (AP Wieslab activity) in healthy subjects through 24 hours post-dose on Day 7 for Cohort 1 of Part 2.

FIG. 8B shows the individual (n=10) PD profiles of a compound of the invention (AP Wieslab activity) in healthy subjects through 24 hours post-dose on Day 7 for Cohort 2 of Part 2.

FIG. 8C shows the individual (n=10) PD profiles of a compound of the invention (AP Wieslab activity) in healthy subjects through 24 hours post-dose on Day 3 for Cohort 4 of Part 2.

FIG. 8D shows the individual (n=10) PD profiles of a compound of the invention (AP Wieslab activity) in healthy subjects through 24 hours post-dose on Day 3 for Cohort 5 of Part 2.

FIG. 8E shows steady state PD (AP Wieslab) profiles 24 hours post-dose on Day 3 (Cohorts 4 and 5) or Day 7 (Cohorts 1 and 2) following oral administration of multiple ascending doses from 100 mg/day to 800 mg/day of a compound of the invention in healthy subjects in Part 2. A >90% inhibition of AP activity was observed for all multiple ascending doses for at least 12 hours following administration of the final dose.

FIG. 9A shows the individual (n=10) PD profiles of a compound of the invention (AP hemolysis) in healthy subjects through 24 hours post-dose on Day 7 for Cohort 1 of Part 2.

FIG. 9B shows the individual (n=10) PD profiles of a compound of the invention (AP hemolysis) in healthy subjects through 24 hours post-dose on Day 7 for Cohort 2 of Part 2.

FIG. 9C shows the individual (n=10) PD profiles of a compound of the invention (AP hemolysis) in healthy subjects through 24 hours post-dose on Day 3 for Cohort 4 of Part 2.

FIG. 9D shows the individual (n=10) PD profiles of a compound of the invention (AP hemolysis) in healthy subjects through 24 hours post-dose on Day 3 for Cohort 5 of Part 2.

FIG. 9E shows steady state PD (AP hemolysis) profiles 24 hours post-dose on Day 3 (Cohorts 4 and 5) or Day 7 (Cohorts 1 and 2) following oral administration of multiple ascending doses from 100 mg/day to 800 mg/day of a compound of the invention in healthy subjects in Part 2. A >90% inhibition of AP activity was observed for all multiple ascending doses for at least 12 hours following administration of the final dose.

FIG. 10 shows the PD profile of a compound of the invention (AP hemolysis and AP Wieslab assay) in healthy subjects at 24 hour (Day 1) PK troughs and Day 3 (Cohorts 4 and 5) or Day 7 (Cohorts 1 and 2) PK troughs for Cohorts 1, 2, 4, and 5 of Part 2.

FIG. 11A shows the PD profile of a compound of the invention (lactate dehydrogenase; upper limit of normal, ULN) through 28 days of dosing in PNH subjects naïve to C5-inhibitor treatment (Cohort 1 of Part 3A).

FIG. 11B shows the PD profile of a compound of the invention (absolute reticulocyte count; 10³/μL) through 28 days of dosing in PNH subjects naïve to C5-inhibitor treatment (Cohort 1 of Part 3A).

FIG. 11C shows the PD profile of a compound of the invention (total bilirubin; upper limit of normal, ULN) through 28 days of dosing in PNH subjects naïve to C5-inhibitor treatment (Cohort 1 of Part 3A).

FIG. 11D shows the PD profile of a compound of the invention (hemoglobin levels in g/dL) through 28 days of dosing in PNH subjects naïve to C5-inhibitor treatment (Cohort 1 of Part 3A).

FIG. 11E shows the PD profile of a compound of the invention (PNH clone size; percent change) through 28 days of dosing in PNH subjects naïve to C5-inhibitor treatment (Cohort 1 of Part 3A).

### DETAILED DESCRIPTION

Inhibitors of the complement system are useful in therapeutic methods and compositions suitable for use in treating disorders of the immune, renal, cardiovascular, and neurological systems. The compounds disclosed herein are novel, potent, selective, and orally bioavailable small-molecule inhibitors of human factor D. In preclinical in vitro studies, compounds of the invention suppressed the AP-mediated hemolysis of erythrocytes from patients with PNH and blocked AP-mediated C3 fragment deposition on PNH erythrocytes at low nanomolar drug concentrations. Oral dosing of compounds of the invention achieved >95% suppression of AP mediated hemolysis in preclinical in vivo studies of complement activity in monkeys. Therefore, targeting proximal complement inhibition by compounds of the invention has the potential to prevent intravascular and avoid extravascular hemolysis associated with C3 fragment deposition specifically caused by C5 inhibition in patients with PNH.

### Definitions

The articles “a” and “an” are used herein to refer to one or to more than one (i.e., to at least one) of the grammatical object of the article. By way of example, “an element” means one element or more than one element.

Unless otherwise defined, all technical and scientific terms used herein have the same meaning as commonly understood by one of ordinary skill in the art to which this invention belongs. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000). Although methods and materials similar or equivalent to those described herein can be used in the practice or testing of the present invention, suitable methods and materials are described below.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of a compound of the invention. As another example, the salt may comprise less than one inorganic or organic acid molecule per molecule of base, such as two molecules of a compound of the invention per molecule of tartaric acid.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

All of the above, and any other publications, patents, published patent applications, or other references referred to in this application are specifically incorporated by reference herein. In case of conflict, the present specification, including its specific definitions, will control. In addition, the materials, methods, and examples are illustrative only and not intended to be limiting.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

As used herein, a therapeutic that “prevents” or “reduces the risk of developing” a disease, disorder, or condition refers to a compound that, in a statistical sample, reduces the occurrence of the disease, disorder, or condition in the treated sample relative to an untreated control sample, or delays the onset or reduces the severity of one or more symptoms of the disorder or condition relative to the untreated control sample.

In certain embodiments, compounds of the invention may be used alone or conjointly administered with another therapeutic agent. The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

The term “treating” includes prophylactic and/or therapeutic treatments. The term “prophylactic or therapeutic” treatment is art-recognized and includes administration to the subject of one or more of the disclosed compositions. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic (i.e., it is intended to diminish, ameliorate, or stabilize the existing unwanted condition or side effects thereof).

The term “prodrug” is intended to encompass compounds which, under physiologic conditions, are converted into therapeutically active agents. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) and esters or amides of phosphates and phosphonic acids are preferred prodrugs of the present invention.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

### Oral Dosage Forms of the Invention

One aspect of the invention provides oral dosage forms of small molecules that inhibit human factor D.

In some embodiments, the oral dosage form comprises a compound selected from:

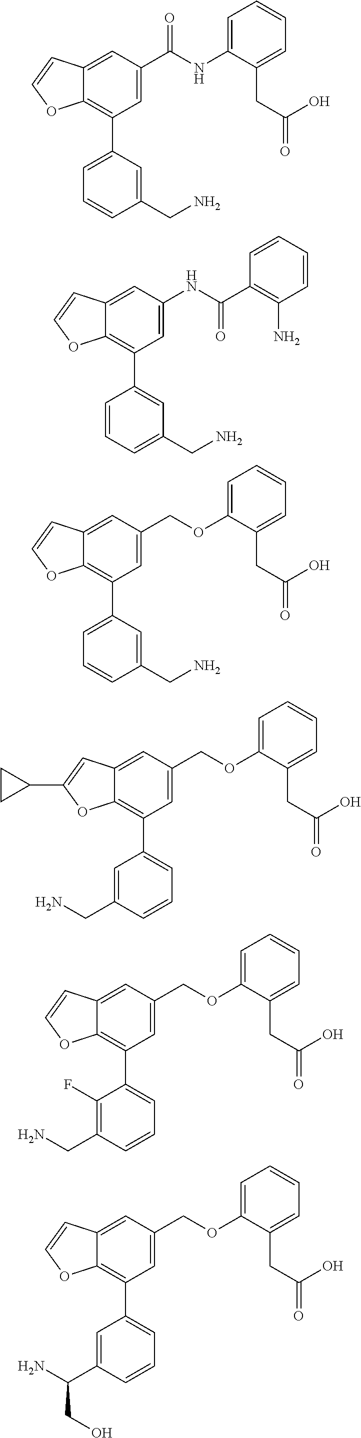

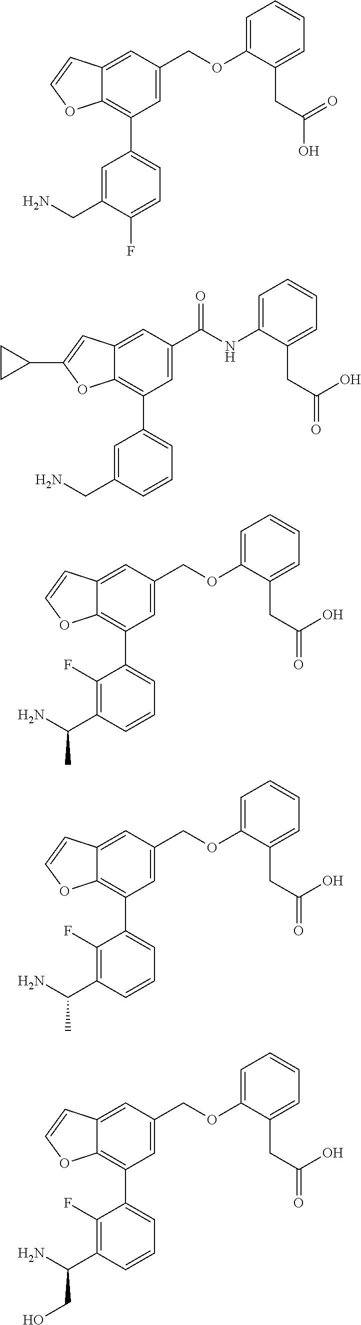 [SMILES: NCc1cc(-c2cc(COc3ccccc3CC(=O)O)cc3ccoc23)ccc1F ; NCc1cccc(-c2cc(C(=O)Nc3ccccc3CC(=O)O)cc3cc(C4CC4)oc23)c1 ; C[C@@H](N)c1cccc(-c2cc(COc3ccccc3CC(=O)O)cc3ccoc23)c1F ; C[C@H](N)c1cccc(-c2cc(COc3ccccc3CC(=O)O)cc3ccoc23)c1F ; N[C@H](CO)c1cccc(-c2cc(COc3ccccc3CC(=O)O)cc3ccoc23)c1F]

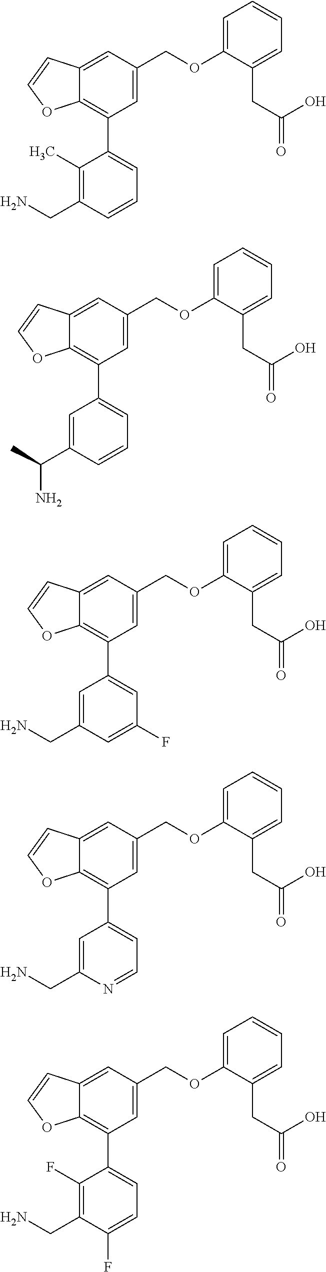 [SMILES: Cc1c(CN)cccc1-c1cc(COc2ccccc2CC(=O)O)cc2ccoc12 ; C[C@H](N)c1cccc(-c2cc(COc3ccccc3CC(=O)O)cc3ccoc23)c1 ; NCc1cc(F)cc(-c2cc(COc3ccccc3CC(=O)O)cc3ccoc23)c1 ; NCc1cc(-c2cc(COc3ccccc3CC(=O)O)cc3ccoc23)ccn1 ; NCc1c(F)ccc(-c2cc(COc3ccccc3CC(=O)O)cc3ccoc23)c1F]

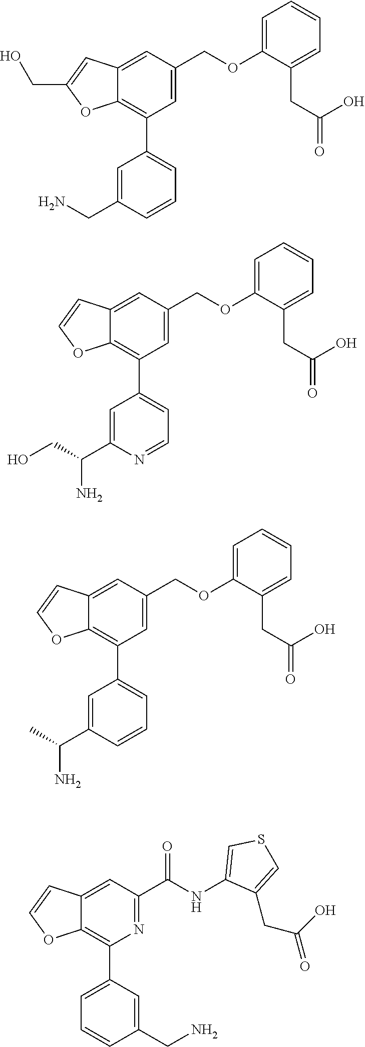 [SMILES: NCc1cccc(-c2cc(COc3ccccc3CC(=O)O)cc3cc(CO)oc23)c1 ; N[C@H](CO)c1cc(-c2cc(COc3ccccc3CC(=O)O)cc3ccoc23)ccn1 ; C[C@@H](N)c1cccc(-c2cc(COc3ccccc3CC(=O)O)cc3ccoc23)c1 ; NCc1cccc(-c2nc(C(=O)Nc3cscc3CC(=O)O)cc3ccoc23)c1]

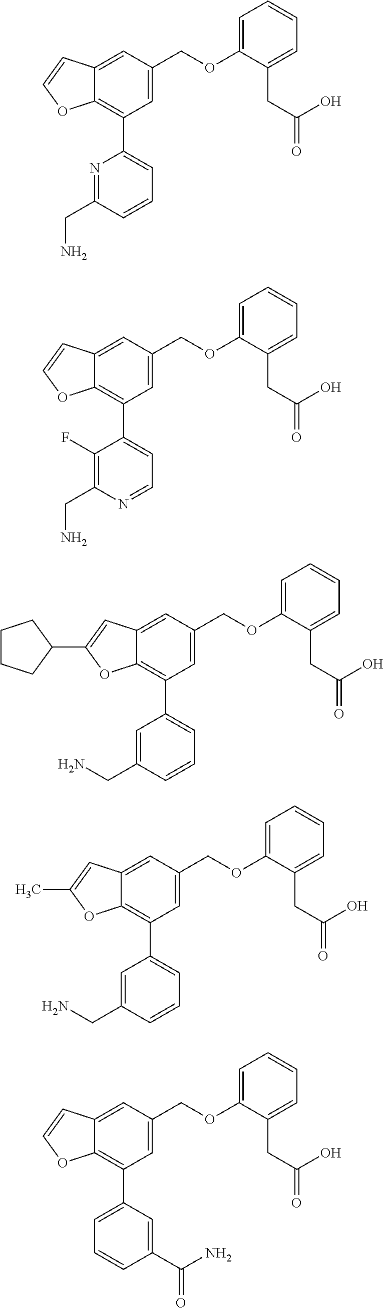 [SMILES: NCc1cccc(-c2cc(COc3ccccc3CC(=O)O)cc3ccoc23)n1 ; NCc1nccc(-c2cc(COc3ccccc3CC(=O)O)cc3ccoc23)c1F ; NCc1cccc(-c2cc(COc3ccccc3CC(=O)O)cc3cc(C4CCCC4)oc23)c1 ; Cc1cc2cc(COc3ccccc3CC(=O)O)cc(-c3cccc(CN)c3)c2o1 ; NC(=O)c1cccc(-c2cc(COc3ccccc3CC(=O)O)cc3ccoc23)c1]

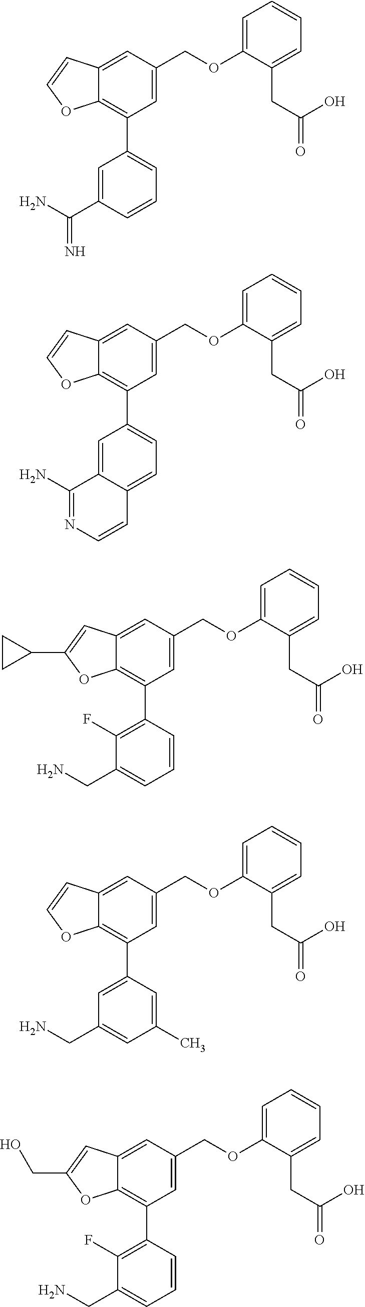 [SMILES: N=C(N)c1cccc(-c2cc(COc3ccccc3CC(=O)O)cc3ccoc23)c1 ; Nc1nccc2ccc(-c3cc(COc4ccccc4CC(=O)O)cc4ccoc34)cc12 ; NCc1cccc(-c2cc(COc3ccccc3CC(=O)O)cc3cc(C4CC4)oc23)c1F ; Cc1cc(CN)cc(-c2cc(COc3ccccc3CC(=O)O)cc3ccoc23)c1 ; NCc1cccc(-c2cc(COc3ccccc3CC(=O)O)cc3cc(CO)oc23)c1F]

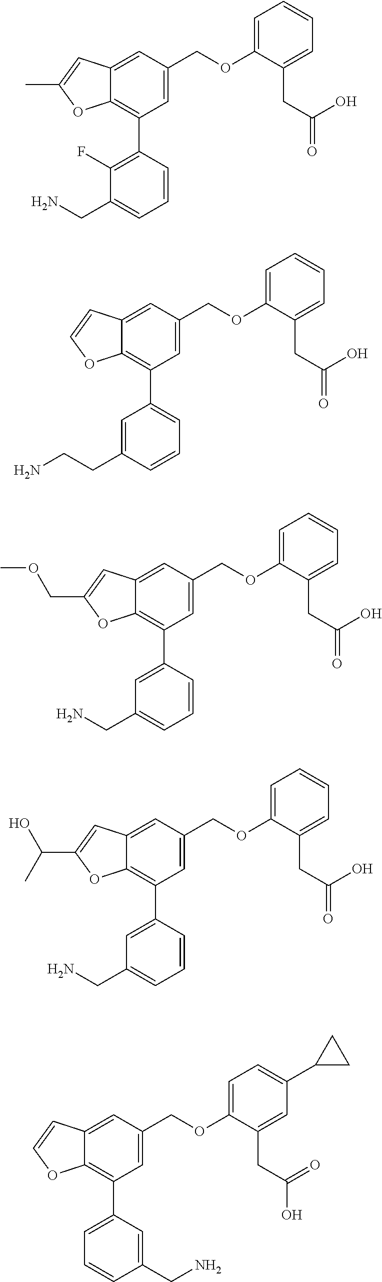 [SMILES: Cc1cc2cc(COc3ccccc3CC(=O)O)cc(-c3cccc(CN)c3F)c2o1 ; NCCc1cccc(-c2cc(COc3ccccc3CC(=O)O)cc3ccoc23)c1 ; COCc1cc2cc(COc3ccccc3CC(=O)O)cc(-c3cccc(CN)c3)c2o1 ; CC(O)c1cc2cc(COc3ccccc3CC(=O)O)cc(-c3cccc(CN)c3)c2o1 ; NCc1cccc(-c2cc(COc3ccc(C4CC4)cc3CC(=O)O)cc3ccoc23)c1]

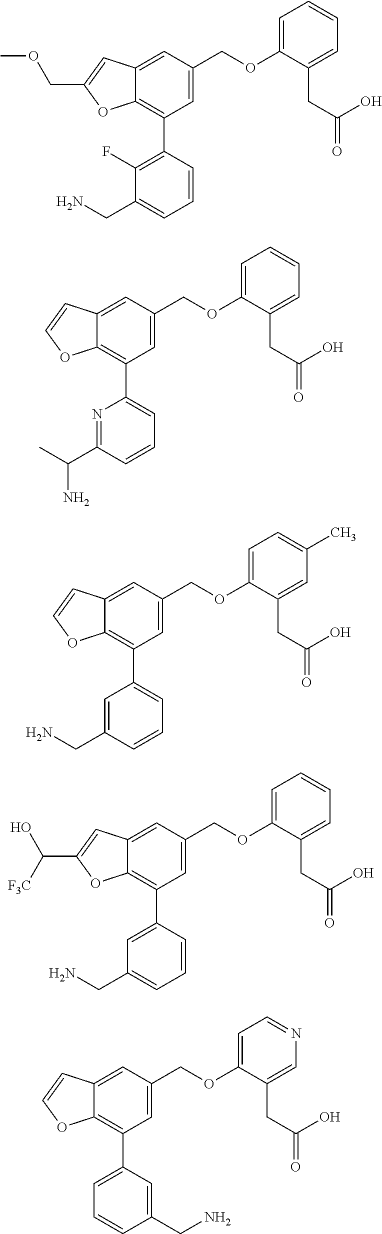 [SMILES: COCc1cc2cc(COc3ccccc3CC(=O)O)cc(-c3cccc(CN)c3F)c2o1 ; CC(N)c1cccc(-c2cc(COc3ccccc3CC(=O)O)cc3ccoc23)n1 ; Cc1ccc(OCc2cc(-c3cccc(CN)c3)c3occc3c2)c(CC(=O)O)c1 ; NCc1cccc(-c2cc(COc3ccccc3CC(=O)O)cc3cc(C(O)C(F)(F)F)oc23)c1 ; NCc1cccc(-c2cc(COc3ccncc3CC(=O)O)cc3ccoc23)c1]

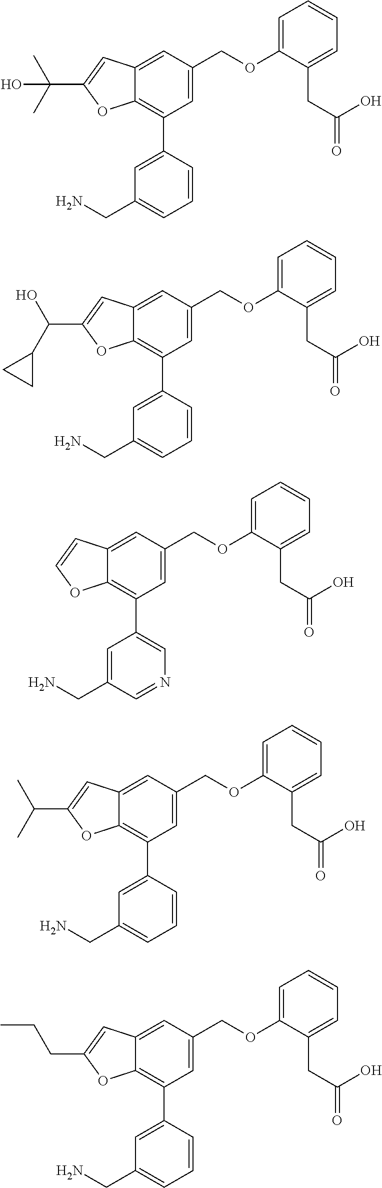 [SMILES: CC(C)(O)c1cc2cc(COc3ccccc3CC(=O)O)cc(-c3cccc(CN)c3)c2o1 ; NCc1cccc(-c2cc(COc3ccccc3CC(=O)O)cc3cc(C(O)C4CC4)oc23)c1 ; NCc1cncc(-c2cc(COc3ccccc3CC(=O)O)cc3ccoc23)c1 ; CC(C)c1cc2cc(COc3ccccc3CC(=O)O)cc(-c3cccc(CN)c3)c2o1 ; CCCc1cc2cc(COc3ccccc3CC(=O)O)cc(-c3cccc(CN)c3)c2o1]

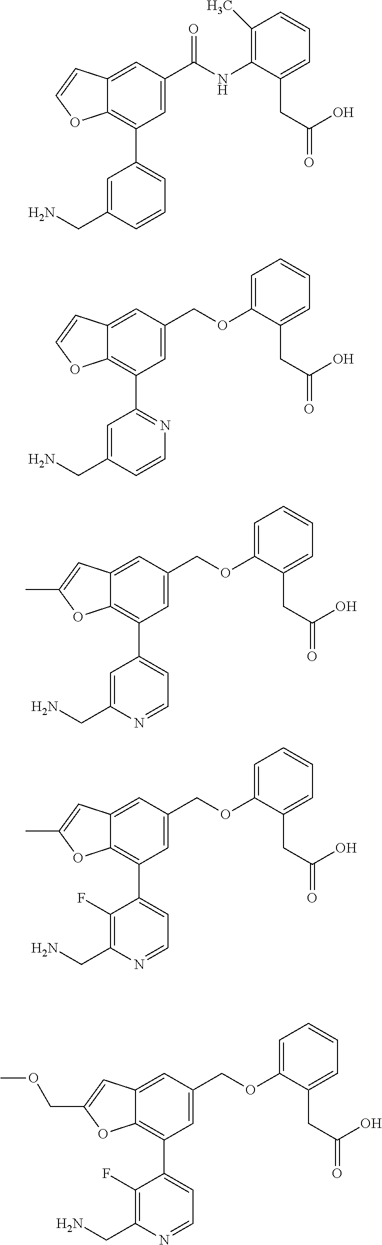 [SMILES: Cc1cccc(CC(=O)O)c1NC(=O)c1cc(-c2cccc(CN)c2)c2occc2c1 ; NCc1ccnc(-c2cc(COc3ccccc3CC(=O)O)cc3ccoc23)c1 ; Cc1cc2cc(COc3ccccc3CC(=O)O)cc(-c3ccnc(CN)c3)c2o1 ; Cc1cc2cc(COc3ccccc3CC(=O)O)cc(-c3ccnc(CN)c3F)c2o1 ; COCc1cc2cc(COc3ccccc3CC(=O)O)cc(-c3ccnc(CN)c3F)c2o1]

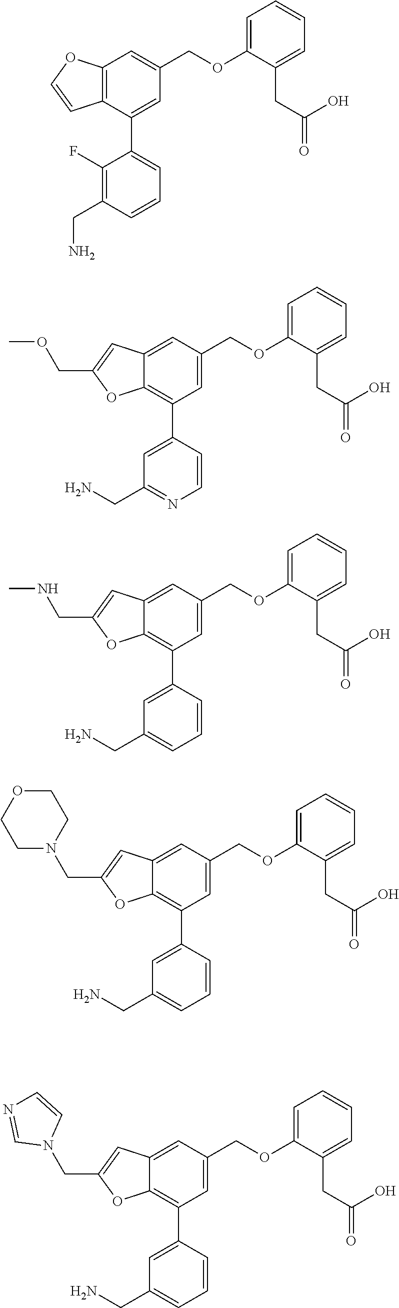 [SMILES: NCc1cccc(-c2cc(COc3ccccc3CC(=O)O)cc3occc23)c1F ; COCc1cc2cc(COc3ccccc3CC(=O)O)cc(-c3ccnc(CN)c3)c2o1 ; CNCc1cc2cc(COc3ccccc3CC(=O)O)cc(-c3cccc(CN)c3)c2o1 ; NCc1cccc(-c2cc(COc3ccccc3CC(=O)O)cc3cc(CN4CCOCC4)oc23)c1 ; NCc1cccc(-c2cc(COc3ccccc3CC(=O)O)cc3cc(Cn4ccnc4)oc23)c1]

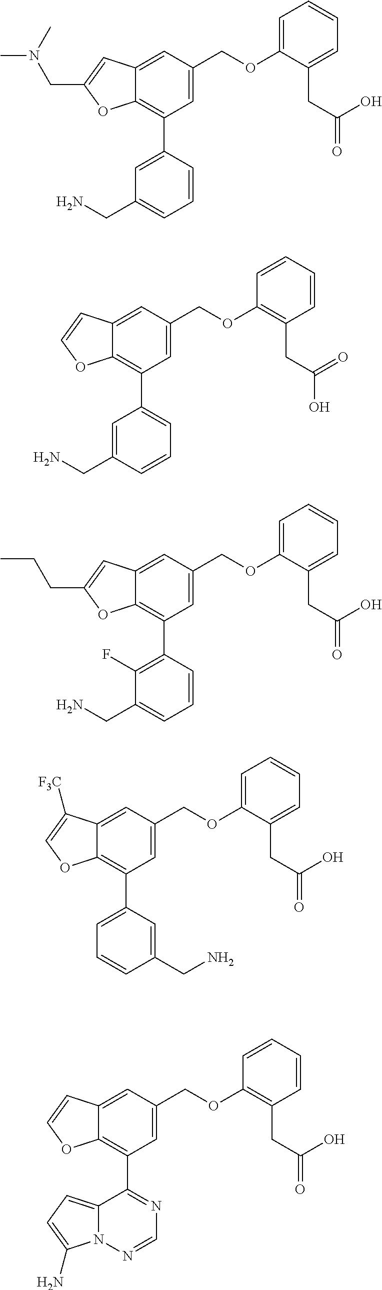 [SMILES: CN(C)Cc1cc2cc(COc3ccccc3CC(=O)O)cc(-c3cccc(CN)c3)c2o1 ; NCc1cccc(-c2cc(COc3ccccc3CC(=O)O)cc3ccoc23)c1 ; CCCc1cc2cc(COc3ccccc3CC(=O)O)cc(-c3cccc(CN)c3F)c2o1 ; NCc1cccc(-c2cc(COc3ccccc3CC(=O)O)cc3c(C(F)(F)F)coc23)c1 ; Nc1ccc2c(-c3cc(COc4ccccc4CC(=O)O)cc4ccoc34)ncnn12]

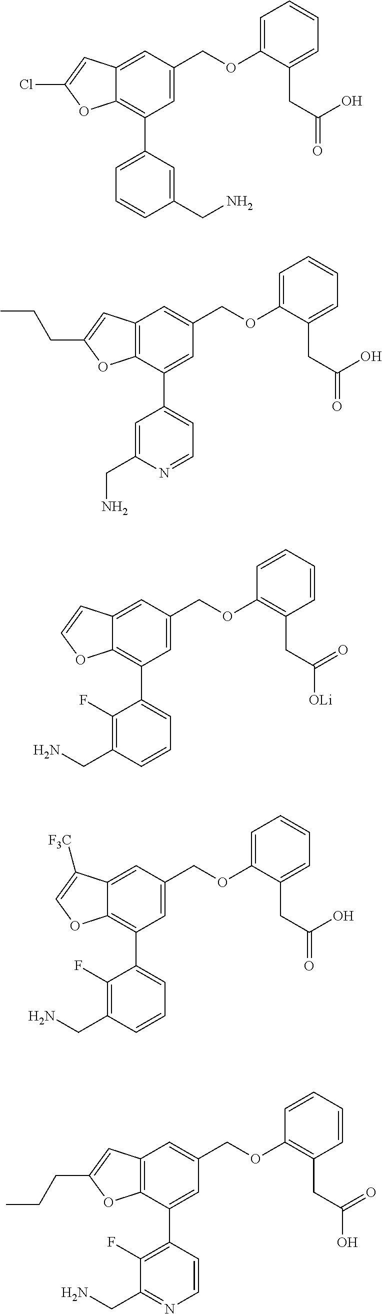

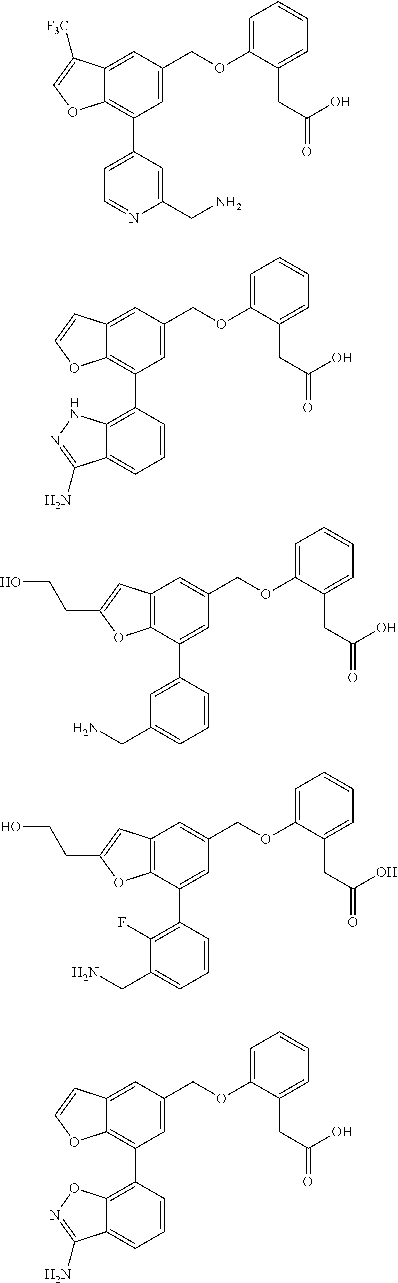 [SMILES: NCc1cc(-c2cc(COc3ccccc3CC(=O)O)cc3c(C(F)(F)F)coc23)ccn1 ; Nc1n[nH]c2c(-c3cc(COc4ccccc4CC(=O)O)cc4ccoc34)cccc12 ; NCc1cccc(-c2cc(COc3ccccc3CC(=O)O)cc3cc(CCO)oc23)c1 ; NCc1cccc(-c2cc(COc3ccccc3CC(=O)O)cc3cc(CCO)oc23)c1F ; Nc1noc2c(-c3cc(COc4ccccc4CC(=O)O)cc4ccoc34)cccc12]

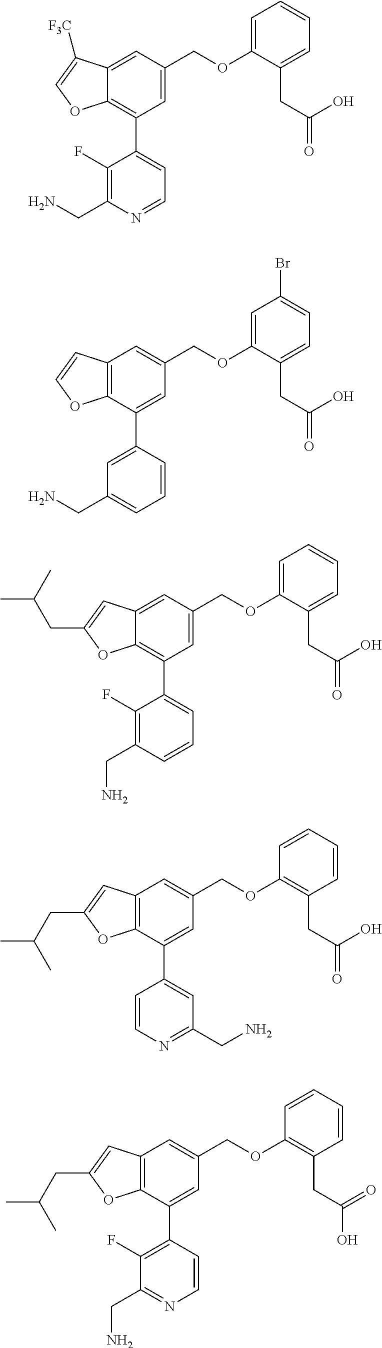 [SMILES: NCc1nccc(-c2cc(COc3ccccc3CC(=O)O)cc3c(C(F)(F)F)coc23)c1F ; NCc1cccc(-c2cc(COc3cc(Br)ccc3CC(=O)O)cc3ccoc23)c1 ; CC(C)Cc1cc2cc(COc3ccccc3CC(=O)O)cc(-c3cccc(CN)c3F)c2o1 ; CC(C)Cc1cc2cc(COc3ccccc3CC(=O)O)cc(-c3ccnc(CN)c3)c2o1 ; CC(C)Cc1cc2cc(COc3ccccc3CC(=O)O)cc(-c3ccnc(CN)c3F)c2o1]

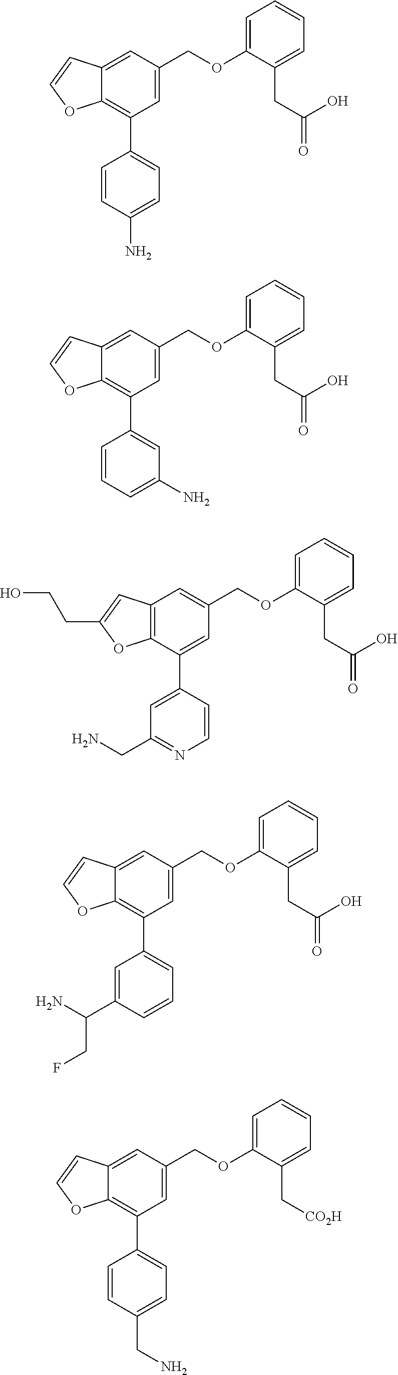 [SMILES: Nc1ccc(-c2cc(COc3ccccc3CC(=O)O)cc3ccoc23)cc1 ; Nc1cccc(-c2cc(COc3ccccc3CC(=O)O)cc3ccoc23)c1 ; NCc1cc(-c2cc(COc3ccccc3CC(=O)O)cc3cc(CCO)oc23)ccn1 ; NC(CF)c1cccc(-c2cc(COc3ccccc3CC(=O)O)cc3ccoc23)c1 ; NCc1ccc(-c2cc(COc3ccccc3CC(=O)O)cc3ccoc23)cc1]

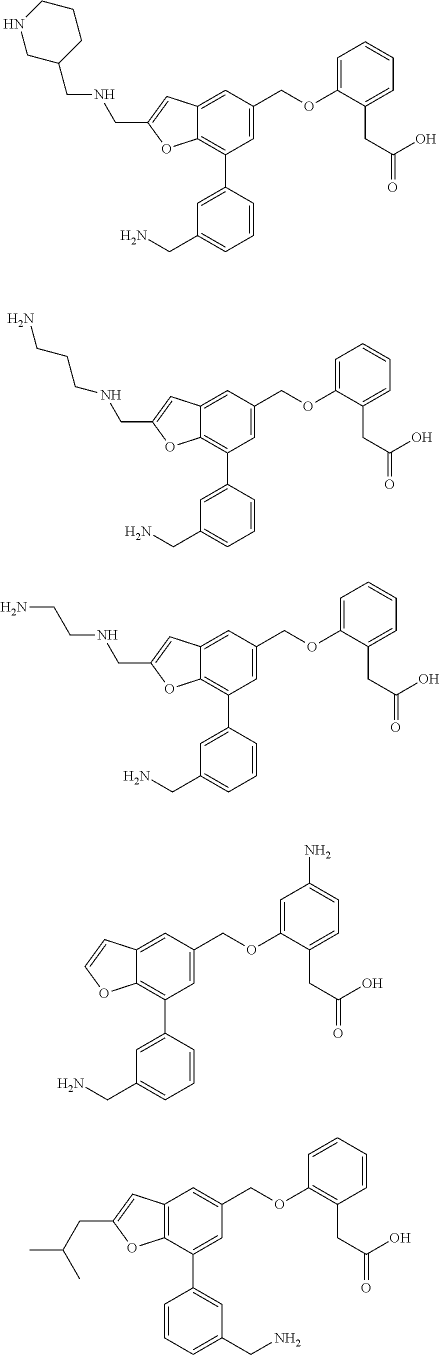 [SMILES: NCc1cccc(-c2cc(COc3ccccc3CC(=O)O)cc3cc(CNCC4CCCNC4)oc23)c1 ; NCCCNCc1cc2cc(COc3ccccc3CC(=O)O)cc(-c3cccc(CN)c3)c2o1 ; NCCNCc1cc2cc(COc3ccccc3CC(=O)O)cc(-c3cccc(CN)c3)c2o1 ; NCc1cccc(-c2cc(COc3cc(N)ccc3CC(=O)O)cc3ccoc23)c1 ; CC(C)Cc1cc2cc(COc3ccccc3CC(=O)O)cc(-c3cccc(CN)c3)c2o1]

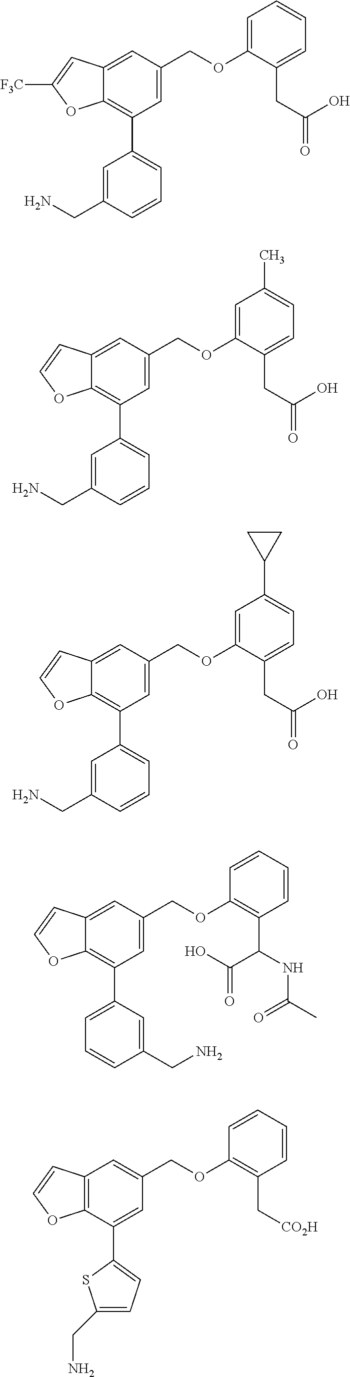 [SMILES: NCc1cccc(-c2cc(COc3ccccc3CC(=O)O)cc3cc(C(F)(F)F)oc23)c1 ; Cc1ccc(CC(=O)O)c(OCc2cc(-c3cccc(CN)c3)c3occc3c2)c1 ; NCc1cccc(-c2cc(COc3cc(C4CC4)ccc3CC(=O)O)cc3ccoc23)c1 ; CC(=O)NC(C(=O)O)c1ccccc1OCc1cc(-c2cccc(CN)c2)c2occc2c1 ; NCc1ccc(-c2cc(COc3ccccc3CC(=O)O)cc3ccoc23)s1]

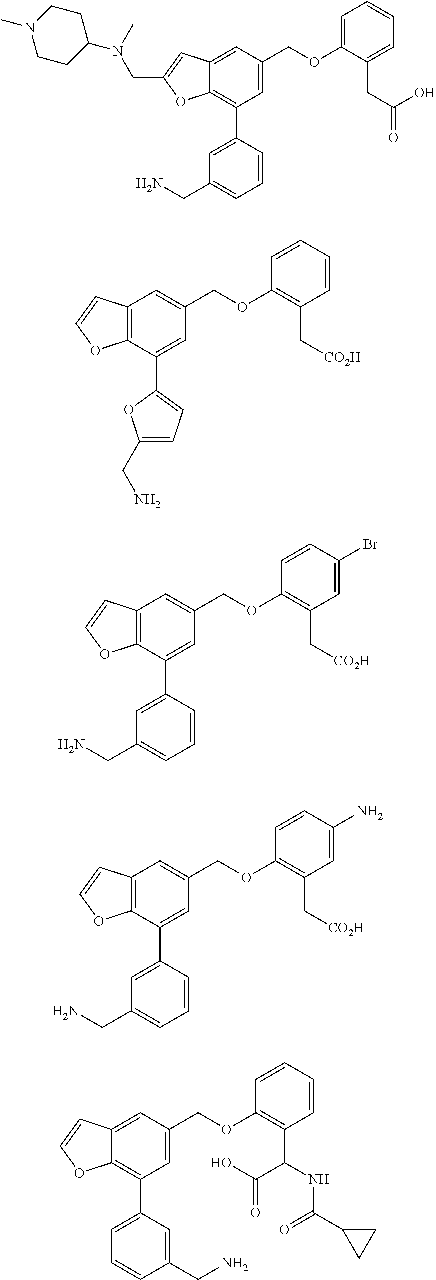 [SMILES: CN1CCC(N(C)Cc2cc3cc(COc4ccccc4CC(=O)O)cc(-c4cccc(CN)c4)c3o2)CC1 ; NCc1ccc(-c2cc(COc3ccccc3CC(=O)O)cc3ccoc23)o1 ; NCc1cccc(-c2cc(COc3ccc(Br)cc3CC(=O)O)cc3ccoc23)c1 ; NCc1cccc(-c2cc(COc3ccc(N)cc3CC(=O)O)cc3ccoc23)c1 ; NCc1cccc(-c2cc(COc3ccccc3C(NC(=O)C3CC3)C(=O)O)cc3ccoc23)c1]

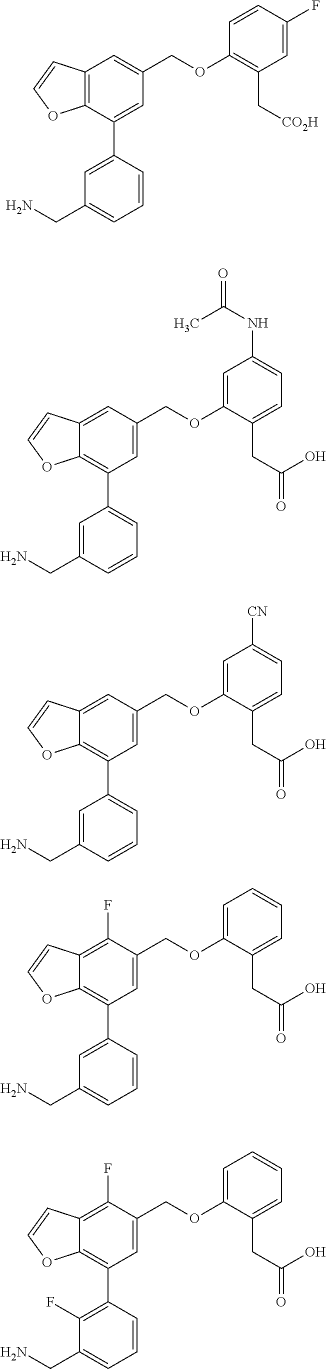

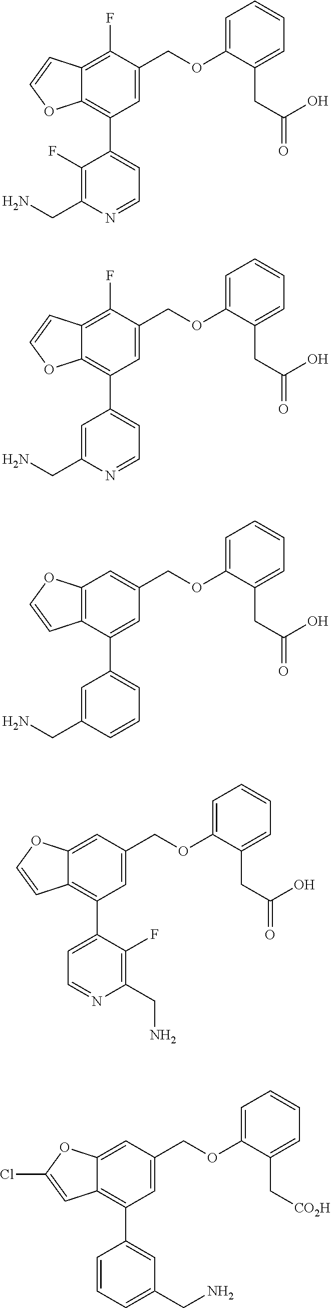 [SMILES: NCc1nccc(-c2cc(COc3ccccc3CC(=O)O)c(F)c3ccoc23)c1F ; NCc1cc(-c2cc(COc3ccccc3CC(=O)O)c(F)c3ccoc23)ccn1 ; NCc1cccc(-c2cc(COc3ccccc3CC(=O)O)cc3occc23)c1 ; NCc1nccc(-c2cc(COc3ccccc3CC(=O)O)cc3occc23)c1F ; NCc1cccc(-c2cc(COc3ccccc3CC(=O)O)cc3oc(Cl)cc23)c1]

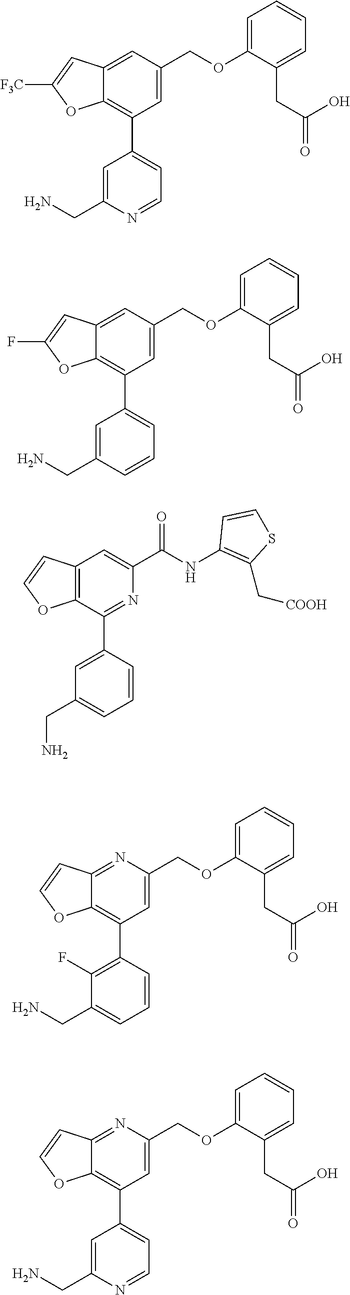 [SMILES: NCc1cc(-c2cc(COc3ccccc3CC(=O)O)cc3cc(C(F)(F)F)oc23)ccn1 ; NCc1cccc(-c2cc(COc3ccccc3CC(=O)O)cc3cc(F)oc23)c1 ; NCc1cccc(-c2nc(C(=O)Nc3ccsc3CC(=O)O)cc3ccoc23)c1 ; NCc1cccc(-c2cc(COc3ccccc3CC(=O)O)nc3ccoc23)c1F ; NCc1cc(-c2cc(COc3ccccc3CC(=O)O)nc3ccoc23)ccn1]

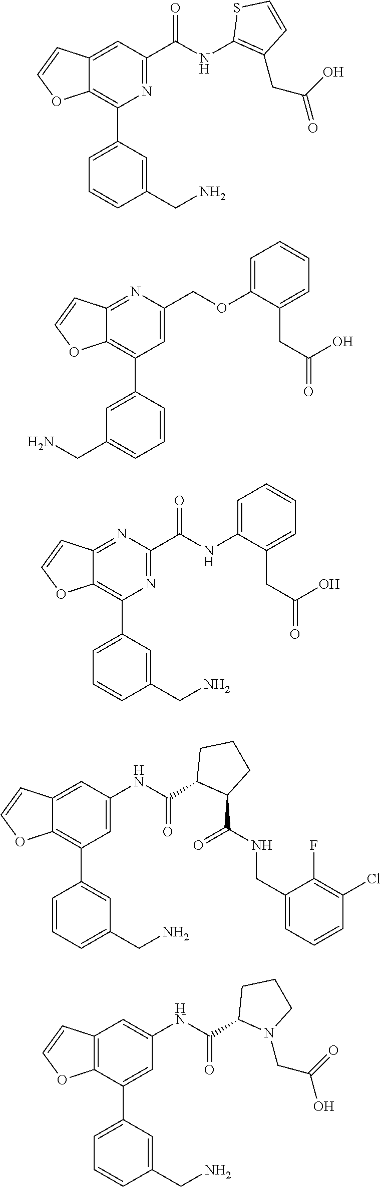 [SMILES: NCc1cccc(-c2nc(C(=O)Nc3sccc3CC(=O)O)cc3ccoc23)c1 ; NCc1cccc(-c2cc(COc3ccccc3CC(=O)O)nc3ccoc23)c1 ; NCc1cccc(-c2nc(C(=O)Nc3ccccc3CC(=O)O)nc3ccoc23)c1 ; NCc1cccc(-c2cc(NC(=O)[C@@H]3CCC[C@H]3C(=O)NCc3cccc(Cl)c3F)cc3ccoc23)c1 ; NCc1cccc(-c2cc(NC(=O)[C@@H]3CCCN3CC(=O)O)cc3ccoc23)c1]

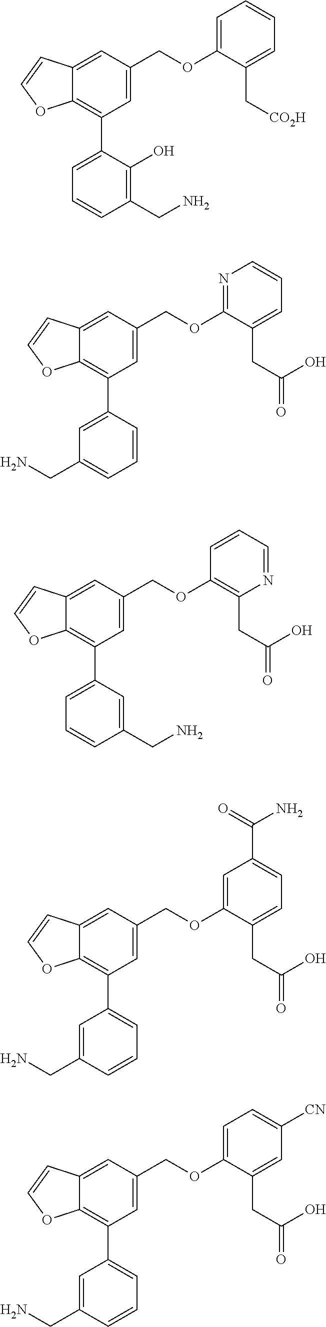 [SMILES: NCc1cccc(-c2cc(COc3ccccc3CC(=O)O)cc3ccoc23)c1O ; NCc1cccc(-c2cc(COc3ncccc3CC(=O)O)cc3ccoc23)c1 ; NCc1cccc(-c2cc(COc3cccnc3CC(=O)O)cc3ccoc23)c1 ; NCc1cccc(-c2cc(COc3cc(C(N)=O)ccc3CC(=O)O)cc3ccoc23)c1 ; N#Cc1ccc(OCc2cc(-c3cccc(CN)c3)c3occc3c2)c(CC(=O)O)c1]

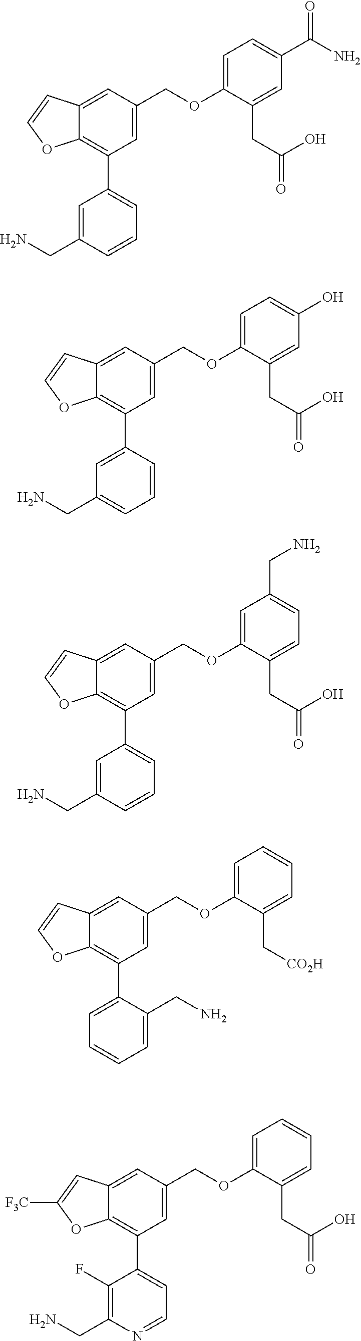 [SMILES: NCc1cccc(-c2cc(COc3ccc(C(N)=O)cc3CC(=O)O)cc3ccoc23)c1 ; NCc1cccc(-c2cc(COc3ccc(O)cc3CC(=O)O)cc3ccoc23)c1 ; NCc1cccc(-c2cc(COc3cc(CN)ccc3CC(=O)O)cc3ccoc23)c1 ; NCc1ccccc1-c1cc(COc2ccccc2CC(=O)O)cc2ccoc12 ; NCc1nccc(-c2cc(COc3ccccc3CC(=O)O)cc3cc(C(F)(F)F)oc23)c1F]

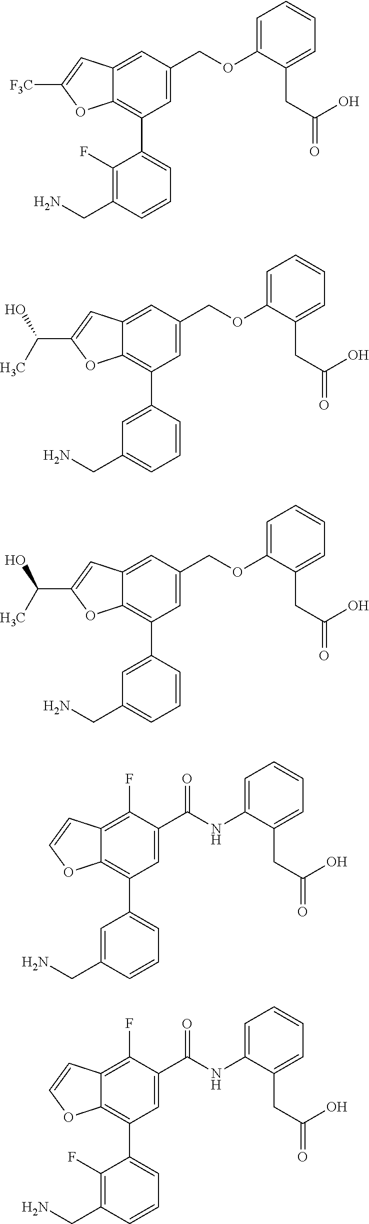

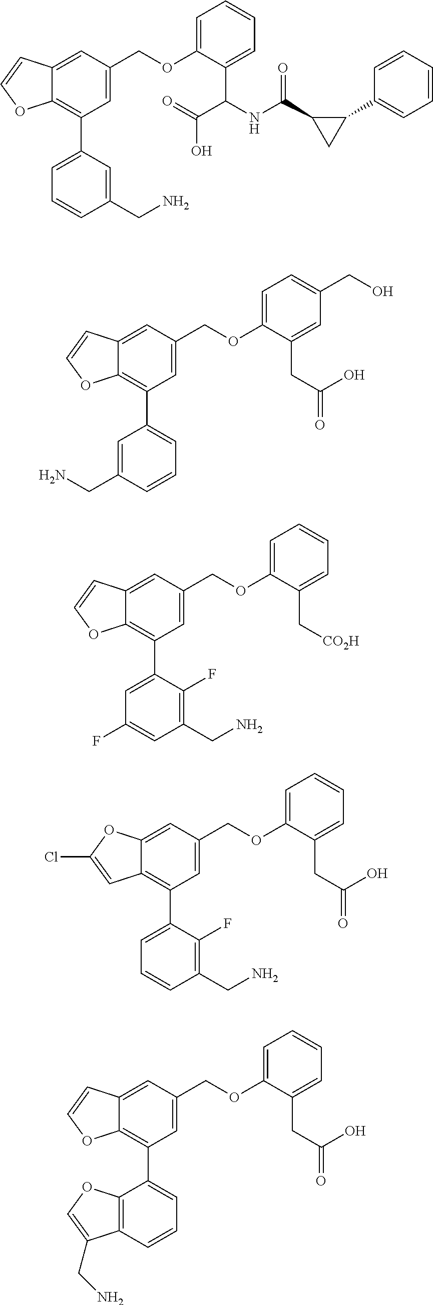 [SMILES: NCc1cccc(-c2cc(COc3ccccc3C(NC(=O)[C@@H]3C[C@H]3c3ccccc3)C(=O)O)cc3ccoc23)c1 ; NCc1cccc(-c2cc(COc3ccc(CO)cc3CC(=O)O)cc3ccoc23)c1 ; NCc1cc(F)cc(-c2cc(COc3ccccc3CC(=O)O)cc3ccoc23)c1F ; NCc1cccc(-c2cc(COc3ccccc3CC(=O)O)cc3oc(Cl)cc23)c1F ; NCc1coc2c(-c3cc(COc4ccccc4CC(=O)O)cc4ccoc34)cccc12]

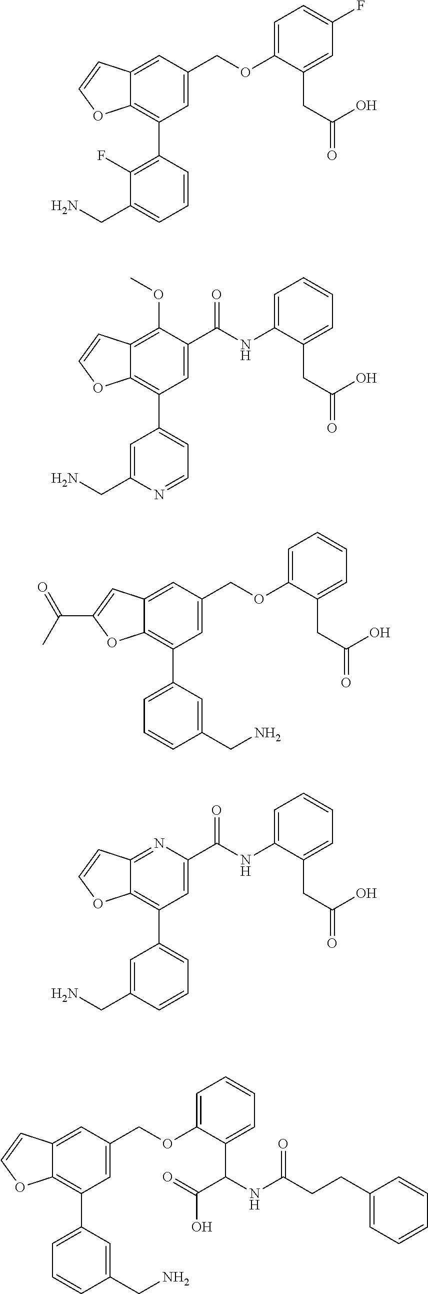 [SMILES: NCc1cccc(-c2cc(COc3ccc(F)cc3CC(=O)O)cc3ccoc23)c1F ; COc1c(C(=O)Nc2ccccc2CC(=O)O)cc(-c2ccnc(CN)c2)c2occc12 ; CC(=O)c1cc2cc(COc3ccccc3CC(=O)O)cc(-c3cccc(CN)c3)c2o1 ; NCc1cccc(-c2cc(C(=O)Nc3ccccc3CC(=O)O)nc3ccoc23)c1 ; NCc1cccc(-c2cc(COc3ccccc3C(NC(=O)CCc3ccccc3)C(=O)O)cc3ccoc23)c1]

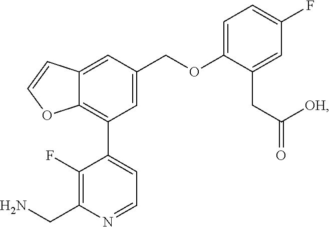 [SMILES: NCc1nccc(-c2cc(COc3ccc(F)cc3CC(=O)O)cc3ccoc23)c1F]

or a pharmaceutically acceptable salt or prodrug thereof; and a pharmaceutically acceptable carrier. In certain embodiments, the oral dosage form is a capsule.

Synthetic methods, characterization data, and assay data for the compounds listed above is disclosed in U.S. Provisional Patent Application No. 62/654,108, filed Apr. 6, 2018; International Patent Application No. PCT/US19/26054, filed Apr. 5, 2019; and U.S. patent application Ser. No. 16/511,642, filed Jul. 15, 2019; each of which is hereby incorporated by reference herein in its entirety.

### Pharmaceutical Compositions

The invention provides pharmaceutical compositions, each comprising one or more compounds of the invention, or pharmaceutically acceptable salts or prodrugs thereof, as described herein, and a pharmaceutically acceptable carrier. In certain embodiments, the pharmaceutical composition comprises a compound of the invention, or a pharmaceutically acceptable salt or prodrug thereof, and a pharmaceutically acceptable carrier. In certain embodiments, the pharmaceutical composition comprises a plurality of compounds of the invention, which may include pharmaceutically acceptable salts and/or prodrugs thereof, and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of a disease or condition characterized by aberrant complement system activity.

Pharmaceutical compositions of the invention can be prepared by combining one or more compounds of the invention with a pharmaceutically acceptable carrier and, optionally, one or more additional pharmaceutically active agents.

### Methods of Use

The present invention provides compounds, and pharmaceutically acceptable salts and prodrugs thereof, that are useful for treating or preventing a disease or condition characterized by aberrant complement system activity.

In certain aspects, the invention provides a compound of the invention, or a pharmaceutically acceptable salt or prodrug thereof, for use as a medicament.

In certain aspects, the invention provides methods of treating or preventing a disease or condition characterized by aberrant complement system activity. The method includes the step of administering to a subject in need thereof a therapeutically effective amount of a compound of the invention, or a pharmaceutically acceptable salt or prodrug thereof, thereby treating or preventing the disease or condition characterized by aberrant complement system activity. By reducing complement system activity in the subject, the disease or condition characterized by aberrant complement system activity is treated. In certain embodiments, administration is oral.

Alternatively, in certain aspects, the invention provides a compound of the invention, or a pharmaceutically acceptable salt or prodrug thereof, for treatment of a disease or condition characterized by aberrant complement system activity.

Alternatively, in certain aspects, the invention provides the use of a compound of the invention, or a pharmaceutically acceptable salt or prodrug thereof, for the manufacture of a medicament for use in treatment of a disease or condition characterized by aberrant complement system activity.

As used herein, a “disease or condition characterized by aberrant complement system activity” refers to any disease or condition in which it is desirable to reduce complement system activity. For example, it may be desirable to reduce complement system activity in the setting of inappropriate activation or hyperactivation of the complement system.

In certain embodiments, the disease or condition characterized by aberrant complement system activity is an immunological disorder.

In certain embodiments, the disease or condition characterized by aberrant complement system activity is a disease of the central nervous system.

In certain embodiments, the disease or condition characterized by aberrant complement system activity is a renal disease.

In certain embodiments, the disease or condition characterized by aberrant complement system activity is a cardiovascular disease.

In certain embodiments, the disease or condition characterized by aberrant complement system activity is a neurodegenerative disease or neurological disease.

In certain embodiments, the disease or condition characterized by aberrant complement system activity is selected from the group consisting of paroxysmal nocturnal hemoglobinuria (PNH), atypical hemolytic uremic syndrome, organ transplant rejection, myasthenia gravis, neuromyelitis optica, membranoproliferative glomerulonephritis, dense-deposit disease, cold agglutinin disease, and catastrophic antiphospholipid syndrome.

In certain embodiments, the disease or condition is paroxysmal nocturnal hemoglobinuria (PNH). In some such embodiments, the PNH is characterized by one or more of hemolytic anemia, thrombosis, and impaired bone marrow function. In certain embodiments, wherein the disease or condition is PNH, the orally administering suppresses hemolysis of erythrocytes or blocks C3 fragment deposition on erythrocytes or both. In some such embodiments, the suppression is greater than or equal to about 50%, such as about 80%, about 90%, or about 95%. In some such embodiments, the PNH is characterized by one or more of the following symptoms: fatigue, erectile dysfunction, headache, and abdominal pain. In certain embodiments, wherein the disease or condition is PNH, the orally administering prevents or reduces the occurrence of one or more or all of such symptoms. In some such embodiments, the reduction is greater than or equal to about 50%, such as about 80%, about 90%, or about 95%.

In certain embodiments, the disease or condition is atypical hemolytic uremic syndrome.

In certain embodiments, the disease or condition is organ transplant rejection.

In certain embodiments, the disease or condition is myasthenia gravis.

In certain embodiments, the disease or condition is neuromyelitis optica.

In certain embodiments, the disease or condition is membranoproliferative glomerulonephritis.

In certain embodiments, the disease or condition is dense-deposit disease.

In certain embodiments, the disease or condition is cold agglutinin disease.

In certain embodiments, the disease or condition is catastrophic antiphospholipid syndrome.

In other embodiments, the disease or condition characterized by aberrant complement system activity is adult respiratory distress syndrome, myocardial infarct, lung inflammation, hyperacute rejection (transplantation rejection), sepsis, cardiopulmonary bypass, burns, asthma, restenosis, multiple organ dysfunction syndrome, Guillain-Barré syndrome, hemorrhagic shock, paroxysmal nocturnal hemoglobinuria, glomerulonephritis, systemic lupus erythematosus, rheumatoid arthritis, infertility, Alzheimer's disease, organ rejection (transplantation), myasthenia gravis, multiple sclerosis, platelet storage, or hemodialysis.

In other embodiments, the disease or condition characterized by aberrant complement system activity is selected from the group consisting of antineutrophil cytoplasmic antibody (ANCA)-associated vasculitis (AAV), warm autoimmune hemolytic anemia, IgA nephropathy, C3 glomerulonephritis, and focal segmental glomerulosclerosis.

In certain embodiments, the disease or condition characterized by aberrant complement system activity is a hematological disorder.

In other embodiments, the disease or condition characterized by aberrant complement system activity is an ocular disorder or an eye disorder.

In certain embodiments, the disease or condition characterized by aberrant complement system activity is macular degeneration, age-related macular degeneration (AMD), macular edema, diabetic macular edema, choroidal neovascularization (CNV), uveitis, Behcet's uveitis, proliferative diabetic retinopathy, non-proliferative diabetic retinopathy, glaucoma, hypertensive retinopathy, a corneal neovascularization disease, post-corneal transplant rejection, a corneal dystrophic disease, an autoimmune dry eye disease, Stevens-Johnson syndrome, Sjogren's syndrome, an environmental dry eye disease, Fuchs' endothelial dystrophy, retinal vein occlusion, or post-operative inflammation.

In certain embodiments, the therapeutically effective amount is about 1 mg to about 6000 mg of the compound or a pharmaceutically acceptable salt or prodrug thereof, such as about 1 mg to about 3000 mg, such as about 1 mg to about 1500 mg, such as about 1 mg to about 1200 mg, about 1 mg to about 1000 mg, about 1 mg to about 800 mg, about 1 mg to about 600 mg, about 1 mg to about 400 mg, about 1 mg to about 300 mg, about 1 mg to about 200 mg, about 1 mg to about 100 mg, about 10 mg, about 30 mg, about 100 mg, about 200 mg, about 300 mg, about 400 mg, about 600 mg, about 800 mg, about 1000 mg, or about 1200 mg of the compound or a pharmaceutically acceptable salt thereof. In certain embodiments, the therapeutically effective amount is delivered per day.

In certain embodiments, the compound or a pharmaceutically acceptable salt or prodrug thereof is administered once daily. In other embodiments, the compound or a pharmaceutically acceptable salt or prodrug thereof is administered twice daily. In still other embodiments, the compound or a pharmaceutically acceptable salt or prodrug thereof is administered three times daily.

### Formulations, Routes of Administration, and Dosing

The compounds of the invention, and pharmaceutically acceptable salts or prodrugs thereof, as described herein, can be formulated as pharmaceutical compositions and administered to a mammalian host, such as a human patient, in a variety of forms adapted to the chosen route of administration, e.g., orally or parenterally, by intravenous, intraperitoneal, intramuscular, topical, or subcutaneous routes. Additional routes of administration are also contemplated by the invention. In certain embodiments, the present compounds are administered orally.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2% to about 60% of the weight of a given unit dosage form. The amount of active compound in such therapeutically useful compositions is such that an effective dosage level will be obtained.

The tablets, troches, pills, capsules, and the like may also contain the following diluents and carriers: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water or physiologically acceptable aqueous solution, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, methods of preparation can include vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the invention to the skin are known in the art; for example, see Jacquet et al. (U.S. Pat. No. 4,608,392; incorporated herein by reference), Geria (U.S. Pat. No. 4,992,478; incorporated herein by reference), Smith et al. (U.S. Pat. No. 4,559,157; incorporated herein by reference), and Wortzman (U.S. Pat. No. 4,820,508; incorporated herein by reference).

Useful dosages of the compounds of the invention can be determined, at least initially, by comparing their in vitro activity and in vivo activity in animal models. Methods for the extrapolation of effective dosages in mice, and other animals, to humans are known in the art; for example, see U.S. Pat. No. 4,938,949 (incorporated herein by reference).

The amount of the compound, or pharmaceutically acceptable salt or prodrug thereof, required for use in treatment will vary not only with the particular compound, salt, or prodrug selected but also with the route of administration, the nature of the condition being treated, and the age and condition of the patient and will be ultimately at the discretion of the attendant physician or clinician.

In general, however, a suitable dose will be in the range of from about 0.5 to about 100 mg/kg body weight of the recipient per day, e.g., from about 3 to about 90 mg/kg of body weight per day, from about 6 to about 75 mg per kilogram of body weight per day, from about of 10 to about 60 mg/kg of body weight per day, or from about 15 to about 50 mg/kg of body weight per day.

Compounds of the invention, or pharmaceutically acceptable salts or prodrugs thereof, can be conveniently formulated in unit dosage form; for example, containing 5 to 1000 mg, 10 to 750 mg, or 50 to 500 mg of active ingredient per unit dosage form. In one embodiment, the invention provides a composition comprising a compound of the invention, or pharmaceutically acceptable salts or prodrugs thereof, formulated in such a unit dosage form.

In certain aspects, the invention provides an oral dosage form comprising the compound, or a pharmaceutically acceptable salt or prodrug thereof, and a pharmaceutically acceptable carrier. In certain such embodiments, the oral dosage form comprises about 1 mg to about 1500 mg of the compound or a pharmaceutically acceptable salt or prodrug thereof, such as about 1 mg to about 1200 mg, about 1 mg to about 1000 mg, about 1 mg to about 800 mg, about 1 mg to about 600 mg, about 1 mg to about 400 mg, about 1 mg to about 300 mg, about 1 mg to about 200 mg, about 1 mg to about 100 mg, about 10 mg, about 30 mg, about 100 mg, about 200 mg, about 300 mg, about 400 mg, about 600 mg, about 800 mg, about 1000 mg, or about 1200 mg of the compound or a pharmaceutically acceptable salt or prodrug thereof.

In certain embodiments, the oral dosage form is a capsule. In other embodiments, the oral dosage form is a tablet. In some such embodiments, the tablet is a coated tablet.

The desired dose may conveniently be presented in a single dose or as divided doses to be administered at appropriate intervals, for example, as two, three, four or more sub-doses per day. The sub-dose itself may be further divided, e.g., into a number of discrete loosely spaced administrations. In certain embodiments, the desired dose is administered as a single dose per day. In certain embodiments, the desired dose is administered as divided doses, such as two sub-doses per day or three sub-doses per day.

Compounds of the invention, or pharmaceutically acceptable salts or prodrugs thereof, can be administered to patients for treatment for any period of time. Suitable duration of treatment may be based on several factors, such as the disease or condition being treated or other conditions the patient may have, and can be determined by those of ordinary skill in the art. In certain embodiments, the compound of the invention, or a pharmaceutically acceptable salt or prodrug thereof, can be administered for up to 48 weeks, such as 24 weeks, 12 weeks, 6 weeks, 3 weeks, 2 weeks, or 1 week. In certain embodiments, the compound of the invention, or a pharmaceutically acceptable salt or prodrug thereof, can be administered to a patient chronically.

Compounds of the invention, or pharmaceutically acceptable salts or prodrugs thereof, can be administered to patients in either the fasted or the fed state (e.g., with or after a high-fat meal).

Compounds of the invention, or pharmaceutically acceptable salts or prodrugs thereof, can also be administered in combination with other therapeutic agents, for example, other agents that are useful for treating or preventing a disease or condition characterized by aberrant complement system activity or a disease or condition disclosed herein. In certain embodiments, compounds of the invention, and pharmaceutically acceptable salts or prodrugs thereof, can also be administered in combination with one or more other therapeutic agents that are useful for treating or preventing an ocular disorder or eye disorder. In certain embodiments, the additional therapeutic agent is at least one of a Factor B inhibitor, a C3 convertase inhibitor, a C3a inhibitor, a C5 convertase inhibitor, a plasma kallikrein inhibitor and an angiogenesis inhibitor. In certain embodiments, the additional therapeutic agent is at least one of TT-30, AMY-101, ADL-2, ACH-4471, LNP-023, eculizumab, ravulizumab, ALXN1210, SKY-59, ABP-959, REGN-959, RA-101495, conversin, ALNCCS, a VEGF antagonist, BCX4161, BCX7353, KDV001, KDV818, KDV824, KDV900, ecallantide, DX-2930, lanadelumab, a compound described in PCT/US2001/032582, a compound described in PCT/US2015/019535, a compound described in PCT/US2016/054619, and a compound described in PCT/US2017/058685.

Other delivery systems can include time-release, delayed release, or sustained release delivery systems such as are well-known in the art. Such systems can avoid repeated administrations of the active compound, increasing convenience to the subject and the physician. Many types of release delivery systems are available and known to those of ordinary skill in the art. Use of a long-term sustained release implant may be desirable. Long-term release, as used herein, means that the delivery system or is implant constructed and arranged to deliver therapeutic levels of the active ingredient for at least 30 days, and preferably 60 days.

In certain embodiments, a compound of the invention is formulated for intraocular administration, for example direct injection or insertion within or in association with an intraocular medical device. In certain embodiments, a compound of the invention is formulated as an ophthalmic solution. In certain embodiments, a compound of the invention can be administered via ocular delivery, for example, by local ocular administration, including topical, intravitreal, periocular, transscleral, retrobulbar, juxtascleral, suprachoroidal, or sub-tenon administration. A compound of the invention can be administered via ocular delivery either alone or in combination with one or more additional therapeutic agents.

The compounds of the invention may be formulated for depositing into a medical device, which may include any of a variety of conventional grafts, stents, including stent grafts, catheters, balloons, baskets, or other device that can be deployed or permanently implanted within a body lumen. As a particular example, it would be desirable to have devices and methods which can deliver compounds of the invention to the region of a body which has been treated by interventional technique.

In exemplary embodiment, a compound of the invention may be deposited within a medical device, such as a stent, and delivered to the treatment site for treatment of a portion of the body.

Stents have been used as delivery vehicles for therapeutic agents (i.e., drugs). Intravascular stents are generally permanently implanted in coronary or peripheral vessels. Stent designs include those of U.S. Pat. No. 4,733,655 (Palmaz), U.S. Pat. No. 4,800,882 (Gianturco), or U.S. Pat. No. 4,886,062 (Wiktor). Such designs include both metal and polymeric stents, as well as self-expanding and balloon-expandable stents. Stents may also be used to deliver a drug at the site of contact with the vasculature, as disclosed in U.S. Pat. No. 5,102,417 (Palmaz), U.S. Pat. No. 5,419,760 (Narciso, Jr.), U.S. Pat. No. 5,429,634 (Narciso, Jr.), and in International Patent Application Nos. WO 91/12779 (Medtronic, Inc.) and WO 90/13332 (Cedars-Sanai Medical Center), for example.

The term “deposited” means that the compound is coated, adsorbed, placed, or otherwise incorporated into the device by methods known in the art. For example, the compound may be embedded and released from within (“matrix type”) or surrounded by and released through (“reservoir type”) polymer materials that coat or span the medical device. In the latter example, the compound may be entrapped within the polymer materials or coupled to the polymer materials using one or more the techniques for generating such materials known in the art. In other formulations, the compound may be linked to the surface of the medical device without the need for a coating, for example by means of detachable bonds, and release with time or can be removed by active mechanical or chemical processes. In other formulations, the compound may be in a permanently immobilized form that presents the compound at the implantation site.

In certain embodiments, the compound may be incorporated with polymer compositions during the formation of biocompatible coatings for medical devices, such as stents. The coatings produced from these components are typically homogeneous and are useful for coating a number of devices designed for implantation.

The polymer may be either a biostable or a bioabsorbable polymer depending on the desired rate of release or the desired degree of polymer stability, but frequently a bioabsorbable polymer is preferred for this embodiment since, unlike a biostable polymer, it will not be present long after implantation to cause any adverse, chronic local response. Bioabsorbable polymers that could be used include, but are not limited to, poly(L-lactic acid), polycaprolactone, polyglycolide (PGA), poly(lactide-co-glycolide) (PLLA/PGA), poly(hydroxybutyrate), poly(hydroxybutyrate-co-valerate), polydioxanone, polyorthoester, polyanhydride, poly(glycolic acid), poly(D-lactic acid), poly(L-lactic acid), poly(D,L-lactic acid), poly(D, L-lactide) (PLA), poly (L-lactide) (PLLA), poly(glycolic acid-co-trimethylene carbonate) (PGA/PTMC), polyethylene oxide (PEO), polydioxanone (PDS), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid, polyepsilon caprolactone, polyhydroxy butyric acid, polyorthoesters, polyacetals, polydihydropyrans, polycyanoacrylates, cross linked or amphipathic block copolymers of hydrogels, and other suitable bioabsorbable poplymers known in the art. Also, biostable polymers with a relatively low chronic tissue response such as polyurethanes, silicones, and polyesters could be used, and other polymers could also be used if they can be dissolved and cured or polymerized on the medical device such as polyolefins, polyisobutylene and ethylene-alphaolefin copolymers; acrylic polymers and copolymers, vinyl halide polymers and copolymers, such as polyvinyl chloride; polyvinylpyrrolidone; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; pyran copolymer; polyhydroxy-propyl-methacrylamide-phenol; polyhydroxyethyl-aspartamide-phenol; polyethyleneoxide-polylysine substituted with palmitoyl residues; polyamides, such as Nylon 66 and polycaprolactam; alkyd resins, polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

Polymers and semipermeable polymer matrices may be formed into shaped articles, such as valves, stents, tubing, prostheses and the like.

In certain embodiments of the invention, the compound of the invention is coupled to a polymer or semipermeable polymer matrix that is formed as a stent or stent-graft device.

Typically, polymers are applied to the surface of an implantable device by spin coating, dipping, or spraying. Additional methods known in the art can also be utilized for this purpose. Methods of spraying include traditional methods as well as microdeposition techniques with an inkjet type of dispenser. Additionally, a polymer can be deposited on an implantable device using photo-patterning to place the polymer on only specific portions of the device. This coating of the device provides a uniform layer around the device which allows for improved diffusion of various analytes through the device coating.

In certain embodiments of the invention, the compound is formulated for release from the polymer coating into the environment in which the medical device is placed. Preferably, the compound is released in a controlled manner over an extended time frame (e.g., months) using at least one of several well-known techniques involving polymer carriers or layers to control elution. Some of these techniques are described in U.S. Patent Application 2004/0243225A1, the entire disclosure of which is incorporated herein in its entirety.

Moreover, as described for example in U.S. Pat. No. 6,770,729, which is incorporated herein in its entirety, the reagents and reaction conditions of the polymer compositions can be manipulated so that the release of the compound from the polymer coating can be controlled. For example, the diffusion coefficient of the one or more polymer coatings can be modulated to control the release of the compound from the polymer coating. In a variation on this theme, the diffusion coefficient of the one or more polymer coatings can be controlled to modulate the ability of an analyte that is present in the environment in which the medical device is placed (e.g., an analyte that facilitates the breakdown or hydrolysis of some portion of the polymer) to access one or more components within the polymer composition (and for example, thereby modulate the release of the compound from the polymer coating). Yet another embodiment of the invention includes a device having a plurality of polymer coatings, each having a plurality of diffusion coefficients. In such embodiments of the invention, the release of the compound from the polymer coating can be modulated by the plurality of polymer coatings.

In yet another embodiment of the invention, the release of the compound from the polymer coating is controlled by modulating one or more of the properties of the polymer composition, such as the presence of one or more endogenous or exogenous compounds, or alternatively, the pH of the polymer composition. For example, certain polymer compositions can be designed to release a compound in response to a decrease in the pH of the polymer composition.

### Example

Introduction:

Paroxysmal nocturnal hemoglobinuria (PNH) is a rare, life-threatening disease characterized by hemolytic anemia, thrombosis, and impaired bone marrow function. Uncontrolled activity of the alternative pathway (AP) of complement leads to intravascular hemolysis triggered by the membrane attack complex (C5b-9) formation in patients with PNH. Terminal complement inhibition by IV administration of eculizumab or ravulizumab reduces intravascular hemolysis and is the standard of care for PNH. However, up to one-third of patients treated with eculizumab continue to be transfusion dependent as a result of on-going AP activation and C3-mediated extravascular hemolysis (Kelly R J, et al. Blood 2011; 117(25):6786-6792). The compounds of the invention are novel, potent, selective, and orally bioavailable small-molecule inhibitors of human complement factor D. In preclinical in vitro studies, compounds of the invention suppressed the AP-mediated hemolysis of erythrocytes from patients with PNH and blocked AP-mediated C3 fragment deposition on PNH erythrocytes at low nanomolar drug concentrations. Oral dosing of the compounds achieved sustained >95% suppression of AP-mediated hemolysis in preclinical in vivo studies of complement activity in monkeys. Therefore, targeting proximal complement inhibition by the compounds of the invention will potentially prevent intravascular and avoid extravascular hemolysis associated with opsonization of PNH erythrocytes by C3 in patients with PNH. A first-in-human phase 1 study of compounds of the invention in healthy subjects is underway.

### Methods:

An ongoing randomized, placebo-controlled, phase 1 study is investigating the safety, tolerability, pharmacokinetics (PK), and pharmacodynamics (PD) of single (Part 1) and multiple (Part 2) ascending oral doses of a compound disclosed herein (“Compound”) in healthy subjects, and of multiple ascending oral doses of the Compound in subjects with PNH (Part 3). The mean age of all subjects in Parts 1 and 2 was 34.3 years and 50.9% of the subjects were male. Parts 1 and 2 of the study are participant and investigator masked (double blind); Part 3 of the study is not masked.

Parts 1 and 2:

Healthy male and female subjects were recruited and randomized into cohorts in two Parts.

### Part 1—Single Ascending Dose Evaluation

Cohort 1, Regimen A: 10 mg Compound or placebo orally, ×1 dose
Cohort 2, Regimen B: 30 mg Compound or placebo orally, ×1 dose
Cohort 3, Regimen C: 100 mg Compound or placebo orally, ×1 dose
Cohort 4, Regimen D, Period 1: 300 mg Compound or placebo orally, ×1 dose (fasted)
Cohort 4, Regimen D, Period 2: 300 mg Compound or placebo orally, ×1 dose (administration after a high fat meal); dosing in Period 2 was separated from dosing in Period 1 by 7 days
Cohort 5, Regimen E: 600 mg Compound or placebo orally, ×1 dose
Cohort 6, Regimen F: 1200 mg Compound or placebo, ×1 dose

Subjects in Part 1 were treated with a single oral dose of study drug (Compound or placebo) per dose cohort. Escalation to the next higher dose level in Part 1 occurred only after satisfactory review of clinical safety and pharmacokinetic data. Subjects in Part 1 Cohort 4 initially received a single dose under fasting conditions; after an adequate washout period, all subjects in Part 1 Cohort 4 were given a second dose after a high-fat meal. Subjects received the same study drug and dose (Compound or placebo) in both dosing periods.

### Part 2—Multiple Ascending Dose Evaluation

Cohort 1, Regimen G: 100 mg Compound or placebo/day orally, 7 days (13 doses)
Cohort 2, Regimen H: 200 mg Compound or placebo/day orally, 7 days (13 doses)
Cohort 3, Regimen I: 100 mg Compound or placebo/day orally, 14 days (27 doses)
Cohort 4, Regimen J: 400 mg Compound or placebo/day orally, 3 days (5 doses)
Cohort 5, Regimen K: 800 mg Compound or placebo/day orally, 3 days (5 doses)

Subjects in Part 2 were treated with either a 3-, 7- or 14-day course of study drug (Compound or placebo) administered orally. For all cohorts, the daily dose was split into 2 equal doses, administered 12 hours apart (i.e., BID; Q12 hr). Subjects received a final morning dose of study drug (Compound or placebo) on the last day of dosing. Escalation to the next higher dose level in Part 2 occurred only after satisfactory review of clinical safety and pharmacokinetic data. Subjects in Part 2 were administered a prophylactic antibiotic or vaccination against Neisseria meningitides (Cohort 3) when Compound was administered.

Safety and tolerability in Parts 1 and 2 were evaluated via clinical and laboratory monitoring. Plasma concentrations of the Compound in Parts 1 and 2 were measured in serial post-dose samples using a validated LC/MS/MS assay. PD effects of the Compound in Parts 1 and 2 was assessed using multiple assays, such as, but not limited to, inhibition of serum complement AP activity measured by AP specific Wieslab assay, lysis of rabbit red blood cells by human serum (AP hemolysis assay), and the change in plasma AP pathway biomarker Bb (the split product of complement factor B by the enzymatic action of factor D).

The primary endpoints of Parts 1 and 2 were safety and tolerability of the Compound based on laboratory and examination findings. Secondary endpoints included pharmacokinetic parameters [such as, but not limited to, the maximum (peak) plasma drug concentration observed (Cₘₐₓ), median time to reach maximum (peak) plasma drug concentration following drug administration (Tₘₐₓ), the area under the plasma concentration-time curve from time 0 through 24 hours (AUC₀₋₂₄), AUC extrapolated to infinite time (AUC_{inf}), terminal elimination half-life (t_{1/2}), and dose proportionality] and the pharmacodynamic parameters in the paragraph above.

Results:

Part 1—Single Ascending Dose Evaluation

Forty-eight healthy subjects completed dosing in the 6 single ascending dose cohorts (10 mg, 30 mg, 100 mg, 300 mg, 600 mg, and 1200 mg) in Part 1. For all Cohorts of Part 1, 8 subjects were enrolled per Cohort (6 Compound and 2 placebo treated subjects per cohort). Subjects in Cohorts 1-3 were orally administered Compound in a single dose administered as a single capsule containing 10 mg, 30 mg, or 100 mg of Compound, respectively. Subjects in Cohort 4 were orally administered 300 mg Compound in a single dose administered as two 150 mg capsules. Subjects in Cohort 5 were orally administered 600 mg Compound in a single dose administered as three 200 mg capsules. Subjects in Cohort 6 were orally administered 1200 mg Compound in a single dose administered as six 200 mg capsules. Placebo for each Cohort was provided in matching form.

For Cohort 4, Period 1, subjects initially received a first single dose of study drug (Compound or placebo) as described above under fasting conditions (fasted). For Cohort 4, Period 2, after a washout period of 7 days all subjects were given a second single dose of study drug (Compound or placebo) as described above after a high-fat meal (fed). Subjects received the same study drug and dose (Compound or placebo) in both Periods 1 and 2.

Table 1 provides a summary of key pharmacokinetic parameters of Compound in healthy subjects following oral administration of escalating doses of Compound as described above for Part 1.

TABLE 1
Geometric mean (CV %) key pharmacokinetic parameters of Compound in healthy subjects
following oral administration of escalating doses of Compound in capsules.

| | Tₘₐₓ^{b} | | Cₘₐₓ | | AUC₍₀₋₂₄₎ | | AUC_{(0-inf)} | | t_{1/2}^{c} | |
|---|---|---|---|---|---|---|---|---|---|---|
| Regimen^{a} | (h) | | (ng/mL) | | (ng*h/mL) | | (ng*h/mL) | | (h) | |
| Cohort 1 | 1 | (0.5-2) | 79.8 | (42.2%) | 336 | (14.6%) | 357 | (53.7%) | 6.47 | (23.7%) |
| (10 mg | | | | | | | | | | |
| Compound) | | | | | | | | | | |
| Cohort 2 | 2 | (1-2) | 232 | (43.4%) | 968 | (41.5%) | 999 | (41.3%) | 4.51 | (10.2%) |
| (30 mg | | | | | | | | | | |
| Compound) | | | | | | | | | | |
| Cohort 3 | 1 | (0.5-2) | 869 | (42.9%) | 3700 | (30.4%) | 3990 | (29.3%) | 10.5^{d} | (19.4%) |
| (100 mg | | | | | | | | | | |
| Compound) | | | | | | | | | | |
| Cohort 4, | 1.5 | (0.5-3) | 1990 | (49.7%) | 9540 | (31.5%) | 9780 | (31.2%) | 4.3 | (15.4%) |
| Period 1 | | | | | | | | | | |
| (300 mg | | | | | | | | | | |
| Compound, | | | | | | | | | | |
| fasted) | | | | | | | | | | |
| Cohort 4, | 3.5 | (2-5) | 2360 | (26.8%) | 12700 | (22.2%) | 13000 | (22.2%) | 4.2 | (12.8%) |
| Period 2 | | | | | | | | | | |
| (300 mg | | | | | | | | | | |
| Compound, | | | | | | | | | | |
| fed) | | | | | | | | | | |
| Cohort 5 | 1.5 | (0.5-5) | 5600 | (22.1%) | 23400 | (15.4%) | 24300 | (15.3%) | 6.8 | (24.3%) |
| (600 mg | | | | | | | | | | |
| Compound) | | | | | | | | | | |
| Cohort 6 | 2.5 | (2-5) | 10350 | (43.9%) | 50150 | (35.6%) | 52010 | (35.3%) | 5.7 | (31.2%) |
| (1200 mg | | | | | | | | | | |
| Compound) | | | | | | | | | | |

^{a}All doses were administered in the fasted state unless otherwise specified.
^{b}Median and range.
^{c}Calculated based on PK samples from 0-24 h.
^{d}Calculated based on PK samples from 0-72 hours for most of Cohort 3 and 24 hours for straggler subjects.

Compound administered as ascending oral doses of 10 mg to 1200 mg was safe and generally well tolerated in all subjects in all Cohorts. There were no study discontinuations, no serious adverse events, and no clinically significant changes is safety laboratory parameters. Headache was the most frequently reported treatment-emergent adverse event; incidence of headache had no dose response relationship. All headaches were mild in severity and resolved spontaneously.

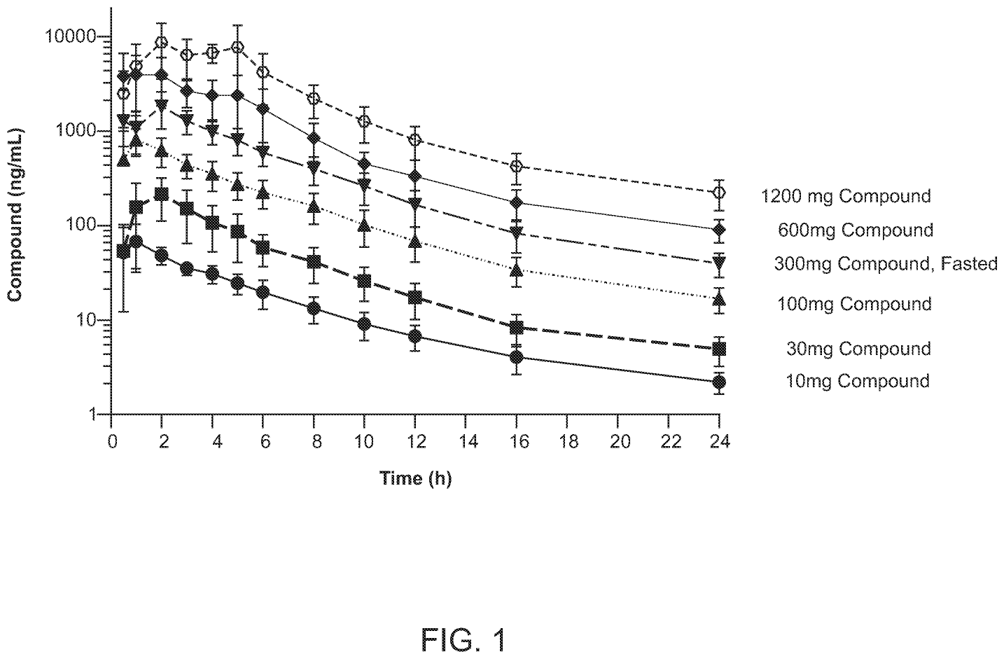

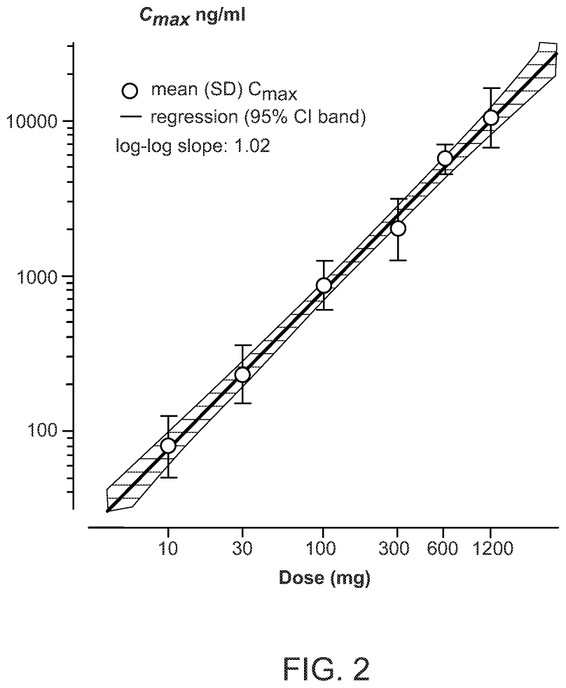

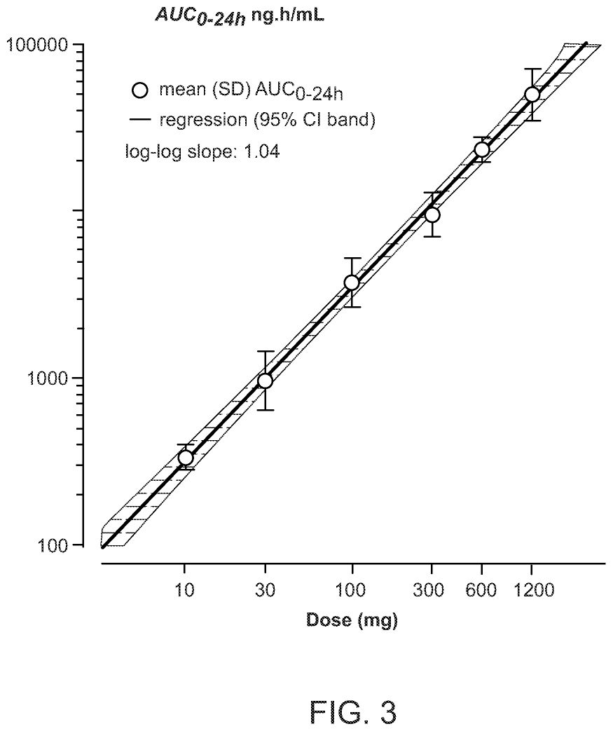

PK parameters of ascending oral doses of Compound for all Cohorts in Part 1 were well characterized over the 24-hour sampling period (FIG. 1; Table 1) and approximately linear and dose proportional (FIGS. 2 and 3). Mean Compound concentration at 12 hours post dose was within or above the anticipated threshold of 8 to 10 times the factor D EC₅₀ for single ascending doses ≥300 mg.

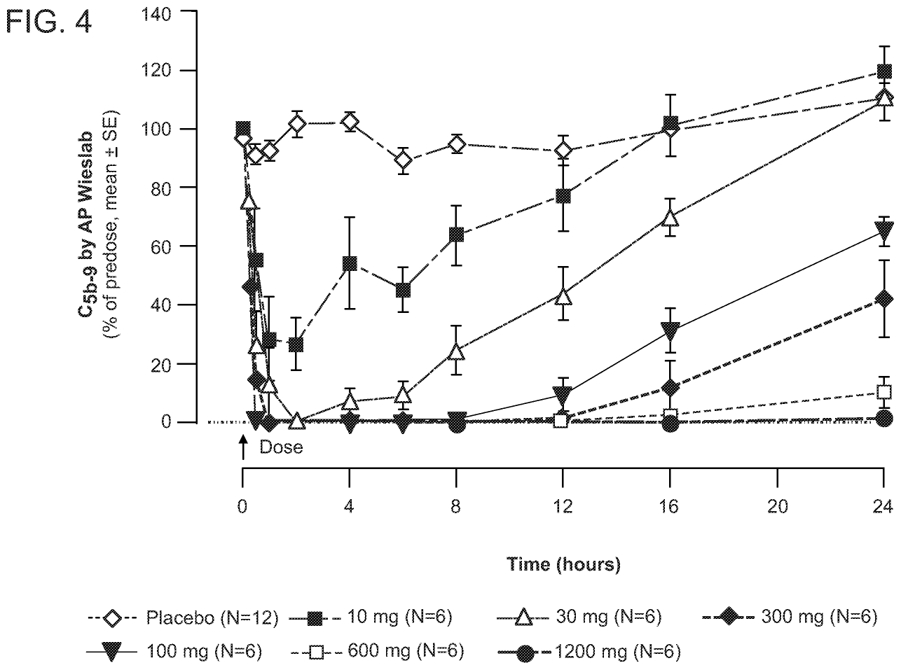

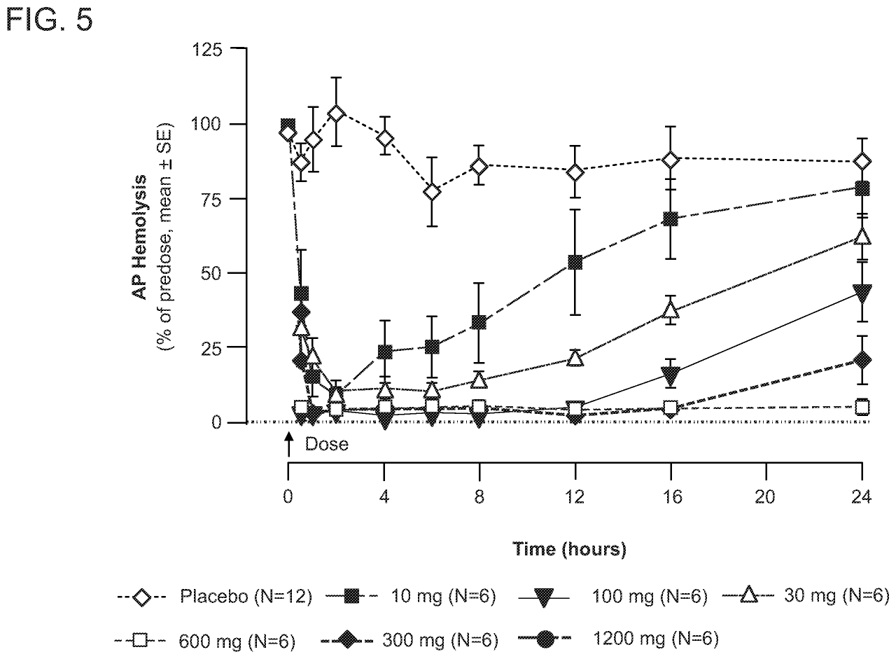

Dose-dependent suppression of the AP complement functional activity was observed in Part 1. Dose-dependent inhibition of AP Wieslab activity was detected, with greater than 90% suppression of AP Wieslab activity sustained for 12 hours post-dose in Cohort 3 (90.4%±13.8% inhibition) and Cohort 4 (97.8%±4.0% inhibition; data from Period 1) (FIG. 4). Approximately 90% suppression of AP Wieslab activity was sustained for over 24 hours post-dose in Cohort 5 (89.7%±12.9% inhibition) and Cohort 6 (98.5%±2.7% inhibition), with near complete suppression observed for 24 hours post-dose in Cohort 6 (FIG. 4). Dose-dependent inhibition of AP hemolysis was also detected with greater than 90% suppression of AP hemolysis sustained for 12 hours post-dose in Cohort 3 (95.0%±3.2% inhibition) and Cohort 4 (97.3%±2.5% inhibition; data from Period 1) (FIG. 5). Approximately 95% suppression of AP hemolysis was sustained for over 24 hours post-dose in Cohort 5 (94.5%±4.2% inhibition) and Cohort 6 (95.2%±5.3% inhibition) (FIG. 5). FIGS. 4 and 5 shows ≥95% suppression of AP activity was observed for single ascending doses ≥300 mg for 12 hours following administration, with the duration of suppression increasing with dose. Oral administration of Compound suppressed Factor Bb formation by over 50% in all Cohorts (data not shown). The inhibition values shown above are compared to pre-dose values and are mean values (±SD of the mean) derived from all subjects in each Cohort.

In comparing the exposure of Compound administered after food to that of fasted administration, the median Tₘₐₓ was slightly delayed, and Cₘₐₓ and AUC were slightly higher, yielding a modest increase in exposure following administration with a high-fat meal (Table 1). In light of the minimal impact observed on PK when comparing the fed versus fasted data, dosing for the fifth and sixth cohorts in Part 1 was continued in the fasting state, and Parts 2 and 3 proceeded in the fasted state.

In summary, for Part 1 Compound administered as a single oral dose of 10 mg to 1200 mg was safe and generally well tolerated in all subjects with no Compound-related safety concerns of note. Furthermore, Compound administered as a single oral dose of 100 mg to 1200 mg demonstrated dose-dependent suppression of the AP functional activity.

Part 2—Multiple Ascending Dose Evaluation

Part 2 is currently ongoing. Additional Cohorts in Part 2 may be enrolled to test higher supratherapeutic dosing and/or 1 time per day (i.e., QD) administration in healthy subjects. 55 healthy subjects completed dosing in the 5 multiple ascending dose cohorts (100 mg and 200 mg per day for 7 days, 400 mg and 800 mg per day for 3 days, or 100 mg per day for 14 days, all fasting state) in Part 2. For all cohorts, the daily dose was split into 2 equal doses, administered 12 hours apart (i.e., BID; Q12 hr) and Compound and placebo were administered in capsule dosage form. For Cohort 1, subjects received oral administration of Compound (N=10) or matching placebo (N=2), 50 mg BID for 6 days with a final dose of 50 mg on the morning of day 7. For Cohort 2, subjects received oral administration of Compound (N=10) or matching placebo (N=2), 100 mg BID for 6 days with a final dose of 100 mg on the morning of day 7. For Cohort 3, subjects received oral administration of Compound (N=10) or matching placebo (N=2), 50 mg BID for 13 days with a final dose of 50 mg on the morning of day 14. For Cohort 4, subjects received oral administration of Compound (N=10) or matching placebo (N=2), 200 mg BID for 2 days with a final dose of 200 mg on the morning of day 3. For Cohort 5, subjects received oral administration of Compound (N=10) or matching placebo (N=2), 400 mg BID for 2 days with a final dose of 400 mg on the morning of day 3. All subjects completed their respective dosing regimens except for Cohort 3, where 7 of the 12 subjects completed the dosing regimen. Those subjects that discontinued early and were randomized to receive Compound were excluded from the Day 14 PK analysis.

All enrolled subjects in each Cohort had PK samples collected through at least 48 hours after dosing on Day 1 and all enrolled subject that completed their dosing regimen had PK samples collected through at least 24 hours after dosing on the final day of dosing. After dosing on Day 1, plasma concentrations of Compound were quantifiable through 12 hours in all subjects in all Cohorts and after administration of the final dose, plasma concentrations of Compound were quantifiable through at least 24 hours in all subjects in all Cohorts. Plasma concentrations of Compound were quantifiable before administration of the final dose in all subject in all Cohorts. For Day 1 PK data, only data through 12 hours is reported, as all data after the second dose at 12 hours is reflective of accumulation.

Table 2 provides a summary of key pharmacokinetic parameters of Compound in healthy subjects following oral administration of Compound every 12 hours as described above for Part 2.

Compound administered as multiple ascending oral doses for 3, 7, or 14 days in Part 2 was safe and generally well tolerated in all subjects in all Cohorts (with the exception of Cohort 3) with no serious adverse events, no clinically significant changes is safety laboratory parameters and no dose-dependent safety signals were observed. There were 5 study discontinuations (all in Cohort 3). Benign rash in a number of subjects was observed and was self-limited and resolved within a median of 5 days of onset. Incidence of skin rashes had no dose response relationship.

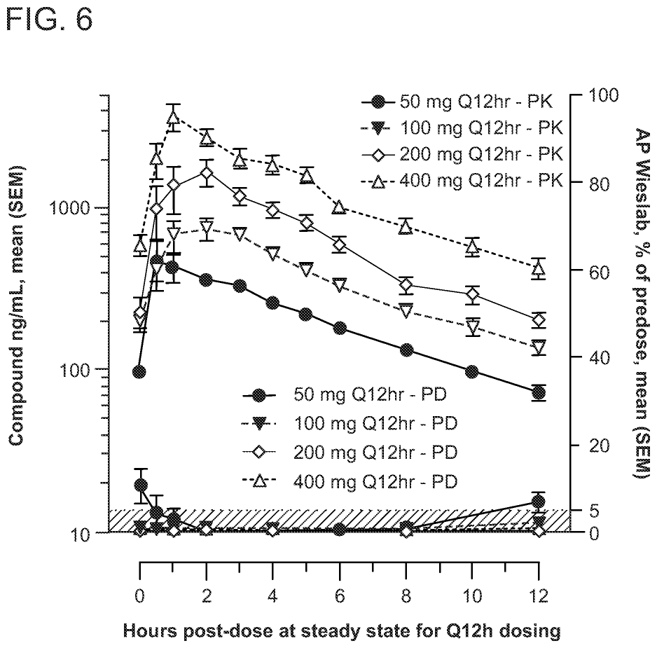

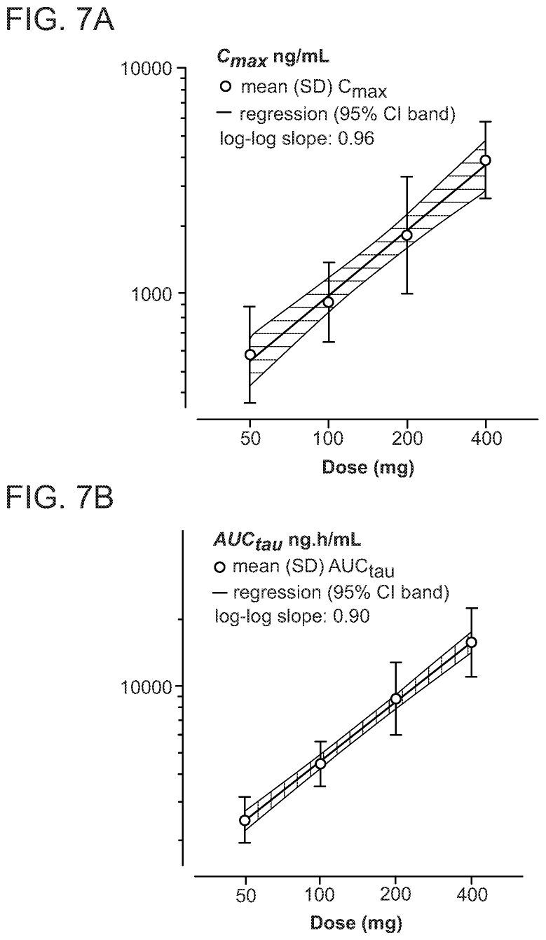

Compound exposure for these multiple ascending doses was well characterized over the sampling period (FIG. 6; Table 2) and approximately linear and dose-proportional (FIGS. 7A-B). Mean Compound concentration at 12 hours post dose was within or above the anticipated threshold of 8 to 10 times the factor D EC₅₀ for multiple ascending doses ≥200 mg/day. Compound exposure was approximately 50% higher in Cohort 3 as compared to Cohort 1 both on Day 1 and at steady state, despite the same dose being administered in each Cohort. Following an investigation no bias or process error was identified and the increase in exposure was attributed to be due to inherent pharmacokinetic variability and small sample size. Increase in exposure to Compound was mild in all Cohorts (see accumulation ratios Day 7/Day 1 in Table 2). Based on data for these five cohorts, Tₘₐₓ does not appear to be impacted by multiple dosing. Of note, the accumulation ratio in Cohorts 4 and 5 was highly consistent with those of Cohorts 1 through 3, suggesting that steady state is reached following 5 doses of Compound every 12 hours (Table 2).

TABLE 2
Cumulative geometric mean (CV %) of key pharmacokinetic parameters of Compound in healthy
subjects following oral administration of Compound in capsules every 12 hours.

| | Cohort 1, | | Cohort 2, | | Cohort 3, | | Cohort 4, | | Cohort 5, | |
|---|---|---|---|---|---|---|---|---|---|---|
| | 1^{st} Dose | | 1^{st} Dose | | 1^{st} Dose | | 1^{st} Dose | | 1^{st} Dose | |
| Regimen | 50 mg BID | | 100 mg BID | | 50 mg BID | | 200 mg BID | | 400 mg BID | |
| Tₘₐₓ^{a} | 2 | (0.5-3) | 2 | (0.5-3) | 1 | (0.5-2) | 2 | (1-6) | 1.5 | (0.4-4) |
| Cₘₐₓ | 405 | (44.9) | 901 | (32.1) | 602 | (30.7) | 1243 | (45.9) | 3137 | (41.5) |
| (ng/mL) | | | | | | | | | | |
| C₁₂^{b} | 39.4 | (44.2) | 67.8 | (31.1) | 49.0 | (36.9) | 125.8 | (47.2) | 223.2 | (36.4) |
| (ng/mL) | | | | | | | | | | |
| AUC₀₋₁₂ | 1662 | (42.3) | 3154 | (19.8) | 2180 | (30.9) | 5873 | (38.1) | 11479 | (23.6) |
| (ng*h/mL) | | | | | | | | | | |
| AUC₀₋₂₄ | 3141 | (33.4) | 6151 | (18.5) | 4095 | (30.4) | 11952 | (32.8) | 25193 | (31.2) |
| (ng*h/mL) | | | | | | | | | | |

| | Cohort 1, | | Cohort 2, | | Cohort 3, | | Cohort 4, | | Cohort 5, | |
|---|---|---|---|---|---|---|---|---|---|---|
| | Day 7 | | Day 7 | | Day 14 | | Day 3 | | Day 3 | |
| Regimen | 50 mg BID | | 100 mg BID | | 50 mg BID | | 200 mg BID | | 400 mg BID | |
| Tₘₐₓ^{a} | 1.5 | (0.5-3) | 2.5 | (1-3) | 1 | (0.5-3) | 2 | (0.5-5) | 1 | (0.5-5) |
| Cₘₐₓ | 539 | (52.0) | 918 | (41.9) | 883 | (19.6) | 1817 | (65.4) | 3958 | (42.0) |
| (ng/mL) | | | | | | | | | | |
| C₁₂^{b} | 73.2 | (34.4) | 136.4 | (30.8) | 84.3 | (28.1) | 222.1 | (31.4) | 431.1 | (44.0) |
| (ng/mL) | | | | | | | | | | |
| T_{1/2}^{c} | 6.5 | (29.8) | 5.8 | (22.3) | 5.8 | (9.6) | 6.2 | (14.1) | 6.9 | (23.7) |
| AUCₜₐᵤ | 2485 | (24.8) | 4460 | (23.9) | 3818 | (11.1) | 8809 | (39.2) | 15918 | (37.1) |
| (ng*h/mL) | | | | | | | | | | |
| AUC_{0-inf} | 3449 | (27.4) | 5968 | (29.8) | 4863 | (13.2) | 11320 | (35.9) | 20394 | (40.9) |
| (ng*h/mL) | | | | | | | | | | |
| Accumulation | 1.50 | (34.2) | 1.41 | (20.5) | 1.48 | (21.6) | 1.50 | (18.8) | 1.39 | (18.6) |
| Ratio^{d} | | | | | | | | | | |

^{a}Median and range.
^{b}Arithmetic mean and CV.
^{c}Calculated for Cohorts 1 and 2 using best-fit slope of all available PK time points and for Cohorts 3-5 using elimination rate constant estimated via slope of Compound concentration vs time profiles from 8 to 24 hours.
^{d}Calculated as ratio of steady-state AUCtau to Day 1 AUC₀₋₁₂

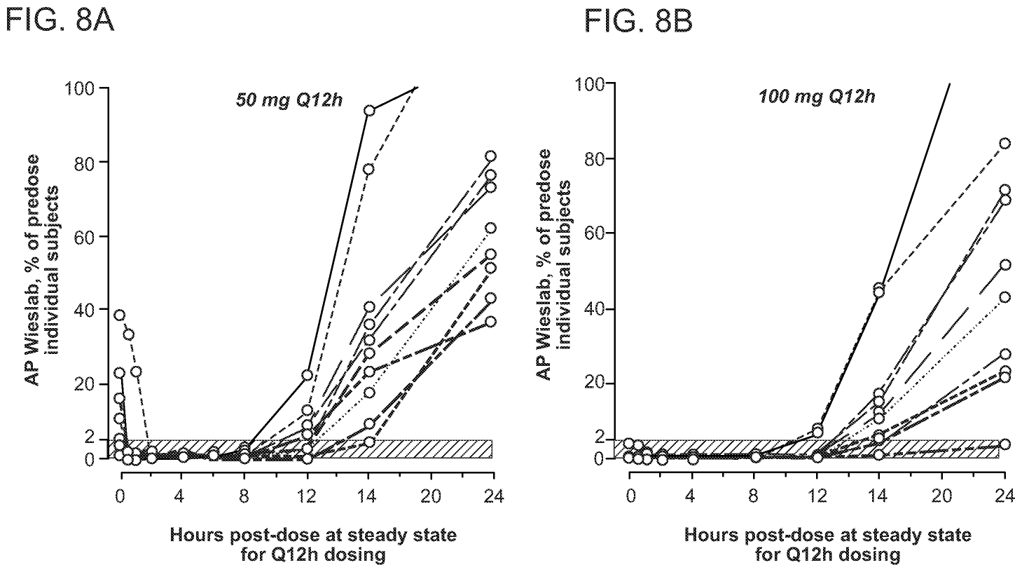

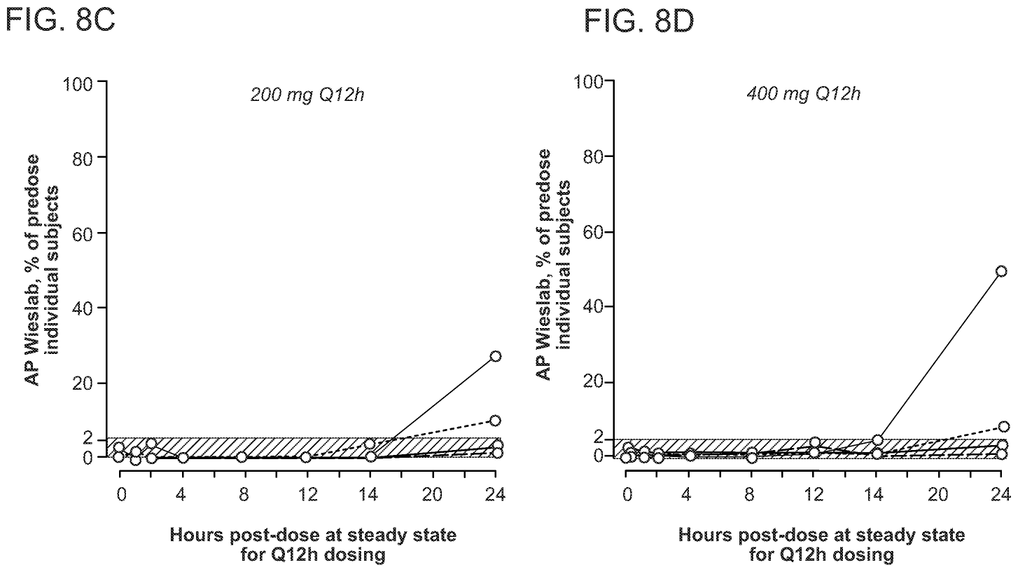

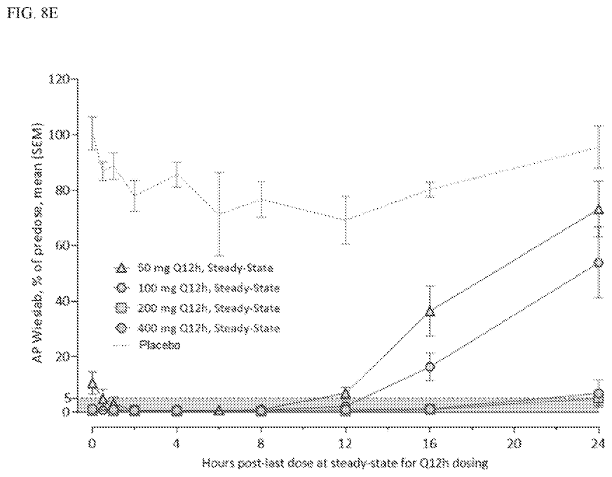

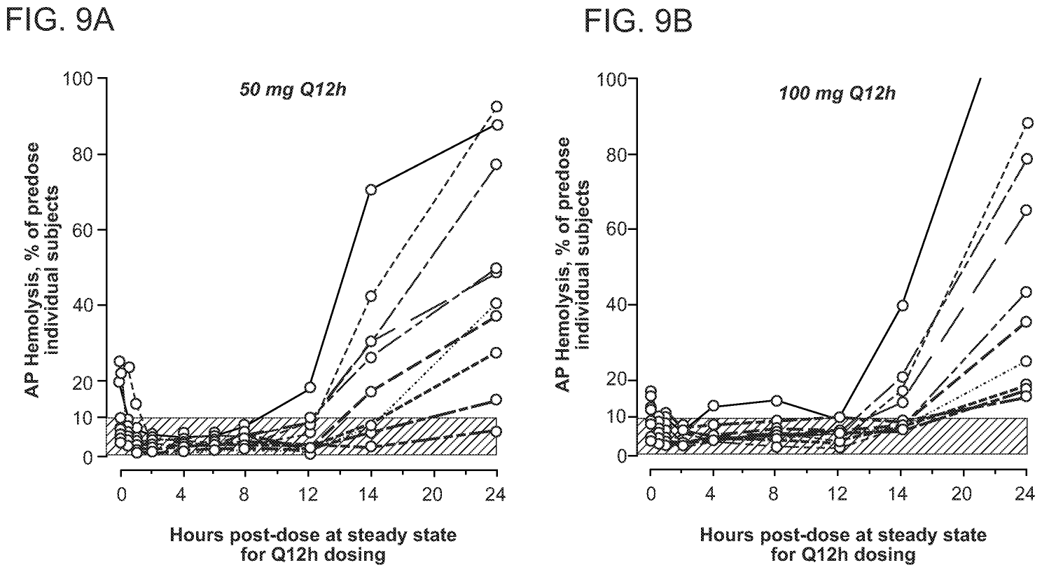

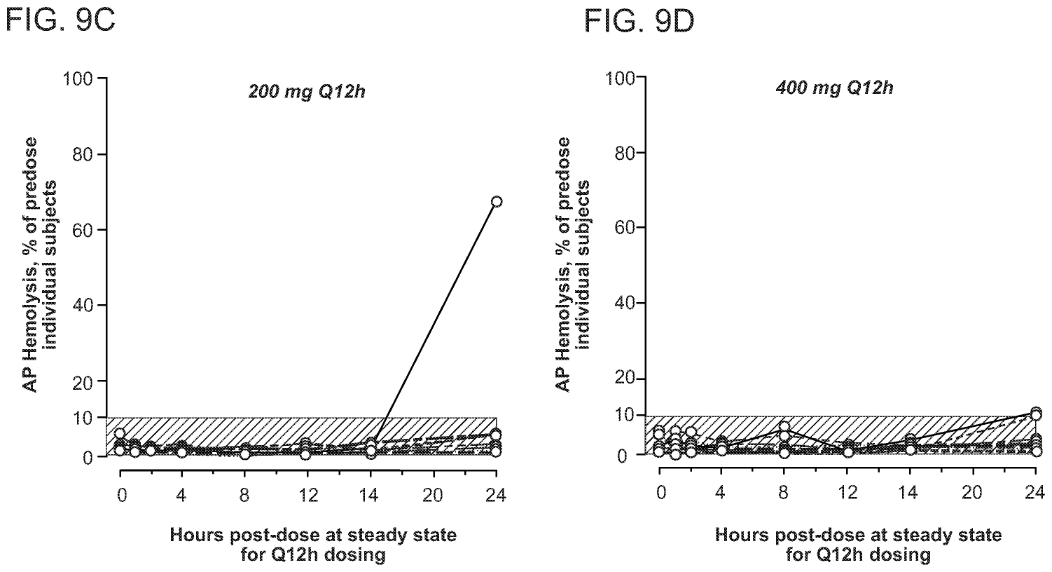

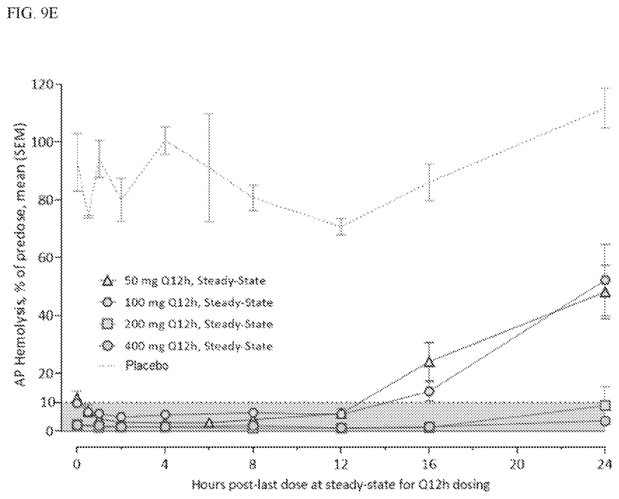

FIGS. 8A-D show individual subject values for the inhibition of AP Wieslab activity for Cohorts 1, 2, 4, and 5, respectively, of Part 2 and demonstrates dose-dependent suppression. Greater than 90% suppression of AP Wieslab activity was sustained for 12 hours after administration of the final dose on day 7 in Cohort 1 (93.2%±6.8% inhibition) and Cohort 2 (97.9%±3.0% inhibition) (FIGS. 8A-B). Greater than 90% suppression of AP Wieslab activity was sustained for over 24 hours after administration of the final dose on day 3 in Cohort 4 (95.0%±8.2% inhibition) and Cohort 5 (93.3%±15.3% inhibition) (FIGS. 8C-D). FIG. 8E shows the arithmetic mean (±SEM) of AP Wieslab activity as a percent of baseline for all subjects in Cohorts 1, 2, 4, and 5 of Part 2 at steady state of the Compound. FIGS. 9A-D show individual subject values for the inhibition of AP hemolysis for Cohorts 1, 2, 4, and 5, respectively, of Part 2 and demonstrates dose-dependent suppression. Greater than 90% suppression of AP hemolysis was sustained for 12 hours after administration of the final dose on day 7 in Cohort 1 (93.8%±5.3% inhibition) and Cohort 2 (94.1%±2.6% inhibition) (FIGS. 9A-B). Greater than 90% suppression of AP hemolysis was sustained for over 24 hours after administration of the final dose on day 3 in Cohort 4 (91.1%±20.5% inhibition) and Cohort 5 (96.4%±3.8% inhibition) (FIGS. 9C-D). FIG. 9E shows the arithmetic mean (±SEM) of AP Hemolysis activity as a percent of baseline for all subjects in Cohorts 1, 2, 4, and 5 of Part 2 at steady state of the Compound. FIGS. 8E and 9E shows ≥90% suppression of AP activity was observed for the multiple ascending doses of Cohorts 1, 2, 4, and 5 of Part 2 for 12 hours following administration, with the duration and magnitude of suppression increasing with dose. The inhibition values shown for FIGS. 8A-D and 9A-D are compared to pre-dose values and are reported as mean values (±SD of the mean) derived from all subjects in each Cohort.

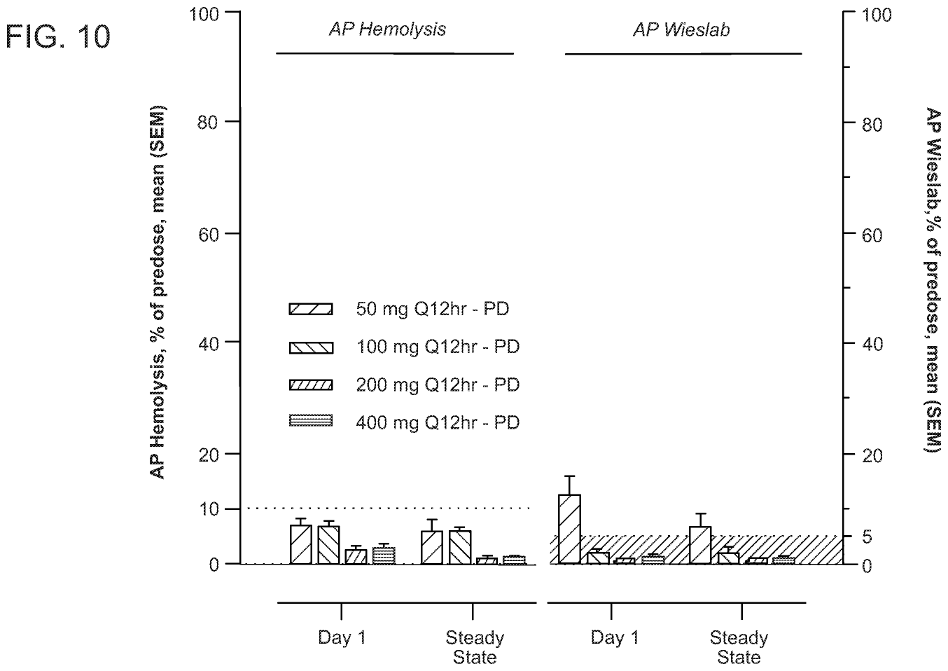

FIG. 10 shows PD profiles (AP hemolysis and AP Wieslab activity) of the Compound in Cohorts 1, 2, 4, and 5 on Day 1 (24 hours) and steady state PK troughs. Significant inhibition of AP hemolysis and AP Wieslab activity was observed in Cohorts 1, 2, 4, and 5, with Cohorts 1, 2, 4, and 5 showing greater than 90% inhibition of AP induced hemolysis at Day 1 and steady-stated PK troughs and Cohorts 2, 4, and 5 showing greater than 95% inhibition of AP Wieslab activity at Day 1 and steady-stated PK troughs. In Cohorts 4 and 5, AP activity is blocked by greater than 98% in both the AP hemolysis and AP Wieslab assays throughout the dosing interval at steady state. Importantly, the higher doses provide more consistent PD effects, which is important in PNH treatment.

PK/PD modeling of AP hemolysis and AP Wieslab data demonstrates a clear concentration-response relationship, with estimated EC₅₀ values between 21.7 and 40.7 nM. These estimates are consistent with the in vitro IC₅₀ for inhibition of proteolytic activity against C3bB of 28.1 nM.

In summary, for Part 2 Compound administered as multiple ascending oral doses from 100 to 800 mg/day for 3 to 14 days was safe and generally well tolerated in all subjects with no Compound-related safety concerns of note. Furthermore, Compound administered as multiple ascending oral doses from 100 to 800 mg/day for 3 to 14 days demonstrated clinically beneficial and dose-dependent inhibition of the AP activity.

Part 3—Multiple Ascending Oral Doses of Compound in Subjects with PNH

Part 3 is currently ongoing. Subjects with PNH are recruited into two groups: Part 3A, which includes subjects with PNH who are naïve to C5-inhibitor treatment (such as eculizumab or ravulizumab), and Part 3B, which includes subjects with PNH who are currently being treated with a C5-inhibitor (such as eculizumab or ravulizumab) and are poor responders to C5-inhibitor therapy with Compound added to existing PNH treatments. Subjects may be enrolled concurrently into Parts 3A and 3B. Up to four sequential cohorts for Parts 3A and 3B may be enrolled, each using a forced titration design for 28 days. Four subjects have been enrolled in Cohort 1 of Part 3A; Cohort 2 of Part 3A is currently enrolling subjects. Dosing regimens in Period 1 and 2 of Cohorts 3 and 4 may be the same.

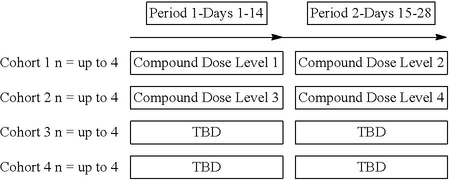

Additional Cohorts in Parts 3A and 3B may be enrolled to test higher dosing levels, 1 time per day (i.e., QD) administration, and/or administration of a single dose (administered either QD or BID) throughout the 28 days dosing period.

Subjects in Part 3A are treated with a 28-day course of study drug (Compound or placebo) administered orally in capsule dose form. For Cohorts 1 and 2 of Part 3A, the daily dose is split into 2 equal doses administered 12 hours apart (i.e., BID or Q12 hr). After 14 days of dosing at 50 mg BID, subjects in Part 3A Cohort 1 were escalated to a dose of 100 mg BID for an additional 14 days of dosing. Dosing in Part 3A Cohort 2 will start after independent data review of the data collected in Cohort 1. Dosing in Part 3A Cohort 2 will be as described for Cohort 1, except that the dose of Compound will be increased to 200 mg BID (days 1-14) and 400 mg BID (days 15-28). Dosing in Part 3B Cohort 1 may be initiated at 200 mg BID for 14 days and increased to 400 mg BID for an additional 14 days of dosing; dosing in Part 3B Cohort 2 may be further escalated. After 28 days of total dosing, subjects with positive response to treatment may continue to receive treatment for up to a total of 48 weeks, depending on responsiveness to treatment. Subjects continuing to receive Compound in the 48-week extension (i.e., subjects with positive response to treatment) will be monitored for safety and PD profiles as described herein. The 48-week extension includes the option for individual dose titrations for incomplete clinical benefit. Safety and tolerability of the Compound are evaluated as described for Part 1 and 2 herein, and plasma concentrations and PD effects of the Compound are determined as described in Parts 1 and 2 herein.

The primary endpoints of Part 3 are safety and tolerability based on laboratory and examination findings. Secondary endpoints for Part 3 include the identification of a therapeutically active dose regimen in PNH patients, determination of pharmacokinetic parameters (including, but not limited to, Cₘₐₓ, Tₘₐₓ, T_{1/2}, and AUCₜₐᵤ) and pharmacodynamics profiles (including, but not limited to, plasma factor Bb, PNH clone size, number of blood transfusions, lactate dehydrogenase activity, hemoglobin levels, bilirubin levels, absolute reticulocyte count, and haptoglobin levels) in PNH patients.

Initial results from four subjects in Cohort 1, Part 3A have been obtained. Compound administered as ascending oral doses of 50 mg BID for 14 days followed by 100 mg BID for an additional 14 days was safe and generally well tolerated for the four subjects of Cohort 1 in Part 3A. There were no study discontinuations, no serious adverse events, and no clinically significant changes is safety laboratory parameters. All four subjects reported mild headache 1-3 days after administration of Compound. All headaches were mild in severity and resolved spontaneously. No rashes were observed for the four subjects in Part 3A Cohort 1.

Table 3 below shows the baseline characteristics for the four subjects in Part 3A Cohort 1. As can be seen in Table 3, all four subjects were seriously ill with PNH. Subject 1 had a previous cerebral vein thrombosis from PNH, subject 2 required multiple red blood cell (RBC) transfusions in the year prior to screening and subject 3 was diagnosed with aplastic anemia-PNH.

Consistent with other PNH studies, subjects 1-4 showed variable pre-treatment degrees of hemolysis and anemia. Among the four subjects of Part 3A, Cohort 1, the lactate dehydrogenase (LDH) level, a sensitive marker of hemolysis, ranged over 800 to over 2400 IU/L, or 3.7 to 11 times the upper limit of normal (ULN). The degree of anemia was severe, with hemoglobin of 6.0 to 10.7 g/dL. All four subjects had elevated reticulocyte counts (130 to 285×10³ cell/μL), reflecting the bone marrow working harder to increase hemoglobin levels.

TABLE 3
Baseline characteristics for the subjects
enrolled in Part 3 A Cohort 1

| Pre-treatment | Subject | Subject | Subject | Subject |
|---|---|---|---|---|
| Characteristic* | 1 | 2 | 3 | 4 |
| PNH duration, years | 8 | 4 | 3 | 5 |
| History of aplastic anemia | No | No | Yes | No |
| History of thrombosis | Yes | No | No | No |
| LDH, IU/L | 2205 | 2497 | 835 | 1719 |
| LDH × ULN | 9.8 | 11.0 | 3.7 | 6.9 |
| Hemoglobin, g/dL | 8.2 | 7.0 | 6.0 | 10.7 |
| Reticulocytes, 10³ cell/pL | 220 | 285 | 130 | 200 |
| Total bilirubin, mg/dL | 3.33 | 1.47 | 1.12 | 0.61 |
| PNH type III erythrocyte | 89 | 41 | 49 | 49 |
| clone size, % | | | | |
| Units of RBC transfused in | 0 | 13 | 0 | 2 |
| 52 weeks prior to screening | | | | |
| Units of RBC transfused in | 0 | 2 | 0 | 0 |
| 12 weeks prior to screening | | | | |

*Laboratory values for LDH, reticulocyte count, total bilirubin, and PNH type III erythrocyte clone size pre-administration (pre-Rx) are the average of all available screening and baseline results. For hemoglobin, the pre-Rx value was the last available data prior to Day 1.

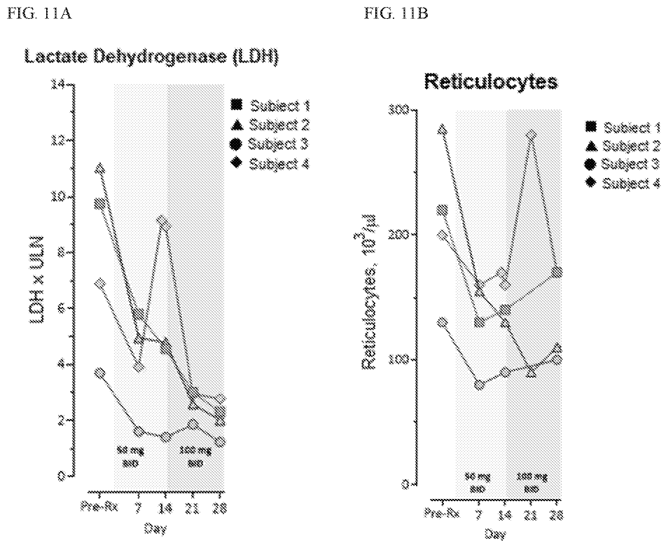

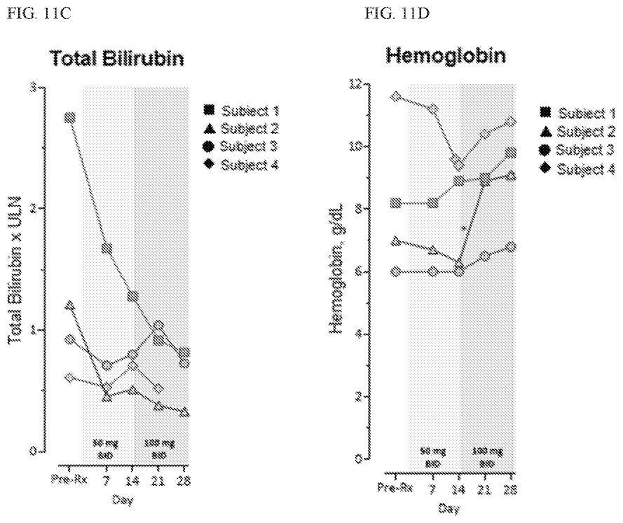

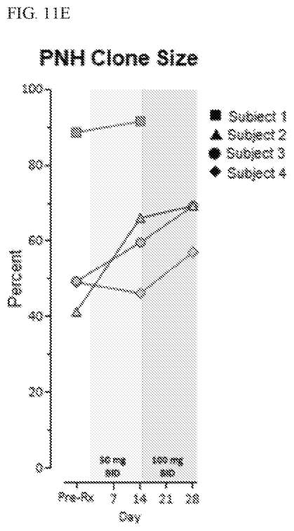

FIGS. 11A-11E show individual subject PD profiles of the Compound from 3 subjects through 28 days of administration of Compound as described above for Cohort 1 of Part 3A. All four subjects showed clinically meaningful and dose dependent reduction across all hemolysis biomarkers. Decreases in LDH (4/4 subjects) and reticulocytes (3/4 subjects), and bilirubin (2/2 subjects with elevated pretreatment values) were observed following 28 days of dosing (FIGS. 11A-11B). Total bilirubin, another marker of hemolysis in PNH, was elevated in 2 subjects at baseline and normalized following 28 days of dosing (FIG. 11C). Previous studies of complement inhibitors have shown it takes approximately 8 weeks to see stabilization of hemoglobin levels with optimized doses. Following 28 days of dosing, Hemoglobin levels were already increasing at the lowest doses of Compound (3/4 subjects) (FIG. 11D). PNH type III erythrocyte clone size also increased or was maintained in all four subjects following 28 days of dosing (FIG. 11E). As show in in Table 3, subject 2 was dependent on RBC transfusions, with a pre-administration hemoglobin level of 7.0 g/dL. Following a 2-unit RBC transfusion on Day 15, subject 2 has been transfusion-free for 6 weeks and hemoglobin levels have risen from 8.9 g/dL post-transfusion on Day 15 to 11.1 g/dL at week 8 (while continuing to receive Compound at a dose of 100 mg BID).

Subject 4 showed an initial response to treatment with Compound, followed by worsening indicators of hemolysis temporally associated with an upper respiratory tract infection (URTI; onset on Day 13). With an increase in dose to 100 mg BID and resolution of the URTI, LDH level and reticulocyte count fell and hemoglobin levels rose.

Three of four subjects reported PNH-associated symptoms, including fatigue, erectile dysfunction, headache and abdominal pain, prior to enrollment. In all cases, symptoms resolved by Day 21 of Compound administration.

The results in FIGS. 11A-E show improvement in key biomarkers of hemolysis in PNH and demonstrate the clinical utility of the Compound in treating or preventing PNH when administered as described above.

All four subjects continued into the extension. In Subjects 2 to 4 currently enrolled in the extension, the dose of Compound has been increased to 200 mg BID, a dose regimen that demonstrated ≥98% AP inhibition in healthy subjects with substantially less inter-subject variability than that observed with lower doses. Initial data from these three subjects showed that after 14 days of 200 mg BID, LDH values were 1.47×ULN in Subjects 3 and 4 (4.4×ULN in Subject 2) and hemoglobin levels were 1 mg/dL and 1.6 mg/dL higher than after 14 days of 100 mg BID in Subjects 2 and 3, respectively (with data unavailable for Subject 4).

In summary, for Cohort 1 of Part 3A Compound administered as ascending oral doses of 50 mg BID for 14 days followed by 100 mg BID for an additional 14 days was safe and generally well tolerated in all subjects with no Compound-related safety concerns of note. Furthermore, Compound administered as ascending oral doses of 50 mg BID for 14 days followed by 100 mg BID for an additional 14 days demonstrated clinically beneficial effects on key biomarkers of hemolysis in PNH.

Conclusions:

Oral dosing with compounds of the invention, potent inhibitors of human complement factor D, was safe and generally well tolerated in all subjects of Parts 1 to 3 that have completed treatment, with no Compound-related safety concerns of note. Oral dosing with compounds of the invention demonstrated linear and dose-proportional exposure, with PD parameters correlating well with the observed PK and showing rapid, sustained, and dose-dependent inhibition of AP complement activity in Parts 1 and 2. Oral dosing with compounds of the invention in Part 3 showed clinically beneficial effects on physiological parameters effected by PNH. The present disclosure demonstrates clinical utility of the Compound in treating or preventing PNH when administered as described herein.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. An oral dosage form comprising a compound selected from: 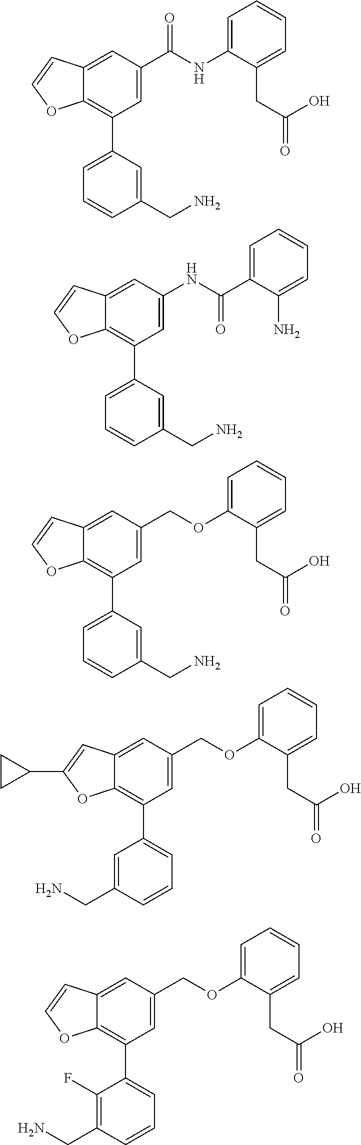 [SMILES: NCc1cccc(-c2cc(C(=O)Nc3ccccc3CC(=O)O)cc3ccoc23)c1 ; NCc1cccc(-c2cc(NC(=O)c3ccccc3N)cc3ccoc23)c1 ; NCc1cccc(-c2cc(COc3ccccc3CC(=O)O)cc3ccoc23)c1 ; NCc1cccc(-c2cc(COc3ccccc3CC(=O)O)cc3cc(C4CC4)oc23)c1 ; NCc1cccc(-c2cc(COc3ccccc3CC(=O)O)cc3ccoc23)c1F] 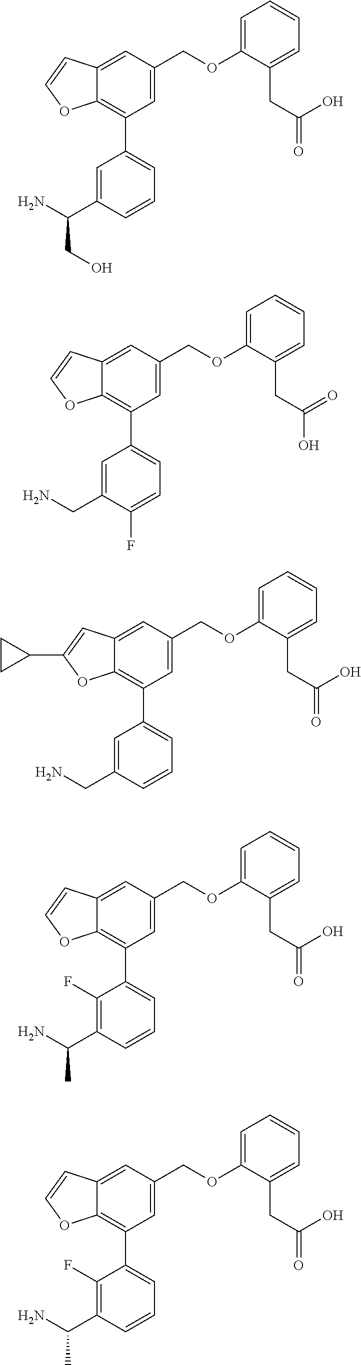 [SMILES: N[C@H](CO)c1cccc(-c2cc(COc3ccccc3CC(=O)O)cc3ccoc23)c1 ; NCc1cc(-c2cc(COc3ccccc3CC(=O)O)cc3ccoc23)ccc1F ; NCc1cccc(-c2cc(COc3ccccc3CC(=O)O)cc3cc(C4CC4)oc23)c1 ; C[C@@H](N)c1cccc(-c2cc(COc3ccccc3CC(=O)O)cc3ccoc23)c1F ; C[C@H](N)c1cccc(-c2cc(COc3ccccc3CC(=O)O)cc3ccoc23)c1F] 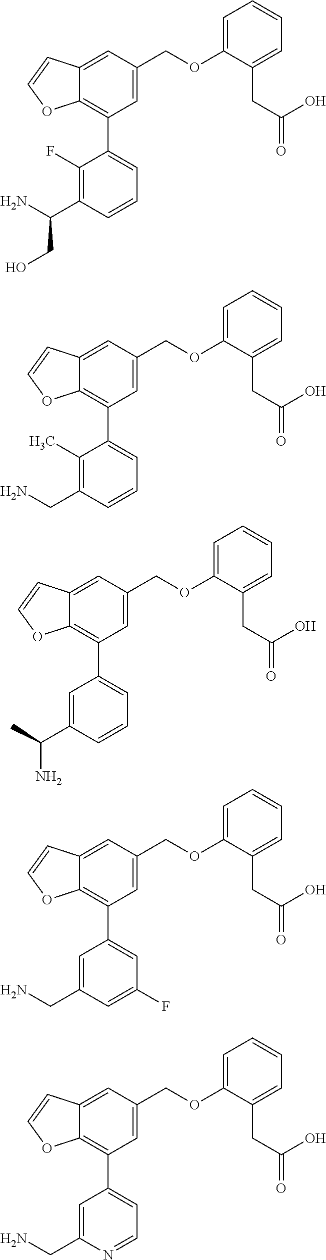 [SMILES: N[C@H](CO)c1cccc(-c2cc(COc3ccccc3CC(=O)O)cc3ccoc23)c1F ; Cc1c(CN)cccc1-c1cc(COc2ccccc2CC(=O)O)cc2ccoc12 ; C[C@H](N)c1cccc(-c2cc(COc3ccccc3CC(=O)O)cc3ccoc23)c1 ; NCc1cc(F)cc(-c2cc(COc3ccccc3CC(=O)O)cc3ccoc23)c1 ; NCc1cc(-c2cc(COc3ccccc3CC(=O)O)cc3ccoc23)ccn1] 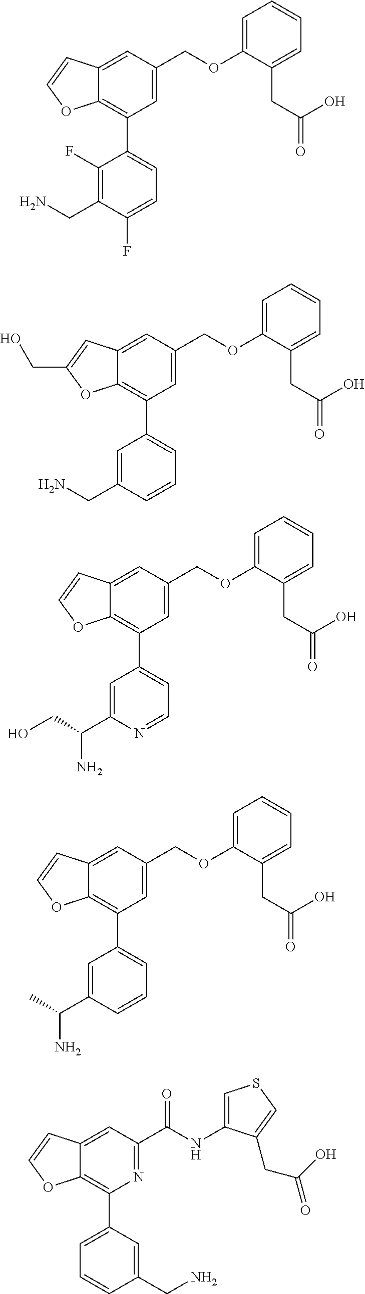 [SMILES: NCc1c(F)ccc(-c2cc(COc3ccccc3CC(=O)O)cc3ccoc23)c1F ; NCc1cccc(-c2cc(COc3ccccc3CC(=O)O)cc3cc(CO)oc23)c1 ; N[C@H](CO)c1cc(-c2cc(COc3ccccc3CC(=O)O)cc3ccoc23)ccn1 ; C[C@@H](N)c1cccc(-c2cc(COc3ccccc3CC(=O)O)cc3ccoc23)c1 ; NCc1cccc(-c2nc(C(=O)Nc3cscc3CC(=O)O)cc3ccoc23)c1] 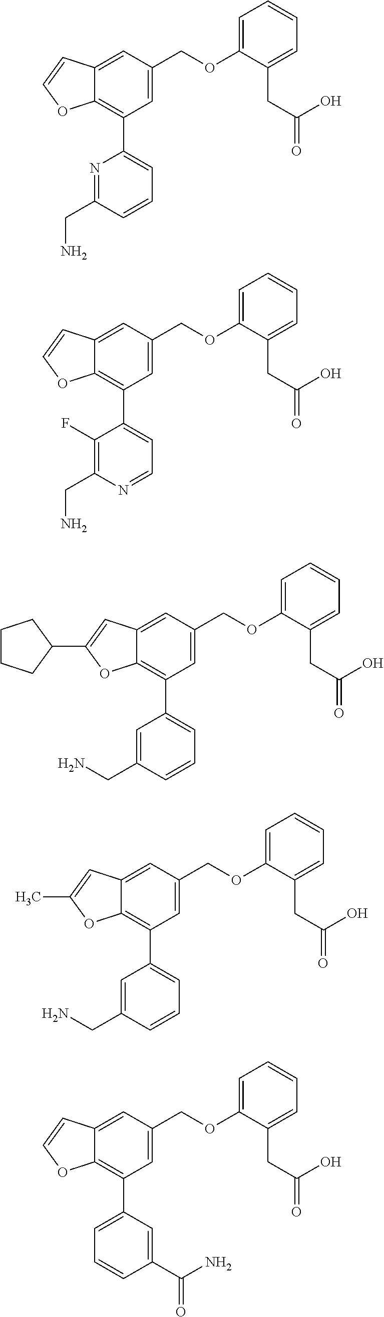 [SMILES: NCc1cccc(-c2cc(COc3ccccc3CC(=O)O)cc3ccoc23)n1 ; NCc1nccc(-c2cc(COc3ccccc3CC(=O)O)cc3ccoc23)c1F ; NCc1cccc(-c2cc(COc3ccccc3CC(=O)O)cc3cc(C4CCCC4)oc23)c1 ; Cc1cc2cc(COc3ccccc3CC(=O)O)cc(-c3cccc(CN)c3)c2o1 ; NC(=O)c1cccc(-c2cc(COc3ccccc3CC(=O)O)cc3ccoc23)c1] 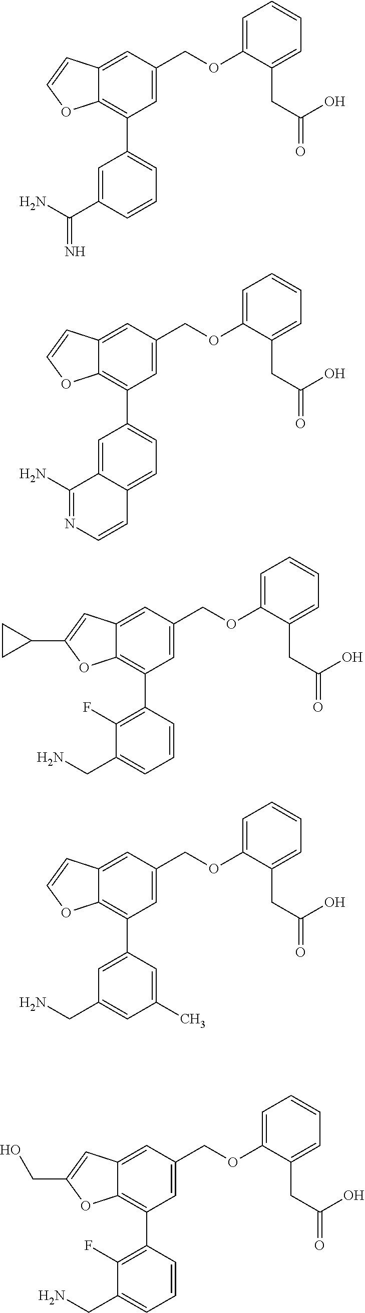 [SMILES: N=C(N)c1cccc(-c2cc(COc3ccccc3CC(=O)O)cc3ccoc23)c1 ; Nc1nccc2ccc(-c3cc(COc4ccccc4CC(=O)O)cc4ccoc34)cc12 ; NCc1cccc(-c2cc(COc3ccccc3CC(=O)O)cc3cc(C4CC4)oc23)c1F ; Cc1cc(CN)cc(-c2cc(COc3ccccc3CC(=O)O)cc3ccoc23)c1 ; NCc1cccc(-c2cc(COc3ccccc3CC(=O)O)cc3cc(CO)oc23)c1F] 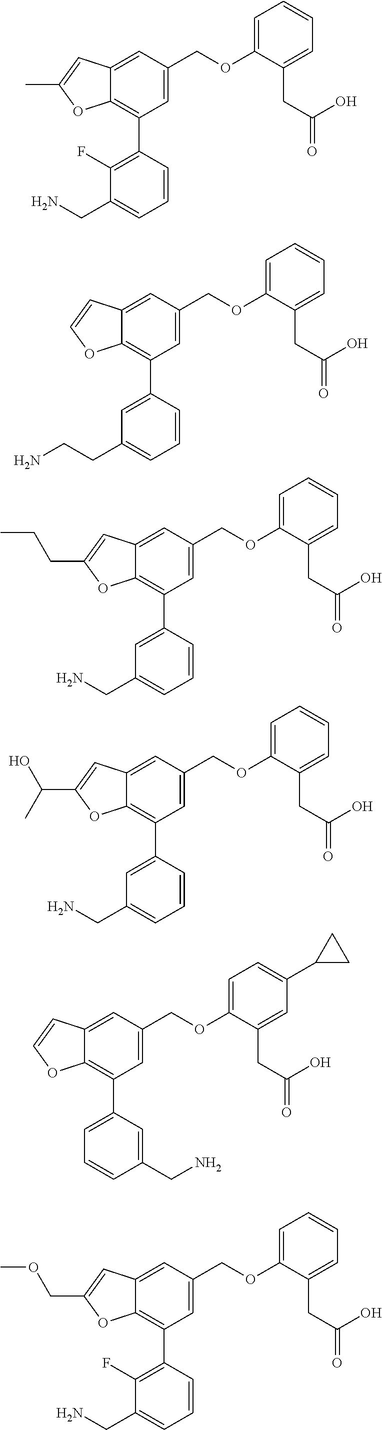 [SMILES: Cc1cc2cc(COc3ccccc3CC(=O)O)cc(-c3cccc(CN)c3F)c2o1 ; NCCc1cccc(-c2cc(COc3ccccc3CC(=O)O)cc3ccoc23)c1 ; CCCc1cc2cc(COc3ccccc3CC(=O)O)cc(-c3cccc(CN)c3)c2o1 ; CC(O)c1cc2cc(COc3ccccc3CC(=O)O)cc(-c3cccc(CN)c3)c2o1 ; NCc1cccc(-c2cc(COc3ccc(C4CC4)cc3CC(=O)O)cc3ccoc23)c1 ; COCc1cc2cc(COc3ccccc3CC(=O)O)cc(-c3cccc(CN)c3F)c2o1] 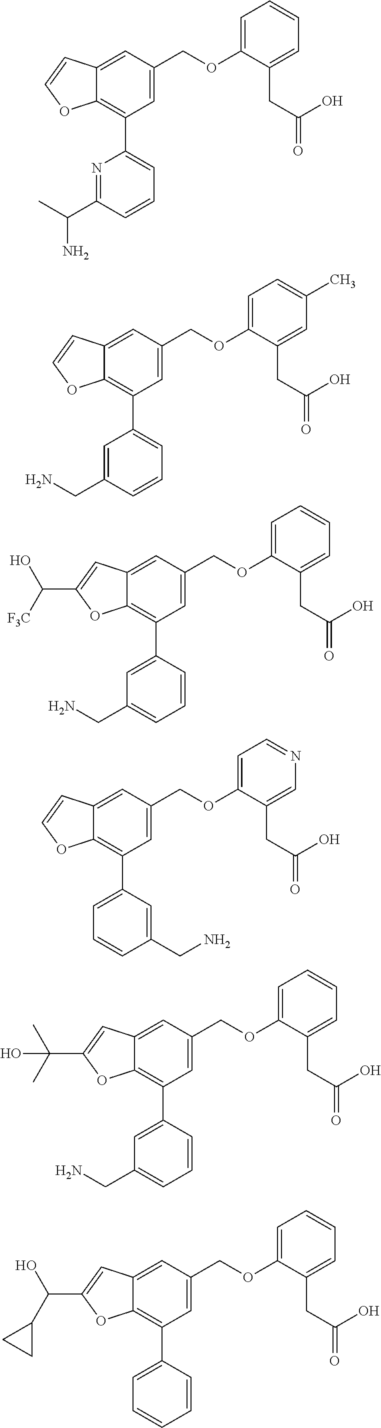 [SMILES: CC(N)c1cccc(-c2cc(COc3ccccc3CC(=O)O)cc3ccoc23)n1 ; Cc1ccc(OCc2cc(-c3cccc(CN)c3)c3occc3c2)c(CC(=O)O)c1 ; NCc1cccc(-c2cc(COc3ccccc3CC(=O)O)cc3cc(C(O)C(F)(F)F)oc23)c1 ; NCc1cccc(-c2cc(COc3ccncc3CC(=O)O)cc3ccoc23)c1 ; CC(C)(O)c1cc2cc(COc3ccccc3CC(=O)O)cc(-c3cccc(CN)c3)c2o1 ; O=C(O)Cc1ccccc1OCc1cc(-c2ccccc2)c2oc(C(O)C3CC3)cc2c1] 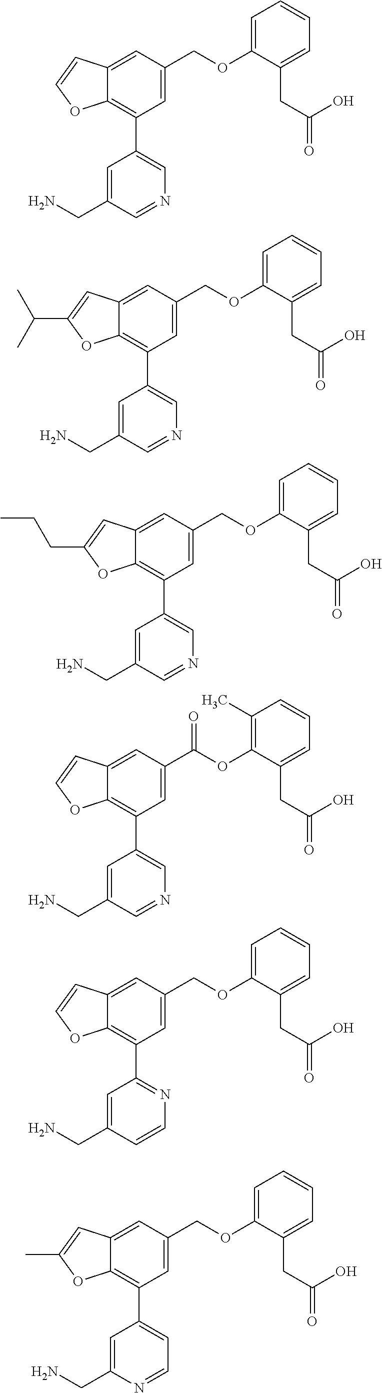 [SMILES: NCc1cncc(-c2cc(COc3ccccc3CC(=O)O)cc3ccoc23)c1 ; CC(C)c1cc2cc(COc3ccccc3CC(=O)O)cc(-c3cncc(CN)c3)c2o1 ; CCCc1cc2cc(COc3ccccc3CC(=O)O)cc(-c3cncc(CN)c3)c2o1 ; Cc1cccc(CC(=O)O)c1OC(=O)c1cc(-c2cncc(CN)c2)c2occc2c1 ; NCc1ccnc(-c2cc(COc3ccccc3CC(=O)O)cc3ccoc23)c1 ; Cc1cc2cc(COc3ccccc3CC(=O)O)cc(-c3ccnc(CN)c3)c2o1] 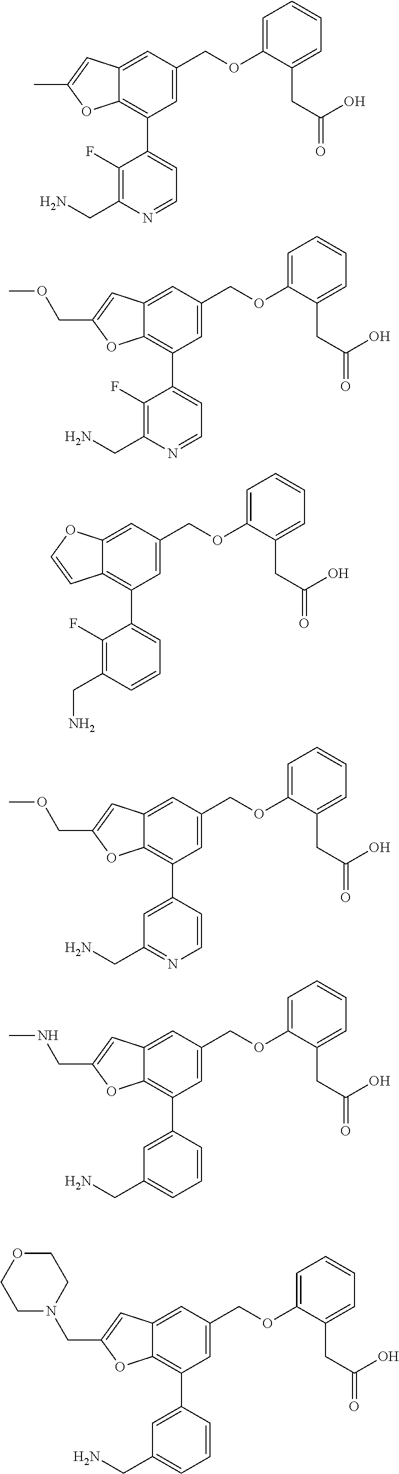 [SMILES: Cc1cc2cc(COc3ccccc3CC(=O)O)cc(-c3ccnc(CN)c3F)c2o1 ; COCc1cc2cc(COc3ccccc3CC(=O)O)cc(-c3ccnc(CN)c3F)c2o1 ; NCc1cccc(-c2cc(COc3ccccc3CC(=O)O)cc3occc23)c1F ; COCc1cc2cc(COc3ccccc3CC(=O)O)cc(-c3ccnc(CN)c3)c2o1 ; CNCc1cc2cc(COc3ccccc3CC(=O)O)cc(-c3cccc(CN)c3)c2o1 ; NCc1cccc(-c2cc(COc3ccccc3CC(=O)O)cc3cc(CN4CCOCC4)oc23)c1] 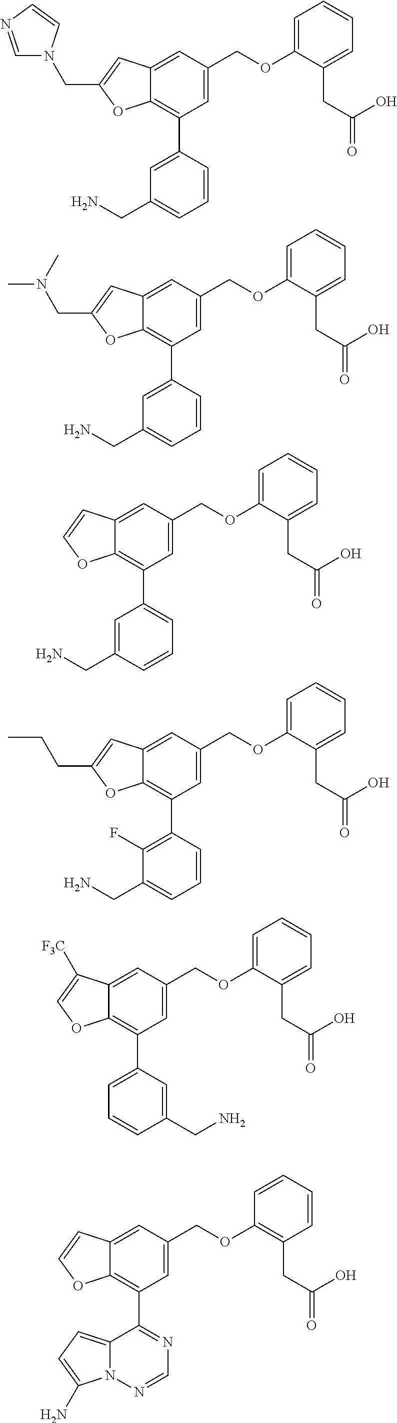 [SMILES: NCc1cccc(-c2cc(COc3ccccc3CC(=O)O)cc3cc(Cn4ccnc4)oc23)c1 ; CN(C)Cc1cc2cc(COc3ccccc3CC(=O)O)cc(-c3cccc(CN)c3)c2o1 ; NCc1cccc(-c2cc(COc3ccccc3CC(=O)O)cc3ccoc23)c1 ; CCCc1cc2cc(COc3ccccc3CC(=O)O)cc(-c3cccc(CN)c3F)c2o1 ; NCc1cccc(-c2cc(COc3ccccc3CC(=O)O)cc3c(C(F)(F)F)coc23)c1 ; Nc1ccc2c(-c3cc(COc4ccccc4CC(=O)O)cc4ccoc34)ncnn12] 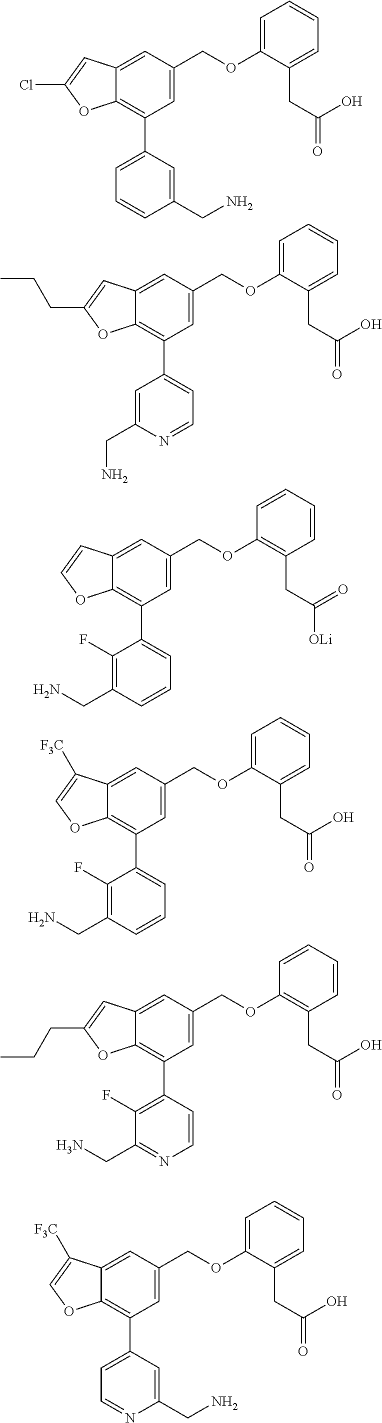 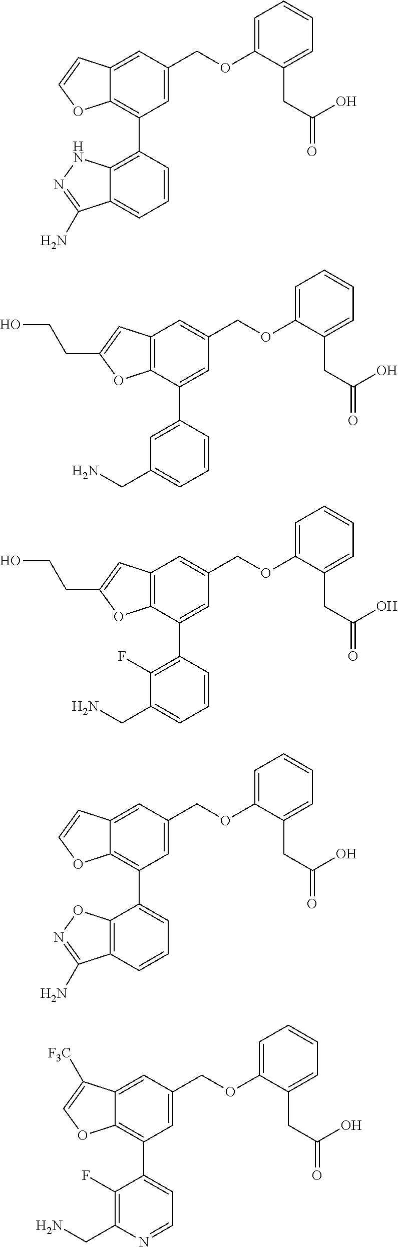 [SMILES: Nc1n[nH]c2c(-c3cc(COc4ccccc4CC(=O)O)cc4ccoc34)cccc12 ; NCc1cccc(-c2cc(COc3ccccc3CC(=O)O)cc3cc(CCO)oc23)c1 ; NCc1cccc(-c2cc(COc3ccccc3CC(=O)O)cc3cc(CCO)oc23)c1F ; Nc1noc2c(-c3cc(COc4ccccc4CC(=O)O)cc4ccoc34)cccc12 ; NCc1nccc(-c2cc(COc3ccccc3CC(=O)O)cc3c(C(F)(F)F)coc23)c1F] 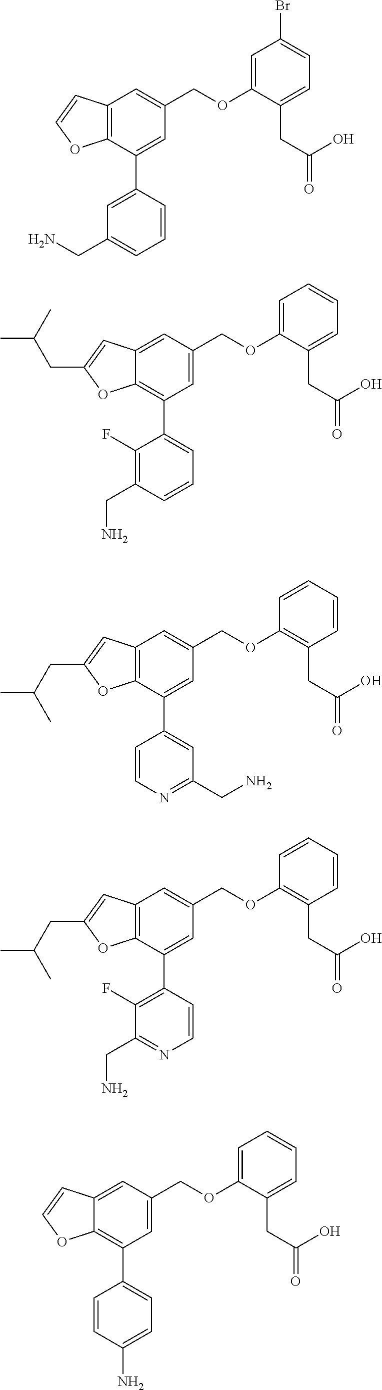 [SMILES: NCc1cccc(-c2cc(COc3cc(Br)ccc3CC(=O)O)cc3ccoc23)c1 ; CC(C)Cc1cc2cc(COc3ccccc3CC(=O)O)cc(-c3cccc(CN)c3F)c2o1 ; CC(C)Cc1cc2cc(COc3ccccc3CC(=O)O)cc(-c3ccnc(CN)c3)c2o1 ; CC(C)Cc1cc2cc(COc3ccccc3CC(=O)O)cc(-c3ccnc(CN)c3F)c2o1 ; Nc1ccc(-c2cc(COc3ccccc3CC(=O)O)cc3ccoc23)cc1] 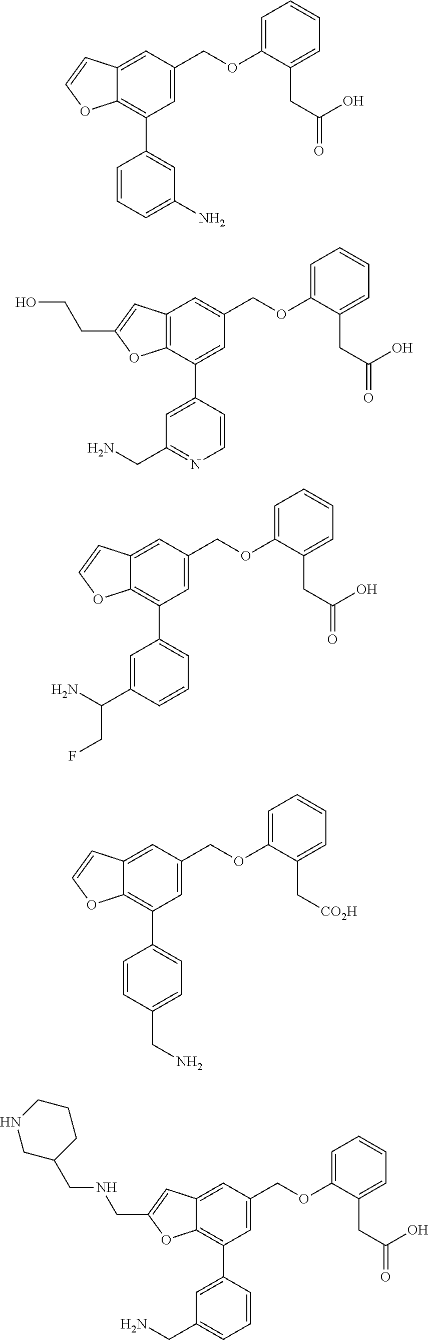 [SMILES: Nc1cccc(-c2cc(COc3ccccc3CC(=O)O)cc3ccoc23)c1 ; NCc1cc(-c2cc(COc3ccccc3CC(=O)O)cc3cc(CCO)oc23)ccn1 ; NC(CF)c1cccc(-c2cc(COc3ccccc3CC(=O)O)cc3ccoc23)c1 ; NCc1ccc(-c2cc(COc3ccccc3CC(=O)O)cc3ccoc23)cc1 ; NCc1cccc(-c2cc(COc3ccccc3CC(=O)O)cc3cc(CNCC4CCCNC4)oc23)c1] 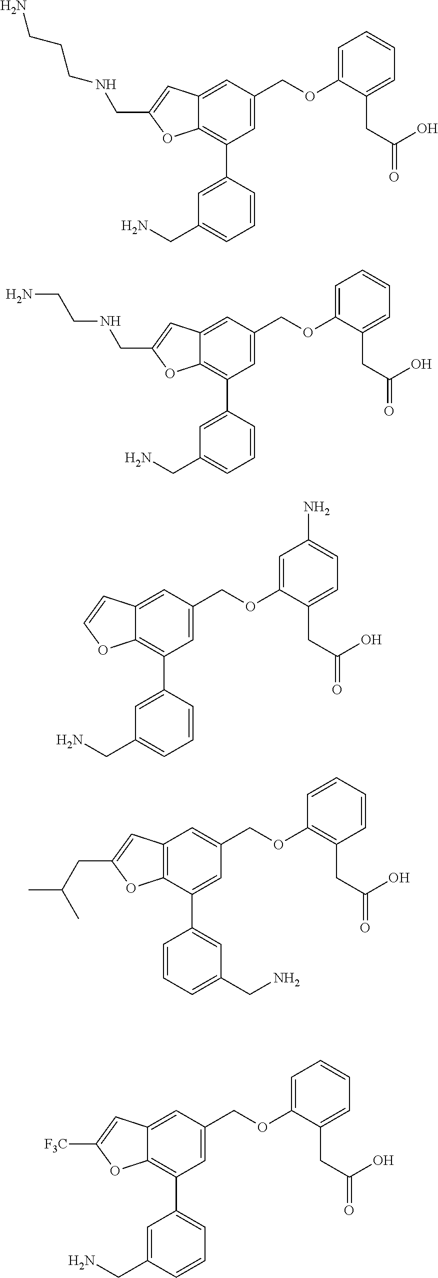 [SMILES: NCCCNCc1cc2cc(COc3ccccc3CC(=O)O)cc(-c3cccc(CN)c3)c2o1 ; NCCNCc1cc2cc(COc3ccccc3CC(=O)O)cc(-c3cccc(CN)c3)c2o1 ; NCc1cccc(-c2cc(COc3cc(N)ccc3CC(=O)O)cc3ccoc23)c1 ; CC(C)Cc1cc2cc(COc3ccccc3CC(=O)O)cc(-c3cccc(CN)c3)c2o1 ; NCc1cccc(-c2cc(COc3ccccc3CC(=O)O)cc3cc(C(F)(F)F)oc23)c1] 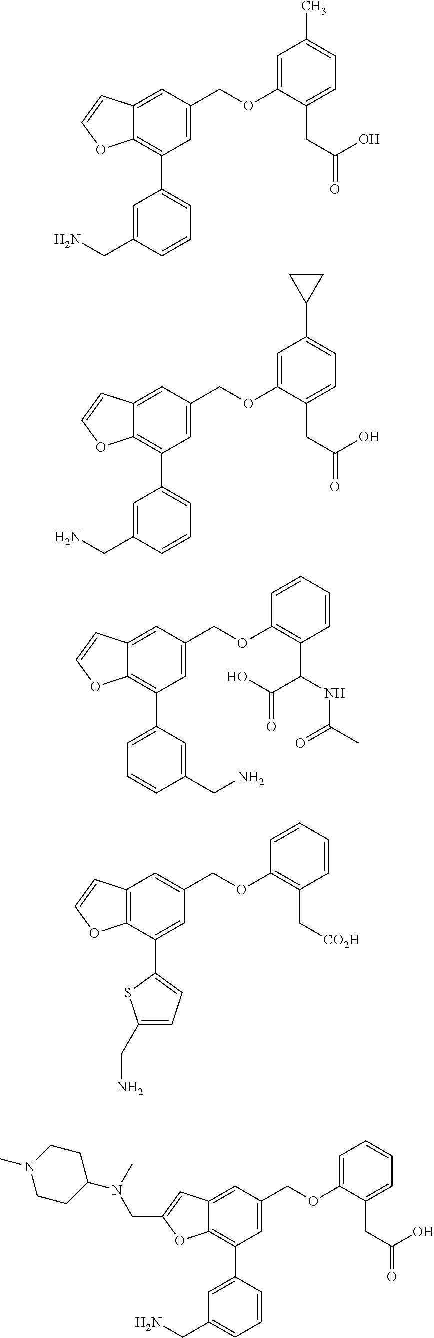 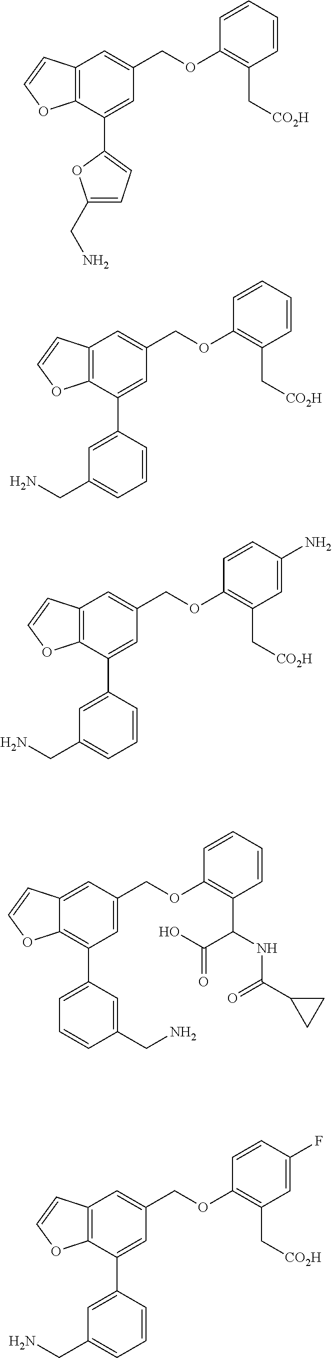 [SMILES: NCc1ccc(-c2cc(COc3ccccc3CC(=O)O)cc3ccoc23)o1 ; NCc1cccc(-c2cc(COc3ccccc3CC(=O)O)cc3ccoc23)c1 ; NCc1cccc(-c2cc(COc3ccc(N)cc3CC(=O)O)cc3ccoc23)c1 ; NCc1cccc(-c2cc(COc3ccccc3C(NC(=O)C3CC3)C(=O)O)cc3ccoc23)c1 ; NCc1cccc(-c2cc(COc3ccc(F)cc3CC(=O)O)cc3ccoc23)c1] 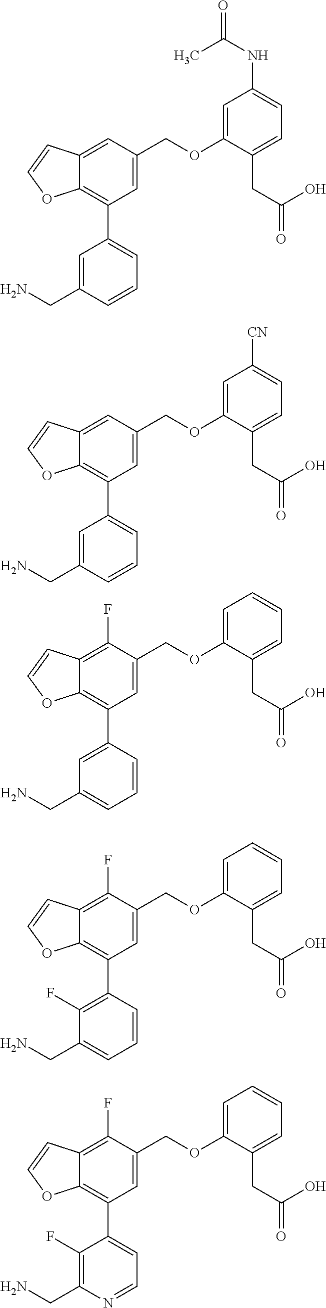 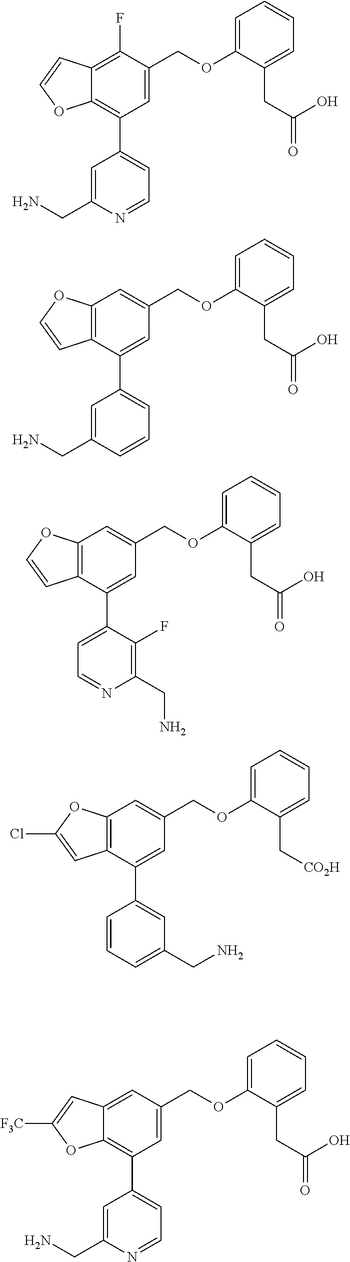 [SMILES: NCc1cc(-c2cc(COc3ccccc3CC(=O)O)c(F)c3ccoc23)ccn1 ; NCc1cccc(-c2cc(COc3ccccc3CC(=O)O)cc3occc23)c1 ; NCc1nccc(-c2cc(COc3ccccc3CC(=O)O)cc3occc23)c1F ; NCc1cccc(-c2cc(COc3ccccc3CC(=O)O)cc3oc(Cl)cc23)c1 ; NCc1cc(-c2cc(COc3ccccc3CC(=O)O)cc3cc(C(F)(F)F)oc23)ccn1] 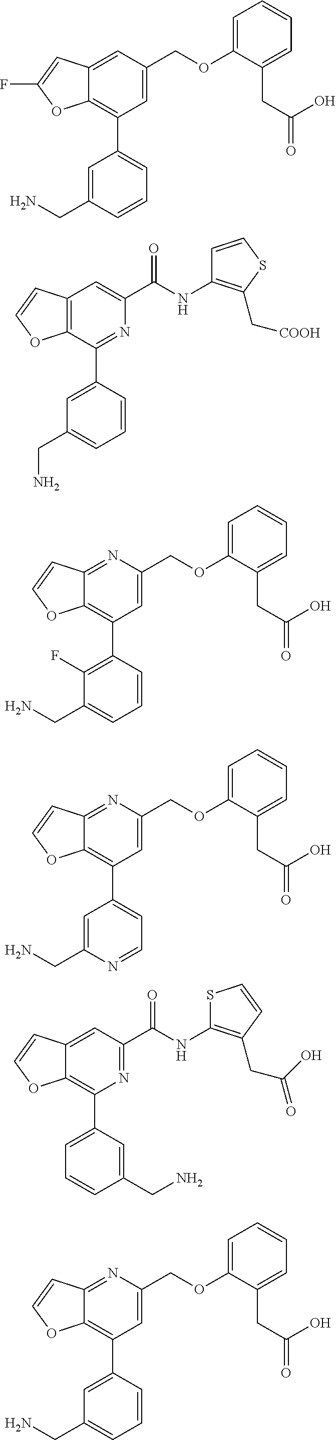 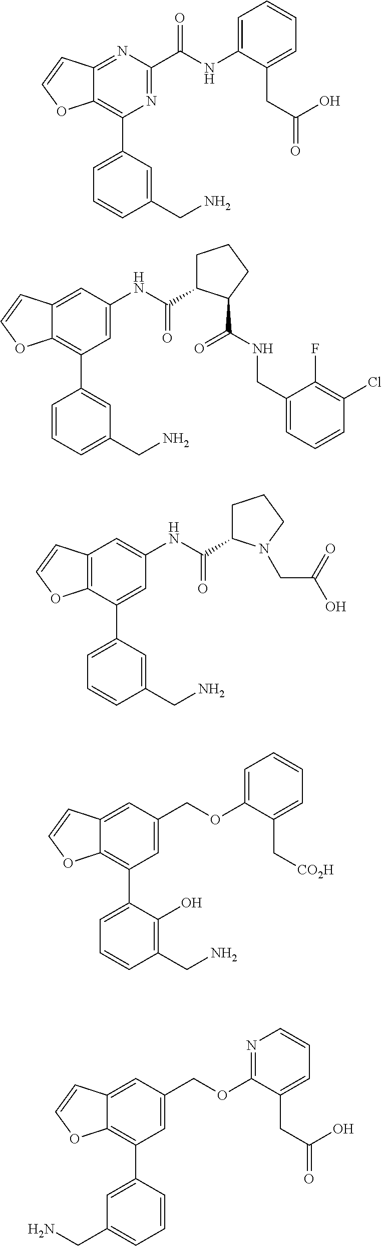 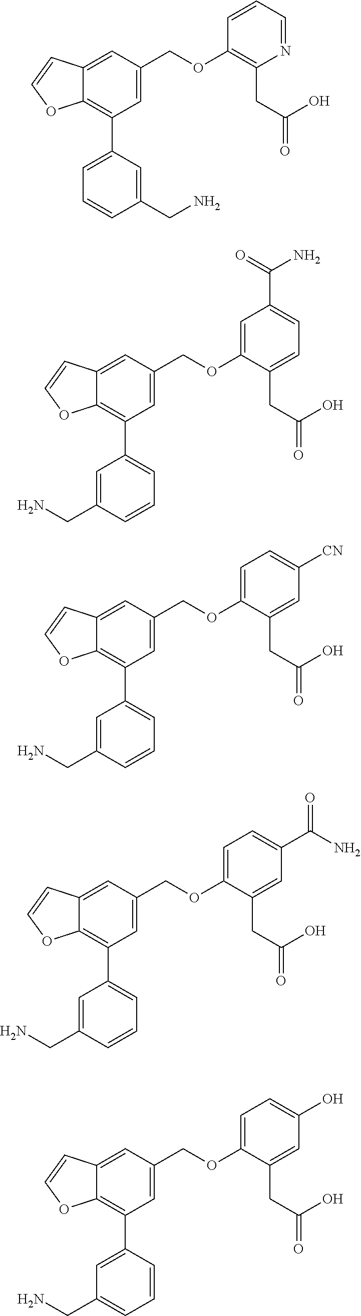 [SMILES: NCc1cccc(-c2cc(COc3cccnc3CC(=O)O)cc3ccoc23)c1 ; NCc1cccc(-c2cc(COc3cc(C(N)=O)ccc3CC(=O)O)cc3ccoc23)c1 ; N#Cc1ccc(OCc2cc(-c3cccc(CN)c3)c3occc3c2)c(CC(=O)O)c1 ; NCc1cccc(-c2cc(COc3ccc(C(N)=O)cc3CC(=O)O)cc3ccoc23)c1 ; NCc1cccc(-c2cc(COc3ccc(O)cc3CC(=O)O)cc3ccoc23)c1] 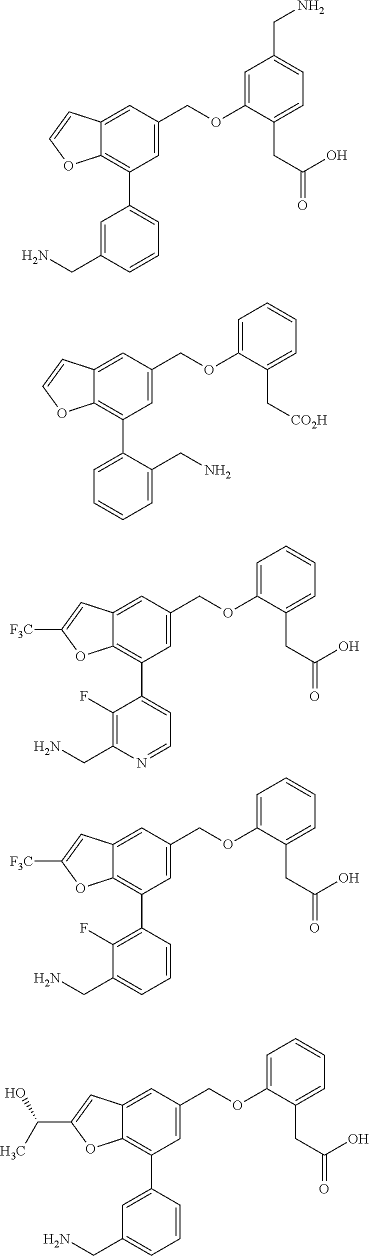 [SMILES: NCc1cccc(-c2cc(COc3cc(CN)ccc3CC(=O)O)cc3ccoc23)c1 ; NCc1ccccc1-c1cc(COc2ccccc2CC(=O)O)cc2ccoc12 ; NCc1nccc(-c2cc(COc3ccccc3CC(=O)O)cc3cc(C(F)(F)F)oc23)c1F ; NCc1cccc(-c2cc(COc3ccccc3CC(=O)O)cc3cc(C(F)(F)F)oc23)c1F ; C[C@H](O)c1cc2cc(COc3ccccc3CC(=O)O)cc(-c3cccc(CN)c3)c2o1] 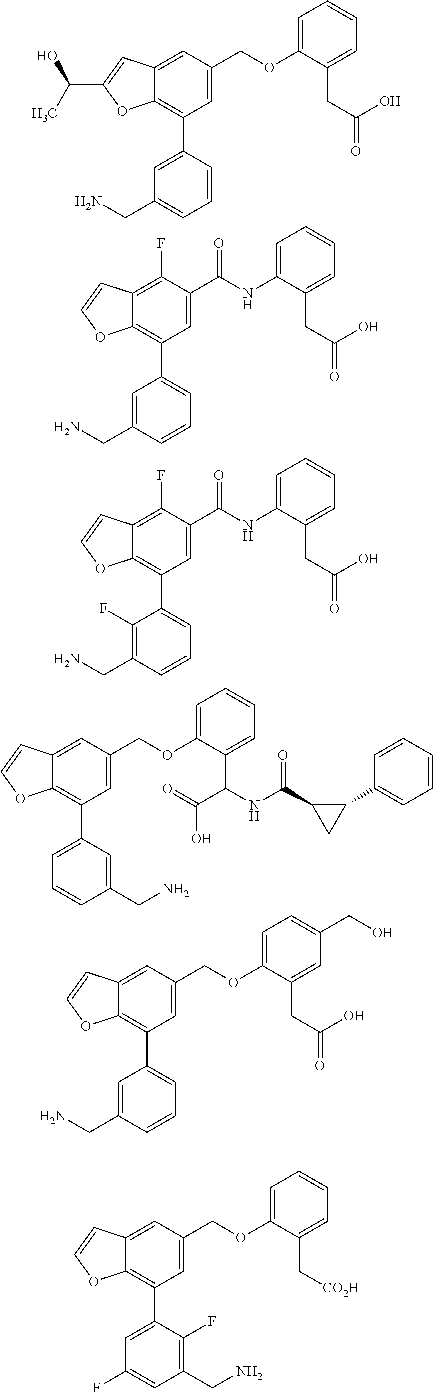 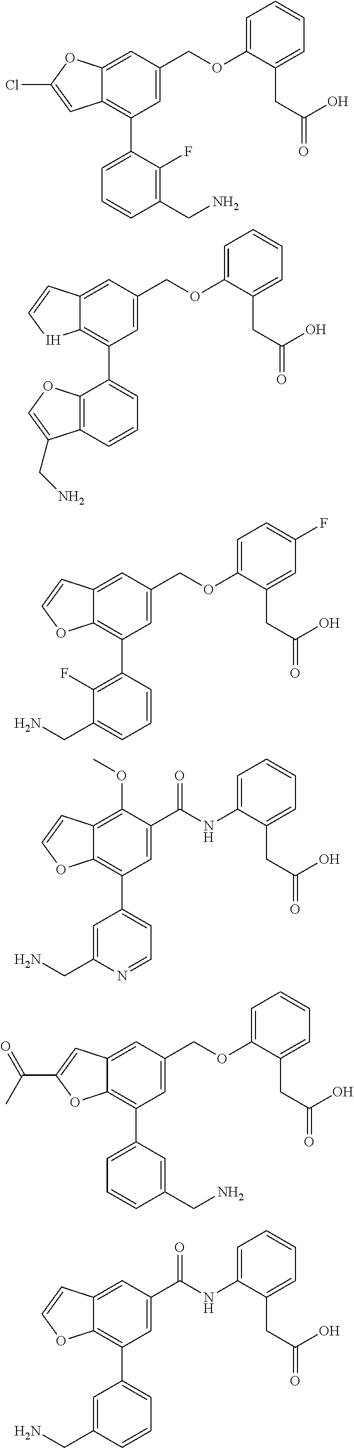 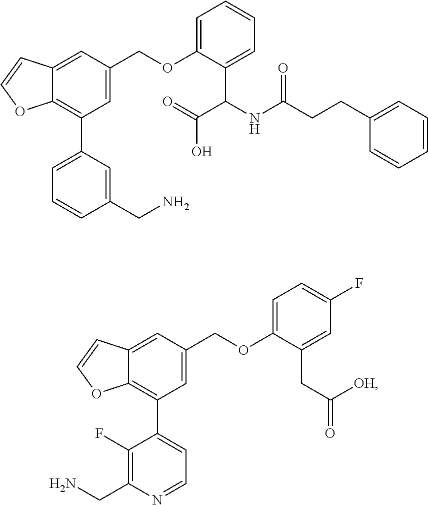 [SMILES: NCc1cccc(-c2cc(COc3ccccc3C(NC(=O)CCc3ccccc3)C(=O)O)cc3ccoc23)c1 ; NCc1nccc(-c2cc(COc3ccc(F)cc3CC(=O)O)cc3ccoc23)c1F]
or a pharmaceutically acceptable salt or prodrug thereof; and a pharmaceutically acceptable carrier.

2. The oral dosage form of claim 1, comprising about 1 mg to about 1500 mg of the compound or a pharmaceutically acceptable salt or prodrug thereof.

3. The oral dosage form of claim 1, comprising about 1 mg to about 1200 mg of the compound or a pharmaceutically acceptable salt or prodrug thereof.

4. The oral dosage form of claim 1, comprising about 1 mg to about 1000 mg of the compound or a pharmaceutically acceptable salt or prodrug thereof.

5. The oral dosage form of claim 1, comprising about 1 mg to about 800 mg of the compound or a pharmaceutically acceptable salt or prodrug thereof.

6. The oral dosage form of claim 1, comprising about 1 mg to about 600 mg of the compound or a pharmaceutically acceptable salt or prodrug thereof.

7. The oral dosage form of claim 1, comprising about 1 mg to about 400 mg of the compound or a pharmaceutically acceptable salt or prodrug thereof.

8. The oral dosage form of claim 1, comprising about 1 mg to about 300 mg of the compound or a pharmaceutically acceptable salt or prodrug thereof.

9. The oral dosage form of claim 1, comprising about 1 mg to about 200 mg of the compound or a pharmaceutically acceptable salt or prodrug thereof.

10. The oral dosage form of claim 1, comprising about 1 mg to about 100 mg of the compound or a pharmaceutically acceptable salt or prodrug thereof.

11. The oral dosage form of claim 1, comprising about 10 mg of the compound or a pharmaceutically acceptable salt or prodrug thereof.

12. The oral dosage form of claim 1, comprising about 30 mg of the compound or a pharmaceutically acceptable salt or prodrug thereof.

13. The oral dosage form of claim 1, comprising about 100 mg of the compound or a pharmaceutically acceptable salt or prodrug thereof.

14. The oral dosage form of claim 1, comprising about 200 mg of the compound or a pharmaceutically acceptable salt or prodrug thereof.

15. The oral dosage form of claim 1, comprising about 300 mg of the compound or a pharmaceutically acceptable salt or prodrug thereof.

16. The oral dosage form of claim 1, comprising about 400 mg of the compound or a pharmaceutically acceptable salt or prodrug thereof.

17. The oral dosage form of claim 1, comprising about 600 mg of the compound or a pharmaceutically acceptable salt or prodrug thereof.

18. The oral dosage form of claim 1, comprising about 800 mg of the compound or a pharmaceutically acceptable salt or prodrug thereof.

19. The oral dosage form of claim 1, comprising about 1000 mg of the compound or a pharmaceutically acceptable salt or prodrug thereof.

20. The oral dosage form of claim 1, comprising about 1200 mg of the compound or a pharmaceutically acceptable salt or prodrug thereof.

21. The oral dosage form of any one of claims 1-20, wherein the oral dosage form is a capsule.

22. The oral dosage form of any one of claims 1-20, wherein the oral dosage form is a tablet.

23. The oral dosage form of claim 22, wherein the tablet is a coated tablet.

24. A method of treating or preventing a disease or condition characterized by aberrant complement system activity, comprising orally administering to a patient in need thereof a therapeutically effective amount of a compound selected from: 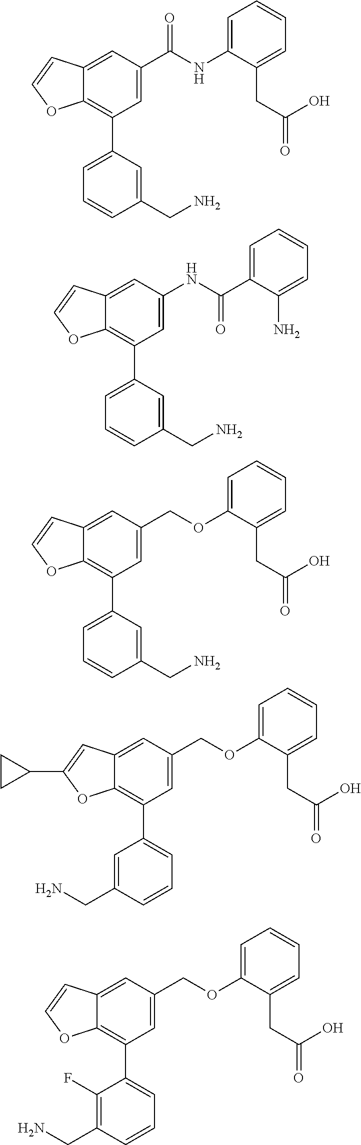 [SMILES: NCc1cccc(-c2cc(C(=O)Nc3ccccc3CC(=O)O)cc3ccoc23)c1 ; NCc1cccc(-c2cc(NC(=O)c3ccccc3N)cc3ccoc23)c1 ; NCc1cccc(-c2cc(COc3ccccc3CC(=O)O)cc3ccoc23)c1 ; NCc1cccc(-c2cc(COc3ccccc3CC(=O)O)cc3cc(C4CC4)oc23)c1 ; NCc1cccc(-c2cc(COc3ccccc3CC(=O)O)cc3ccoc23)c1F] 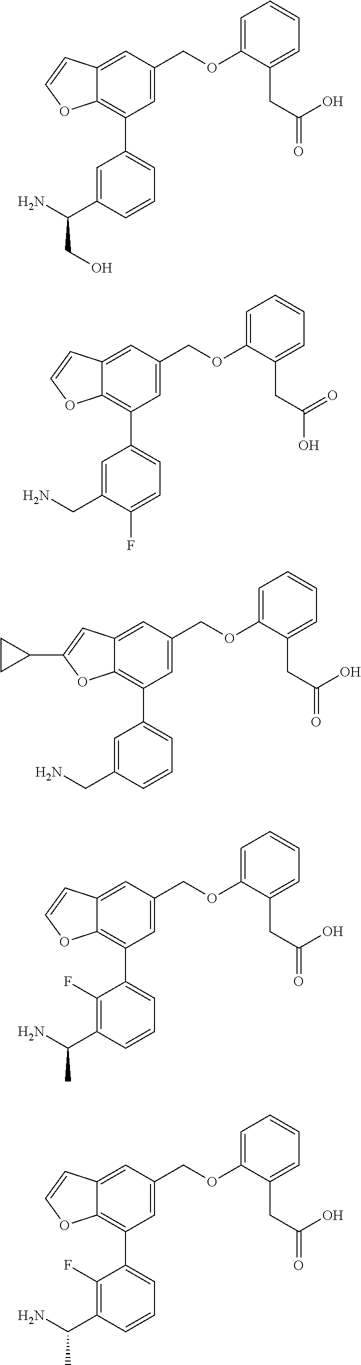 [SMILES: N[C@H](CO)c1cccc(-c2cc(COc3ccccc3CC(=O)O)cc3ccoc23)c1 ; NCc1cc(-c2cc(COc3ccccc3CC(=O)O)cc3ccoc23)ccc1F ; NCc1cccc(-c2cc(COc3ccccc3CC(=O)O)cc3cc(C4CC4)oc23)c1 ; C[C@@H](N)c1cccc(-c2cc(COc3ccccc3CC(=O)O)cc3ccoc23)c1F ; C[C@H](N)c1cccc(-c2cc(COc3ccccc3CC(=O)O)cc3ccoc23)c1F] 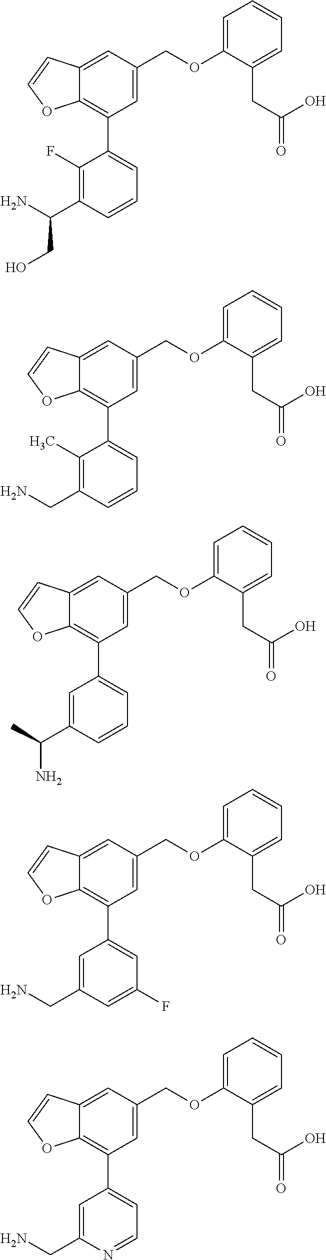 [SMILES: N[C@H](CO)c1cccc(-c2cc(COc3ccccc3CC(=O)O)cc3ccoc23)c1F ; Cc1c(CN)cccc1-c1cc(COc2ccccc2CC(=O)O)cc2ccoc12 ; C[C@H](N)c1cccc(-c2cc(COc3ccccc3CC(=O)O)cc3ccoc23)c1 ; NCc1cc(F)cc(-c2cc(COc3ccccc3CC(=O)O)cc3ccoc23)c1 ; NCc1cc(-c2cc(COc3ccccc3CC(=O)O)cc3ccoc23)ccn1] 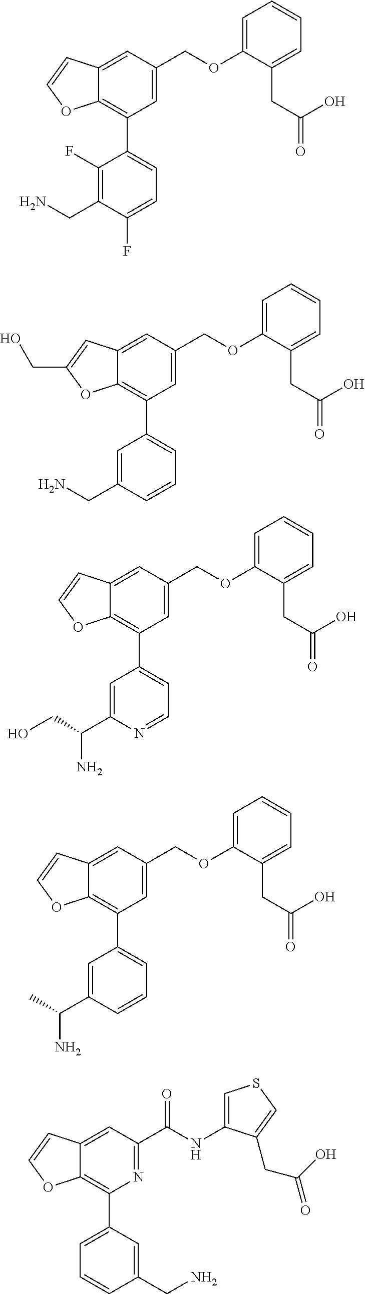 [SMILES: NCc1c(F)ccc(-c2cc(COc3ccccc3CC(=O)O)cc3ccoc23)c1F ; NCc1cccc(-c2cc(COc3ccccc3CC(=O)O)cc3cc(CO)oc23)c1 ; N[C@H](CO)c1cc(-c2cc(COc3ccccc3CC(=O)O)cc3ccoc23)ccn1 ; C[C@@H](N)c1cccc(-c2cc(COc3ccccc3CC(=O)O)cc3ccoc23)c1 ; NCc1cccc(-c2nc(C(=O)Nc3cscc3CC(=O)O)cc3ccoc23)c1] 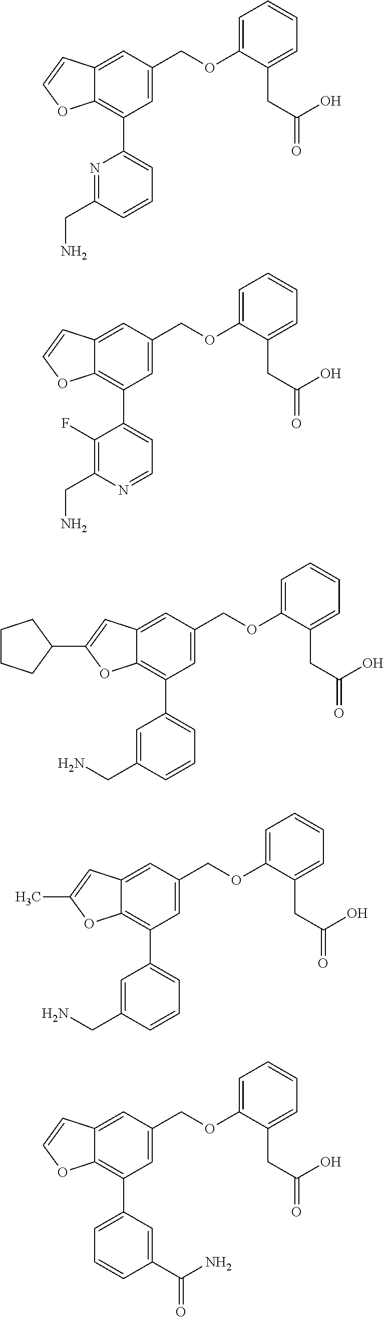 [SMILES: NCc1cccc(-c2cc(COc3ccccc3CC(=O)O)cc3ccoc23)n1 ; NCc1nccc(-c2cc(COc3ccccc3CC(=O)O)cc3ccoc23)c1F ; NCc1cccc(-c2cc(COc3ccccc3CC(=O)O)cc3cc(C4CCCC4)oc23)c1 ; Cc1cc2cc(COc3ccccc3CC(=O)O)cc(-c3cccc(CN)c3)c2o1 ; NC(=O)c1cccc(-c2cc(COc3ccccc3CC(=O)O)cc3ccoc23)c1] 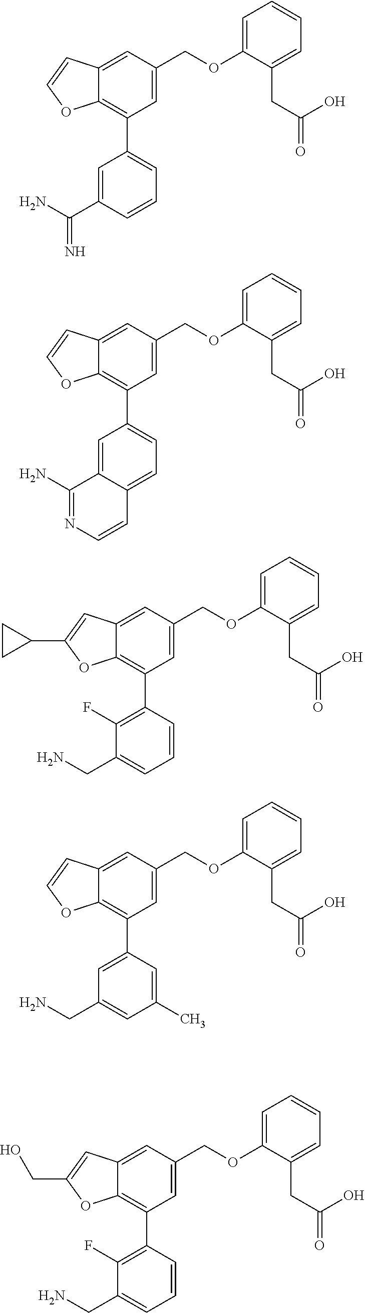 [SMILES: N=C(N)c1cccc(-c2cc(COc3ccccc3CC(=O)O)cc3ccoc23)c1 ; Nc1nccc2ccc(-c3cc(COc4ccccc4CC(=O)O)cc4ccoc34)cc12 ; NCc1cccc(-c2cc(COc3ccccc3CC(=O)O)cc3cc(C4CC4)oc23)c1F ; Cc1cc(CN)cc(-c2cc(COc3ccccc3CC(=O)O)cc3ccoc23)c1 ; NCc1cccc(-c2cc(COc3ccccc3CC(=O)O)cc3cc(CO)oc23)c1F] 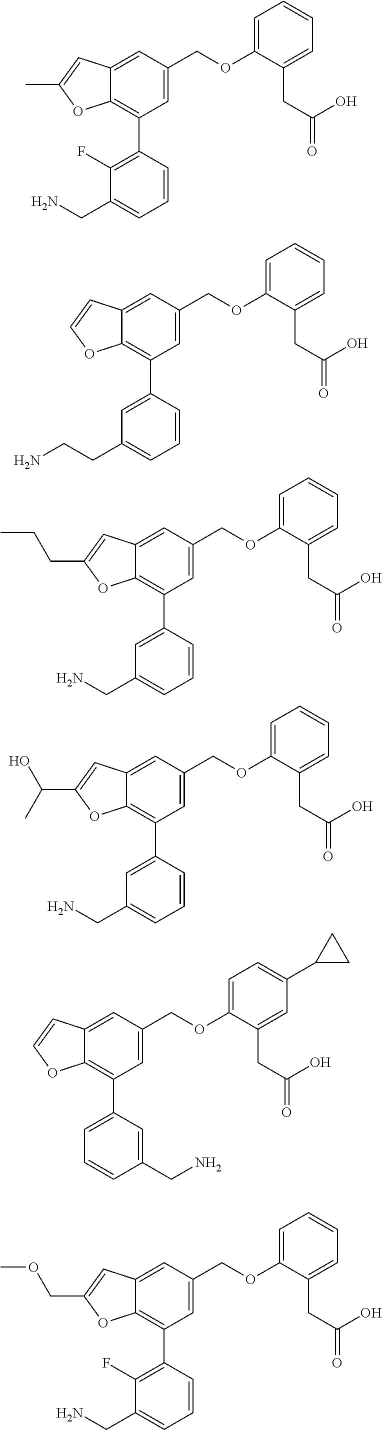 [SMILES: Cc1cc2cc(COc3ccccc3CC(=O)O)cc(-c3cccc(CN)c3F)c2o1 ; NCCc1cccc(-c2cc(COc3ccccc3CC(=O)O)cc3ccoc23)c1 ; CCCc1cc2cc(COc3ccccc3CC(=O)O)cc(-c3cccc(CN)c3)c2o1 ; CC(O)c1cc2cc(COc3ccccc3CC(=O)O)cc(-c3cccc(CN)c3)c2o1 ; NCc1cccc(-c2cc(COc3ccc(C4CC4)cc3CC(=O)O)cc3ccoc23)c1 ; COCc1cc2cc(COc3ccccc3CC(=O)O)cc(-c3cccc(CN)c3F)c2o1] 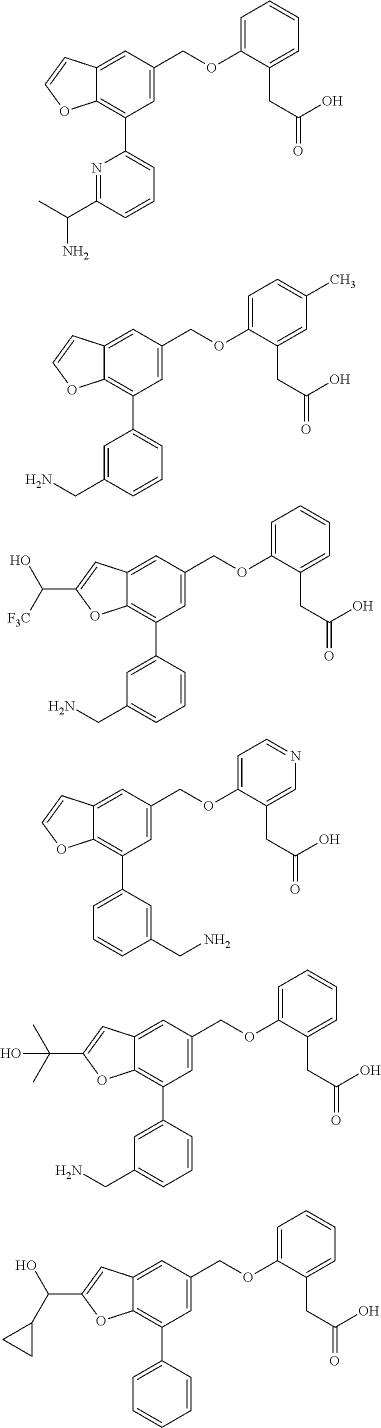 [SMILES: CC(N)c1cccc(-c2cc(COc3ccccc3CC(=O)O)cc3ccoc23)n1 ; Cc1ccc(OCc2cc(-c3cccc(CN)c3)c3occc3c2)c(CC(=O)O)c1 ; NCc1cccc(-c2cc(COc3ccccc3CC(=O)O)cc3cc(C(O)C(F)(F)F)oc23)c1 ; NCc1cccc(-c2cc(COc3ccncc3CC(=O)O)cc3ccoc23)c1 ; CC(C)(O)c1cc2cc(COc3ccccc3CC(=O)O)cc(-c3cccc(CN)c3)c2o1 ; O=C(O)Cc1ccccc1OCc1cc(-c2ccccc2)c2oc(C(O)C3CC3)cc2c1] 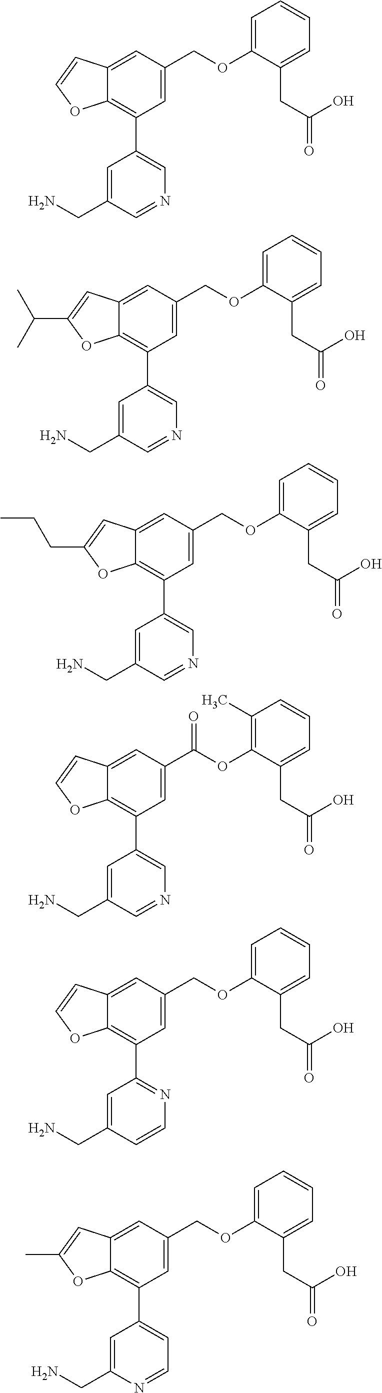 [SMILES: NCc1cncc(-c2cc(COc3ccccc3CC(=O)O)cc3ccoc23)c1 ; CC(C)c1cc2cc(COc3ccccc3CC(=O)O)cc(-c3cncc(CN)c3)c2o1 ; CCCc1cc2cc(COc3ccccc3CC(=O)O)cc(-c3cncc(CN)c3)c2o1 ; Cc1cccc(CC(=O)O)c1OC(=O)c1cc(-c2cncc(CN)c2)c2occc2c1 ; NCc1ccnc(-c2cc(COc3ccccc3CC(=O)O)cc3ccoc23)c1 ; Cc1cc2cc(COc3ccccc3CC(=O)O)cc(-c3ccnc(CN)c3)c2o1] 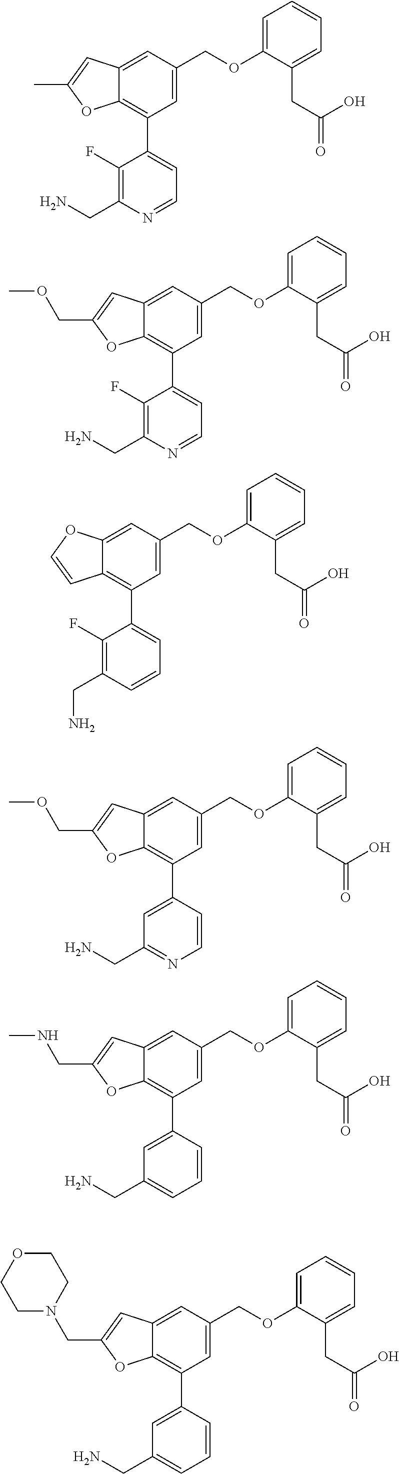 [SMILES: Cc1cc2cc(COc3ccccc3CC(=O)O)cc(-c3ccnc(CN)c3F)c2o1 ; COCc1cc2cc(COc3ccccc3CC(=O)O)cc(-c3ccnc(CN)c3F)c2o1 ; NCc1cccc(-c2cc(COc3ccccc3CC(=O)O)cc3occc23)c1F ; COCc1cc2cc(COc3ccccc3CC(=O)O)cc(-c3ccnc(CN)c3)c2o1 ; CNCc1cc2cc(COc3ccccc3CC(=O)O)cc(-c3cccc(CN)c3)c2o1 ; NCc1cccc(-c2cc(COc3ccccc3CC(=O)O)cc3cc(CN4CCOCC4)oc23)c1] 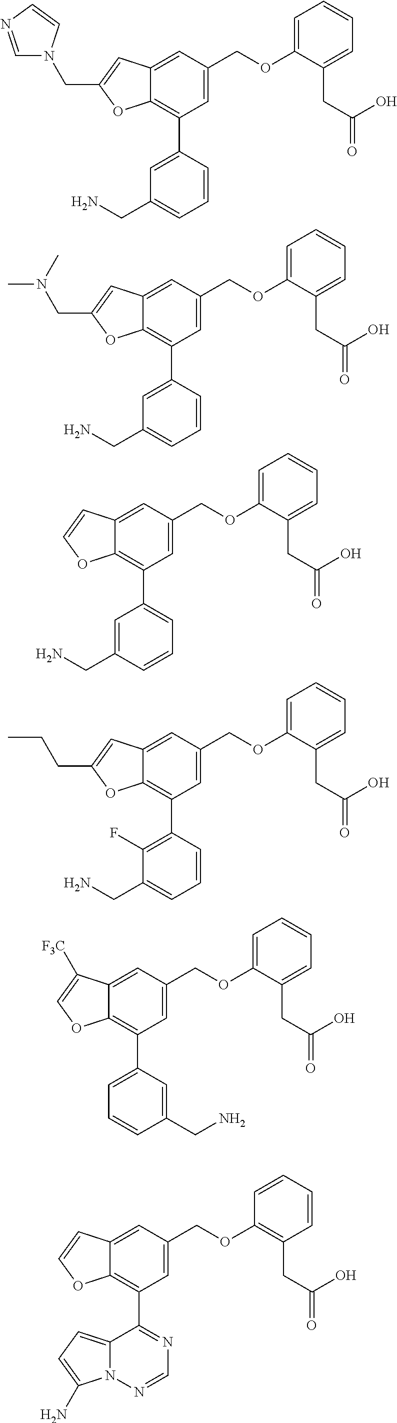 [SMILES: NCc1cccc(-c2cc(COc3ccccc3CC(=O)O)cc3cc(Cn4ccnc4)oc23)c1 ; CN(C)Cc1cc2cc(COc3ccccc3CC(=O)O)cc(-c3cccc(CN)c3)c2o1 ; NCc1cccc(-c2cc(COc3ccccc3CC(=O)O)cc3ccoc23)c1 ; CCCc1cc2cc(COc3ccccc3CC(=O)O)cc(-c3cccc(CN)c3F)c2o1 ; NCc1cccc(-c2cc(COc3ccccc3CC(=O)O)cc3c(C(F)(F)F)coc23)c1 ; Nc1ccc2c(-c3cc(COc4ccccc4CC(=O)O)cc4ccoc34)ncnn12] 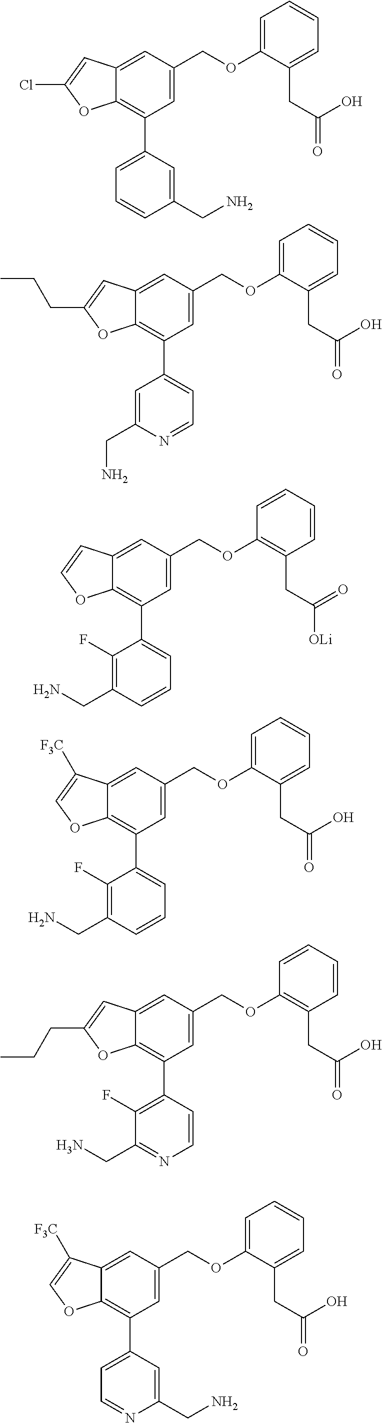 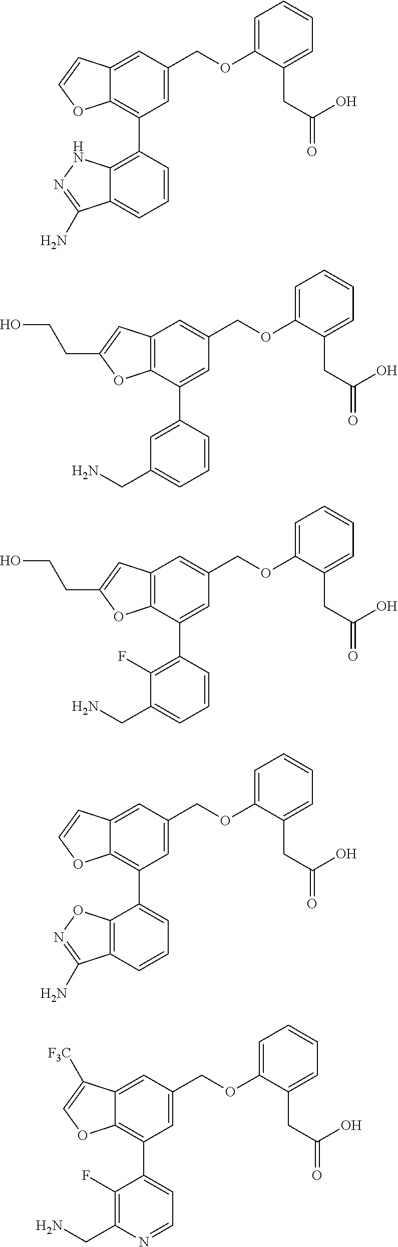 [SMILES: Nc1n[nH]c2c(-c3cc(COc4ccccc4CC(=O)O)cc4ccoc34)cccc12 ; NCc1cccc(-c2cc(COc3ccccc3CC(=O)O)cc3cc(CCO)oc23)c1 ; NCc1cccc(-c2cc(COc3ccccc3CC(=O)O)cc3cc(CCO)oc23)c1F ; Nc1noc2c(-c3cc(COc4ccccc4CC(=O)O)cc4ccoc34)cccc12 ; NCc1nccc(-c2cc(COc3ccccc3CC(=O)O)cc3c(C(F)(F)F)coc23)c1F] 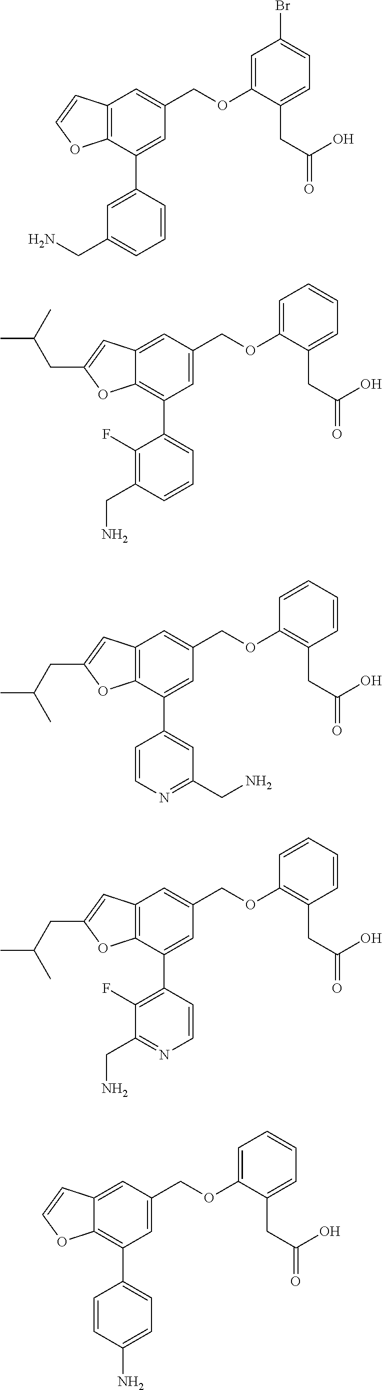 [SMILES: NCc1cccc(-c2cc(COc3cc(Br)ccc3CC(=O)O)cc3ccoc23)c1 ; CC(C)Cc1cc2cc(COc3ccccc3CC(=O)O)cc(-c3cccc(CN)c3F)c2o1 ; CC(C)Cc1cc2cc(COc3ccccc3CC(=O)O)cc(-c3ccnc(CN)c3)c2o1 ; CC(C)Cc1cc2cc(COc3ccccc3CC(=O)O)cc(-c3ccnc(CN)c3F)c2o1 ; Nc1ccc(-c2cc(COc3ccccc3CC(=O)O)cc3ccoc23)cc1] 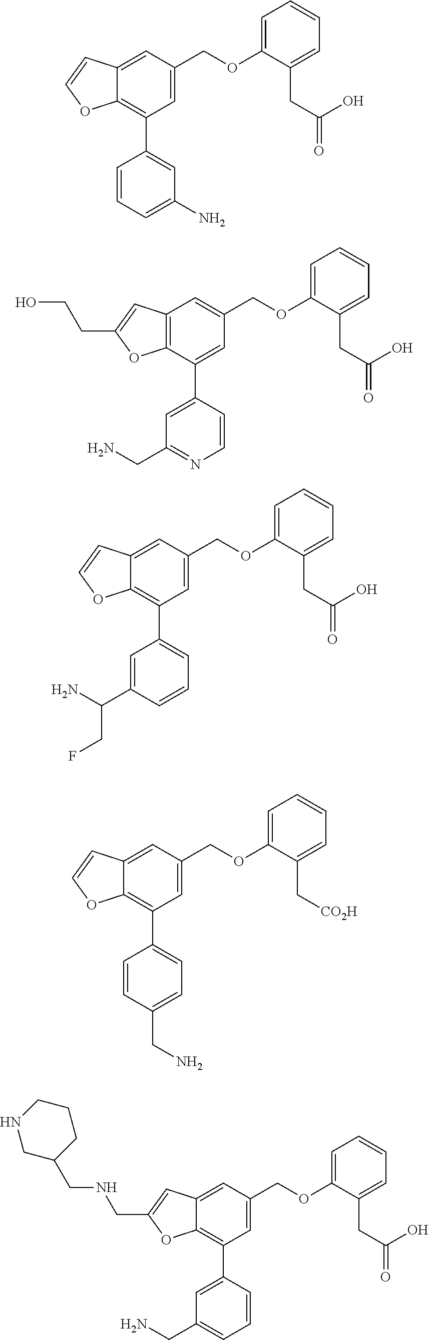 [SMILES: Nc1cccc(-c2cc(COc3ccccc3CC(=O)O)cc3ccoc23)c1 ; NCc1cc(-c2cc(COc3ccccc3CC(=O)O)cc3cc(CCO)oc23)ccn1 ; NC(CF)c1cccc(-c2cc(COc3ccccc3CC(=O)O)cc3ccoc23)c1 ; NCc1ccc(-c2cc(COc3ccccc3CC(=O)O)cc3ccoc23)cc1 ; NCc1cccc(-c2cc(COc3ccccc3CC(=O)O)cc3cc(CNCC4CCCNC4)oc23)c1] 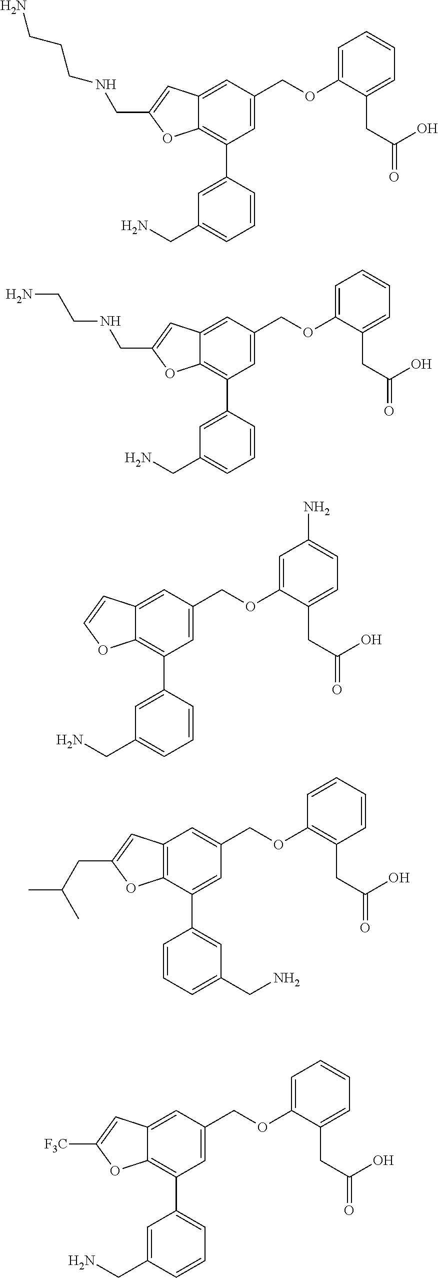 [SMILES: NCCCNCc1cc2cc(COc3ccccc3CC(=O)O)cc(-c3cccc(CN)c3)c2o1 ; NCCNCc1cc2cc(COc3ccccc3CC(=O)O)cc(-c3cccc(CN)c3)c2o1 ; NCc1cccc(-c2cc(COc3cc(N)ccc3CC(=O)O)cc3ccoc23)c1 ; CC(C)Cc1cc2cc(COc3ccccc3CC(=O)O)cc(-c3cccc(CN)c3)c2o1 ; NCc1cccc(-c2cc(COc3ccccc3CC(=O)O)cc3cc(C(F)(F)F)oc23)c1] 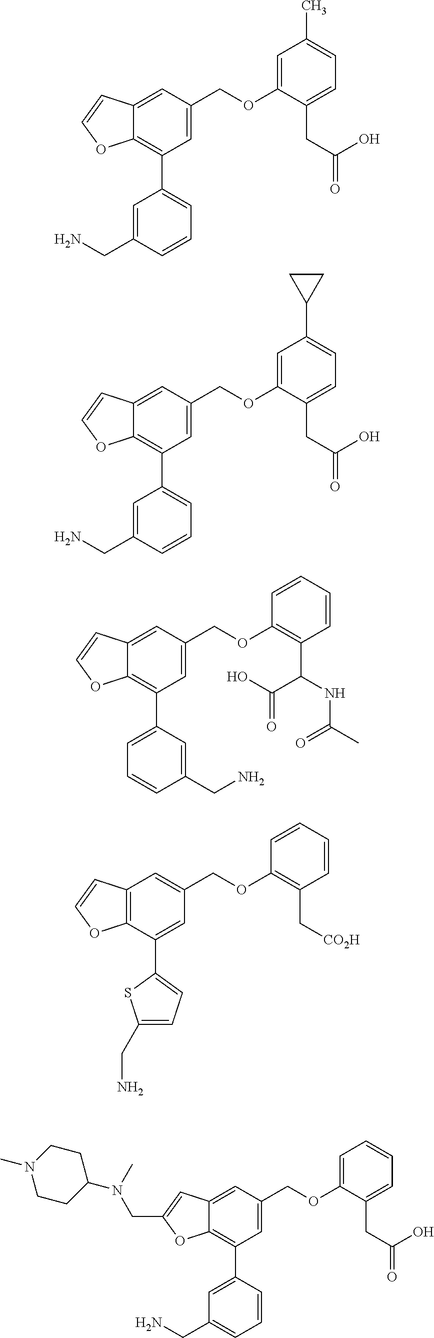 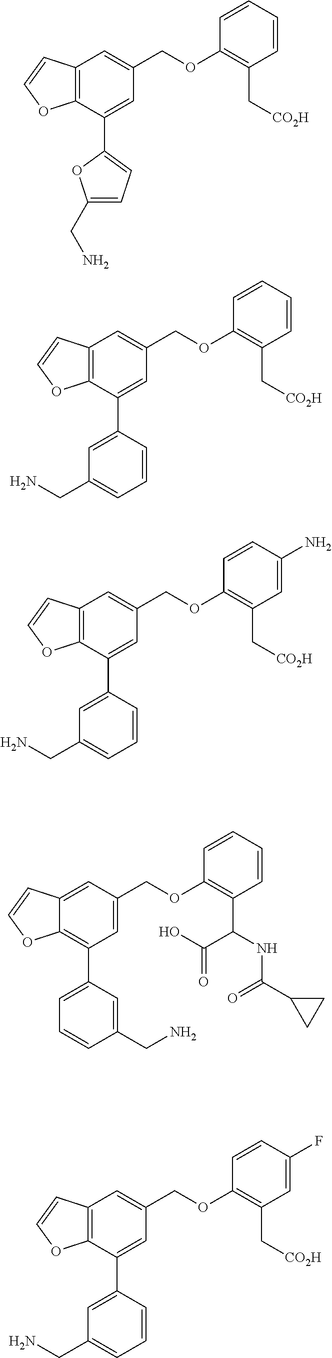 [SMILES: NCc1ccc(-c2cc(COc3ccccc3CC(=O)O)cc3ccoc23)o1 ; NCc1cccc(-c2cc(COc3ccccc3CC(=O)O)cc3ccoc23)c1 ; NCc1cccc(-c2cc(COc3ccc(N)cc3CC(=O)O)cc3ccoc23)c1 ; NCc1cccc(-c2cc(COc3ccccc3C(NC(=O)C3CC3)C(=O)O)cc3ccoc23)c1 ; NCc1cccc(-c2cc(COc3ccc(F)cc3CC(=O)O)cc3ccoc23)c1] 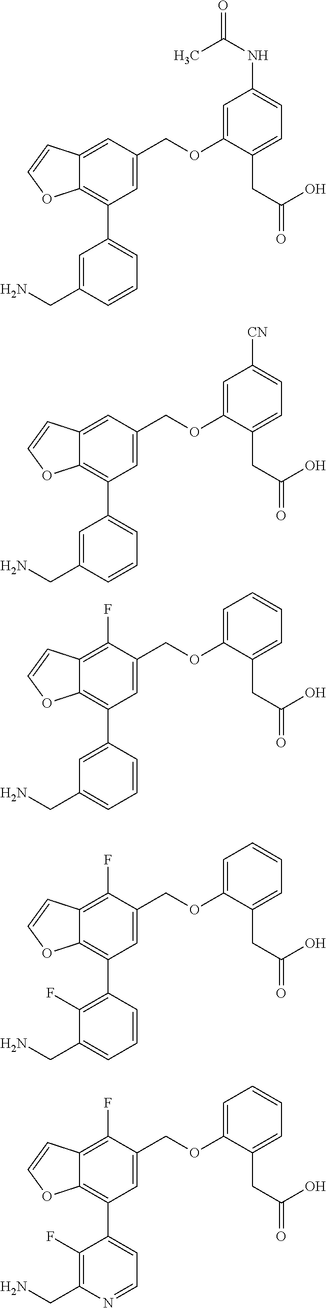 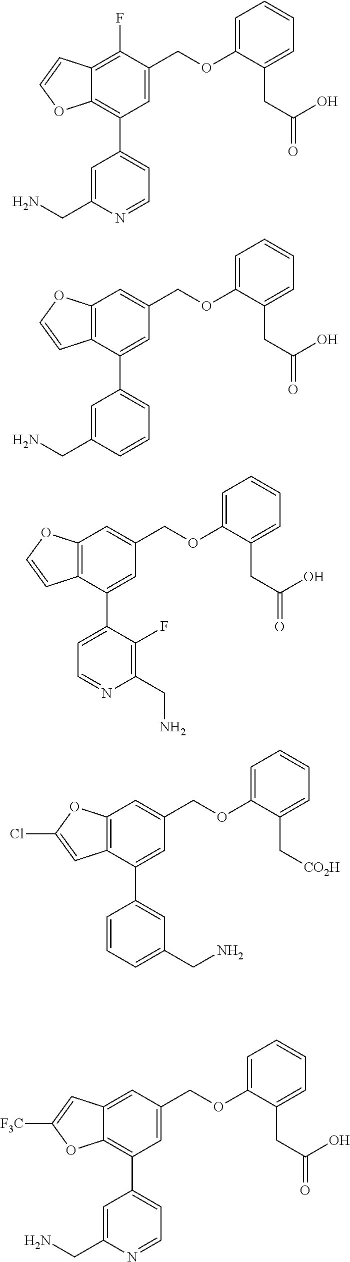 [SMILES: NCc1cc(-c2cc(COc3ccccc3CC(=O)O)c(F)c3ccoc23)ccn1 ; NCc1cccc(-c2cc(COc3ccccc3CC(=O)O)cc3occc23)c1 ; NCc1nccc(-c2cc(COc3ccccc3CC(=O)O)cc3occc23)c1F ; NCc1cccc(-c2cc(COc3ccccc3CC(=O)O)cc3oc(Cl)cc23)c1 ; NCc1cc(-c2cc(COc3ccccc3CC(=O)O)cc3cc(C(F)(F)F)oc23)ccn1] 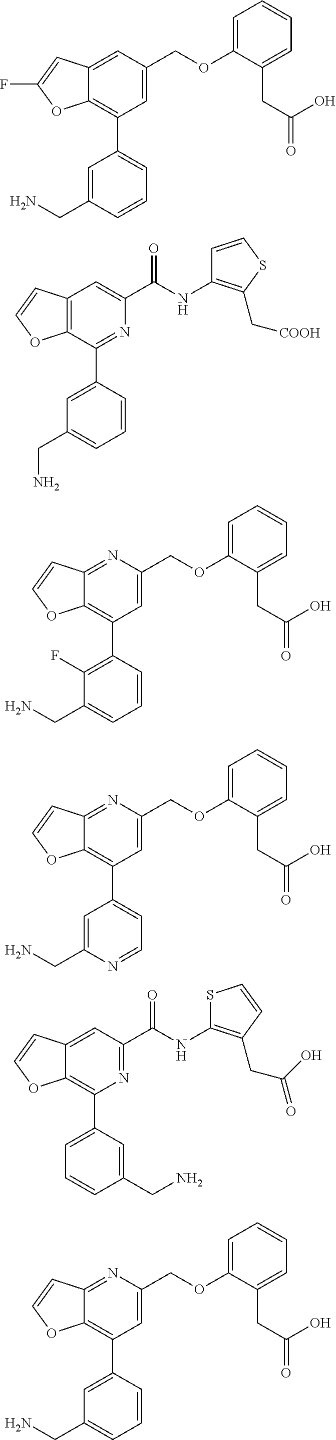 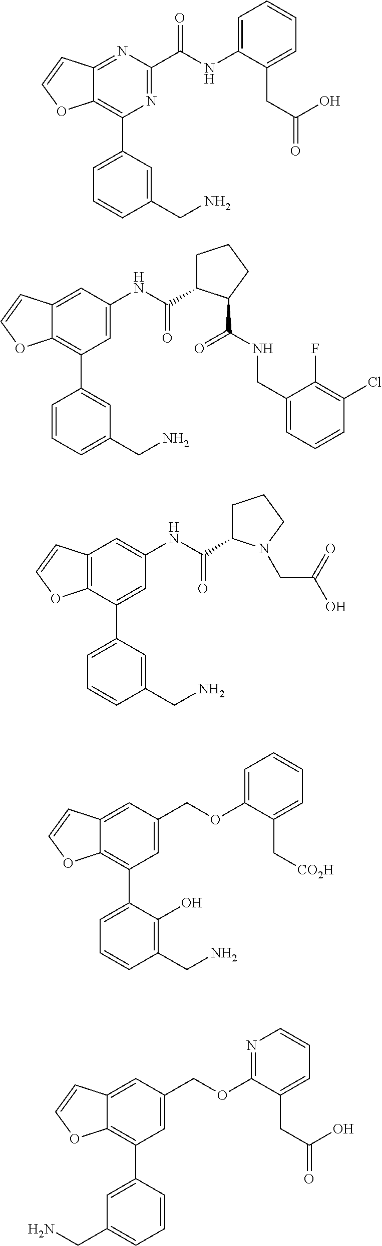 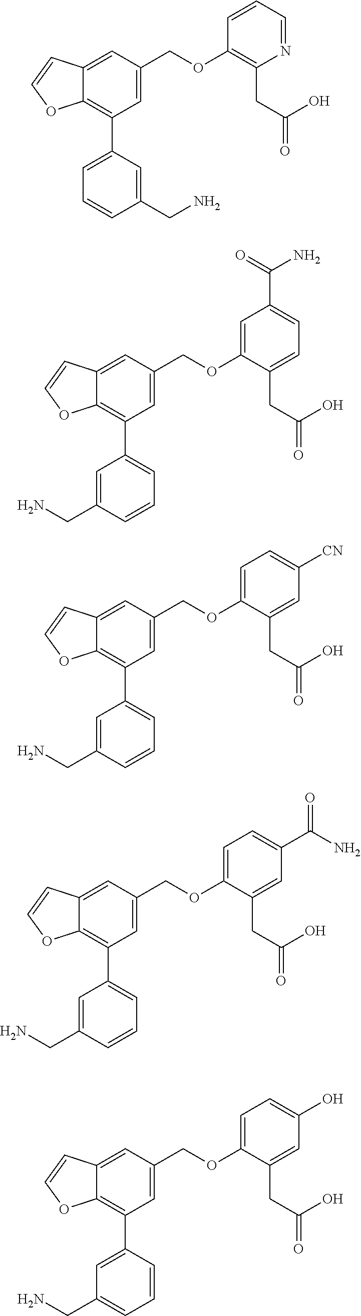 [SMILES: NCc1cccc(-c2cc(COc3cccnc3CC(=O)O)cc3ccoc23)c1 ; NCc1cccc(-c2cc(COc3cc(C(N)=O)ccc3CC(=O)O)cc3ccoc23)c1 ; N#Cc1ccc(OCc2cc(-c3cccc(CN)c3)c3occc3c2)c(CC(=O)O)c1 ; NCc1cccc(-c2cc(COc3ccc(C(N)=O)cc3CC(=O)O)cc3ccoc23)c1 ; NCc1cccc(-c2cc(COc3ccc(O)cc3CC(=O)O)cc3ccoc23)c1] 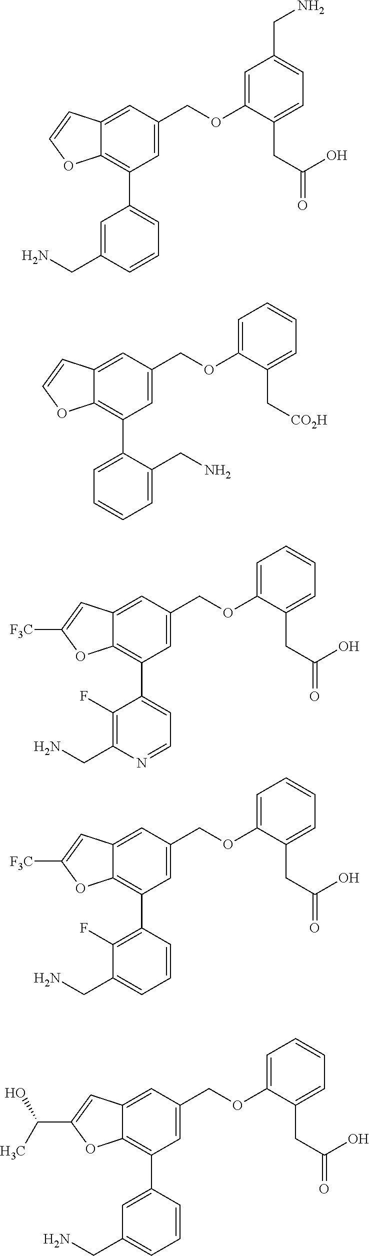 [SMILES: NCc1cccc(-c2cc(COc3cc(CN)ccc3CC(=O)O)cc3ccoc23)c1 ; NCc1ccccc1-c1cc(COc2ccccc2CC(=O)O)cc2ccoc12 ; NCc1nccc(-c2cc(COc3ccccc3CC(=O)O)cc3cc(C(F)(F)F)oc23)c1F ; NCc1cccc(-c2cc(COc3ccccc3CC(=O)O)cc3cc(C(F)(F)F)oc23)c1F ; C[C@H](O)c1cc2cc(COc3ccccc3CC(=O)O)cc(-c3cccc(CN)c3)c2o1] 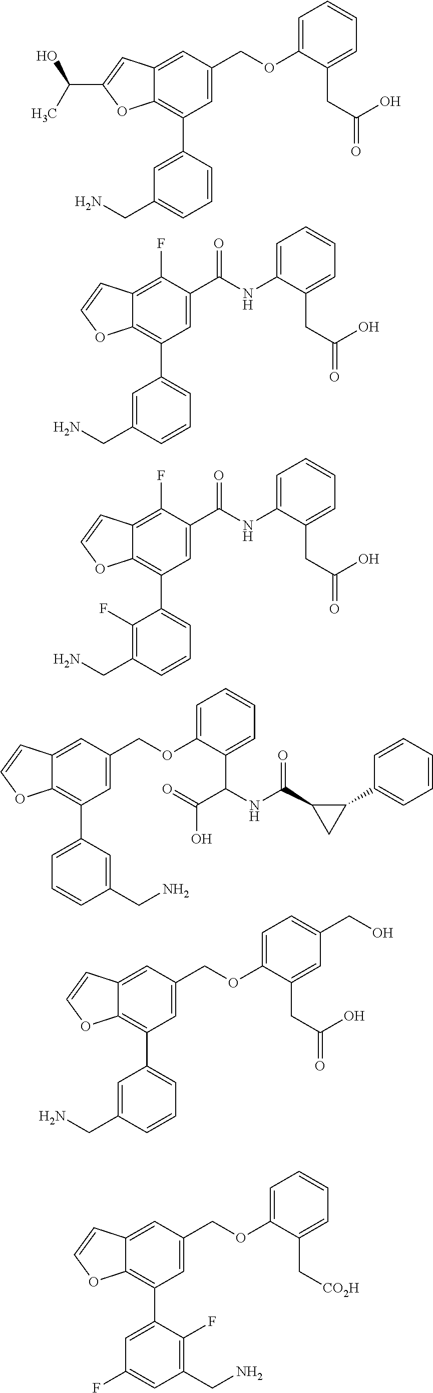 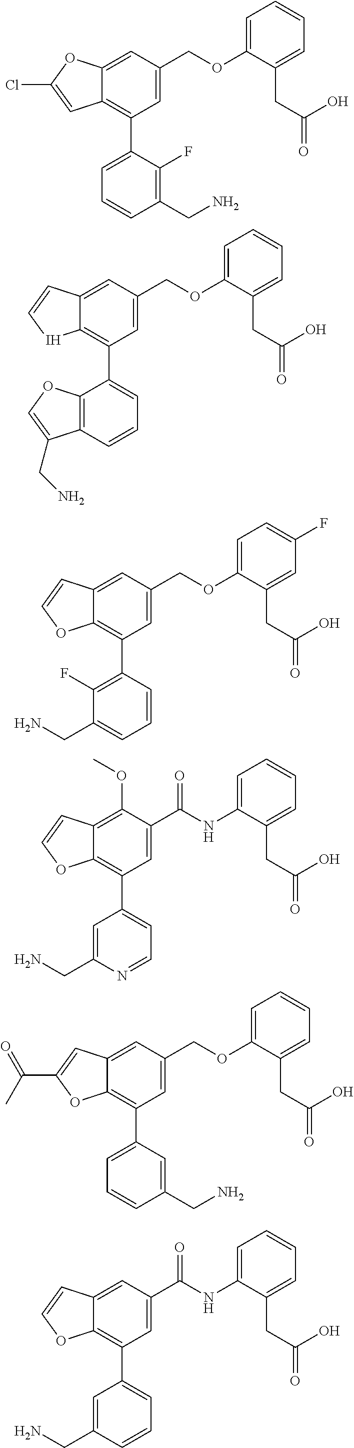 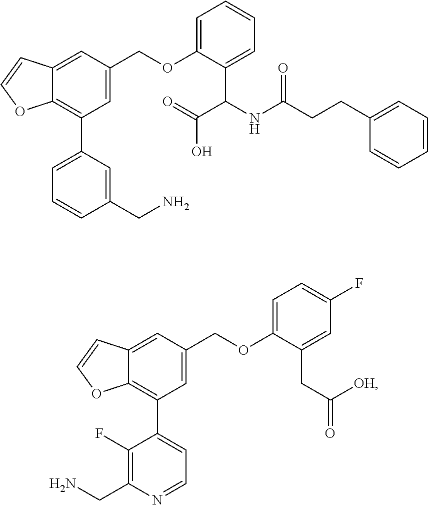 [SMILES: NCc1cccc(-c2cc(COc3ccccc3C(NC(=O)CCc3ccccc3)C(=O)O)cc3ccoc23)c1 ; NCc1nccc(-c2cc(COc3ccc(F)cc3CC(=O)O)cc3ccoc23)c1F]
or a pharmaceutically acceptable salt or prodrug thereof.

25. The method of claim 24, wherein the disease or condition characterized by aberrant complement system activity is an immunological disorder.

26. The method of claim 24, wherein the disease or condition characterized by aberrant complement system activity is a disease of the central nervous system.

27. The method of claim 24, wherein the disease or condition characterized by aberrant complement system activity is a neurodegenerative disease or neurological disease.

28. The method of claim 24, wherein the disease or condition characterized by aberrant complement system activity is a renal disease.

29. The method of claim 24, wherein the disease or condition characterized by aberrant complement system activity is a cardiovascular disease.

30. The method of claim 24, wherein the disease or condition characterized by aberrant complement system activity is selected from the group consisting of antineutrophil cytoplasmic antibody (ANCA)-associated vasculitis (AAV), warm autoimmune hemolytic anemia, IgA nephropathy, C3 glomerulonephritis, and focal segmental glomerulosclerosis.

31. The method of claim 24, wherein the disease or condition characterized by aberrant complement system activity is a hematological disorder.

32. The method of claim 24, wherein the disease or condition characterized by aberrant complement system activity is an ocular disorder or an eye disorder.

33. The method of claim 24, wherein the disease or condition characterized by aberrant complement system activity is macular degeneration, age-related macular degeneration (AMD), macular edema, diabetic macular edema, choroidal neovascularization (CNV), uveitis, Behcet's uveitis, proliferative diabetic retinopathy, non-proliferative diabetic retinopathy, glaucoma, hypertensive retinopathy, a corneal neovascularization disease, post-corneal transplant rejection, a corneal dystrophic disease, an autoimmune dry eye disease, Stevens-Johnson syndrome, Sjogren's syndrome, an environmental dry eye disease, Fuchs' endothelial dystrophy, retinal vein occlusion, or post-operative inflammation.

34. The method of claim 24, wherein the disease or condition characterized by aberrant complement system activity is selected from the group consisting of paroxysmal nocturnal hemoglobinuria (PNH), atypical hemolytic uremic syndrome, organ transplant rejection, myasthenia gravis, neuromyelitis optica, membranoproliferative glomerulonephritis, dense-deposit disease, cold agglutinin disease, and catastrophic antiphospholipid syndrome.

35. The method of claim 34, wherein the disease is PNH.

36. The method of claim 35, wherein the PNH is characterized by one or more of hemolytic anemia, thrombosis, and impaired bone marrow function.

37. The method of any one of claims 34-36, wherein the orally administering suppresses hemolysis of erythrocytes or blocks C3 fragment deposition on erythrocytes or both.

38. The method of claim 37, wherein the suppression is greater than or equal to about 50%.

39. The method of claim 37, wherein the suppression is greater than or equal to about 80%.

40. The method of claim 37, wherein the suppression is greater than or equal to about 90%.

41. The method of claim 37, wherein the suppression is greater than or equal to about 95%.

42. The method of any one of claims 24-41, wherein the therapeutically effective amount is about 1 mg to about 1500 mg of the compound or a pharmaceutically acceptable salt or prodrug thereof.

43. The method of claim 42, wherein the therapeutically effective amount is about 1 mg to about 1200 mg of the compound or a pharmaceutically acceptable salt or prodrug thereof.

44. The method of claim 42, wherein the therapeutically effective amount is about 1 to about 1000 mg of the compound or a pharmaceutically acceptable salt or prodrug thereof.

45. The method of claim 42, wherein the therapeutically effective amount is about 1 to about 800 mg of the compound or a pharmaceutically acceptable salt or prodrug thereof.

46. The method of claim 42, wherein the therapeutically effective amount is about 1 to about 600 mg of the compound or a pharmaceutically acceptable salt or prodrug thereof.

47. The method of claim 42, wherein the therapeutically effective amount is about 1 to about 400 mg of the compound or a pharmaceutically acceptable salt or prodrug thereof.

48. The method of claim 42, wherein the therapeutically effective amount is about 1 to about 300 mg of the compound or a pharmaceutically acceptable salt or prodrug thereof.

49. The method of claim 42, wherein the therapeutically effective amount is about 1 to about 200 mg of the compound or a pharmaceutically acceptable salt or prodrug thereof.

50. The method of claim 42, wherein the therapeutically effective amount is about 1 to about 100 mg of the compound or a pharmaceutically acceptable salt or prodrug thereof.

51. The method of claim 42, wherein the therapeutically effective amount is about 10 mg of the compound or a pharmaceutically acceptable salt or prodrug thereof.

52. The method of claim 42, wherein the therapeutically effective amount is about 30 mg of the compound or a pharmaceutically acceptable salt or prodrug thereof.

53. The method of claim 42, wherein the therapeutically effective amount is about 100 mg of the compound or a pharmaceutically acceptable salt or prodrug thereof.

54. The method of claim 42, wherein the therapeutically effective amount is about 200 mg of the compound or a pharmaceutically acceptable salt or prodrug thereof.

55. The method of claim 42, wherein the therapeutically effective amount is about 300 mg of the compound or a pharmaceutically acceptable salt or prodrug thereof.

56. The method of claim 42, wherein the therapeutically effective amount is about 400 mg of the compound or a pharmaceutically acceptable salt or prodrug thereof.

57. The method of claim 42, wherein the therapeutically effective amount is about 600 mg of the compound or a pharmaceutically acceptable salt or prodrug thereof.

58. The method of claim 42, wherein the therapeutically effective amount is about 800 mg of the compound or a pharmaceutically acceptable salt or prodrug thereof.

59. The method of claim 42, wherein the therapeutically effective amount is about 1000 mg of the compound or a pharmaceutically acceptable salt or prodrug thereof.

60. The method of claim 42, wherein the therapeutically effective amount is about 1200 mg of the compound or a pharmaceutically acceptable salt or prodrug thereof.

61. The method of any one of claims 24-60, wherein the compound or a pharmaceutically acceptable salt or prodrug thereof is administered once daily.

62. The method of any one of claims 24-60, wherein the compound or a pharmaceutically acceptable salt or prodrug thereof is administered twice daily.

63. The method of any one of claims 24-60, wherein the compound or a pharmaceutically acceptable salt or prodrug thereof is administered three times daily.

64. The method of any one of claims 24-63, wherein the compound or a pharmaceutically acceptable salt or prodrug thereof is administered as an oral dosage form of any one of claims 1-23.
